(19) United States
(12) Patent Application Publication
(10) Pub. No.: US 2025/0144231 A1
(43) Pub. Date: May 8, 2025

(54) COMPOSITIONS AND METHODS FOR LOCALIZED DELIVERY OF CYTOKINES FOR ADOPTIVE CELL THERAPY

(71) Applicant: President and Fellows of Harvard College, Cambridge, MA (US)

(72) Inventors: David J. Mooney, Cambridge, MA (US); Yutong Liu, Cambridge, MA (US); Hua Wang, Cambridge, MA (US)

(73) Assignee: President and Fellows of Harvard College, Cambridge, MA (US)

(21) Appl. No.: 18/912,122

(22) Filed: Oct. 10, 2024

Related U.S. Application Data
(63) Continuation of application No. PCT/US2023/018573, filed on Apr. 13, 2023.
(60) Provisional application No. 63/330,562, filed on Apr. 13, 2022.

(51) Int. Cl. A61K 47/69; A61K 38/20; A61P 35/00

(52) U.S. Cl. CPC A61K 47/6901; A61K 38/208; A61P 35/00

(57) ABSTRACT

Disclosed herein are compositions and methods for metabolically labeling cells using click chemistry reagents. The compositions and methods disclosed herein provide a specific and efficient means of localizing desired agents, such as anti-tumor cytokines, to a variety of cell types for adoptive cell therapy.

### RELATED APPLICATIONS

The instant application is a continuation application of International Patent Application No. PCT/US2023/018573, filed on Apr. 13, 2023, which claims priority to U.S. Provisional Application No. 63/330,562, filed on Apr. 13, 2022, the entire contents of which are expressly incorporated herein by reference.

### GOVERNMENT SUPPORT

This invention was made with government support under CA244726 awarded by National Institutes of Health (NIH) and under FD006589 awarded by U.S. Food and Drug Administration (FDA). The government has certain rights in this invention.

### BACKGROUND OF THE INVENTION

Cell-based immunotherapy has emerged as a promising treatment for various human diseases, such as infections, hematological malignancies, autoimmune diseases and cancers. Adoptive cell therapy (ACT), in which ex vivo activated/expanded antigen-specific T cells are administered to patients, is one of the more promising cancer treatments under development. These strategies involve the use of autologous immune cells, e.g., T cells, isolated from patients that are activated/expanded ex vivo and then reinfused to kill cancer cells. However, adoptive cell therapy has limited efficacy in the treatment of most solid tumors due to poor immune cell functionality and persistence after transfer into patients, particularly within the immunosuppressive tumor microenvironment (TME).

Strategies for mitigating the immunosuppressive TME may be required for successful adoptive cell therapy against solid tumors. Systemic cytokine administration is one approach under development for enhancing the antitumor responses of adoptive cell therapy, however, toxic side effects are associated with systemic cytokine administration at the high doses required to achieve therapeutic outcomes.

Additionally, strategies for generating regulatory T (Treg) cells ex vivo as a therapy for autoimmune diseases and transplant rejection are underway. However, Tregs may been found to have functional plasticity and produce pro-inflammatory cytokines at the site of inflammation, which limit their efficacy. Thus, for Treg therapy to be successful, it may be required to develop new ways to maintain and enhance the suppressive function of Tregs in vivo.

### SUMMARY OF THE INVENTION

Disclosed herein are compositions and methods for metabolically labeling and targeting immune cells using click chemistry reagents. The compositions and methods disclosed herein provide a specific and efficient means of localizing desired agents, such as cytokines, to a variety of cell types in vivo, ex vivo, and in vitro.

Adoptive T cell transfer (ACT) therapies can suffer from a number of limitations (e.g., poor control of solid tumors), and while combining ACT with cytokine therapy can enhance effectiveness, this approach can also result in significant side effects resulting from cytokines freely circulating in the body, leading to toxicity and potentially lethal inflammatory syndromes. In addition, cytokines are often cleared from the body too fast to produce the desired cancer therapeutic effects.

The present invention is based, at least in part, on the discovery that a nanotechnology approach can improve the efficacy of ACT therapies by metabolically labeling immune cells, such as T cells, e.g., directly and/or indirectly with unnatural sugar molecules, thereby allowing conjugation of desired agents, such as antitumor cytokines, onto the immune cell surface during the manufacturing process. In certain embodiments, the compositions and methods described herein utilize an engineered sugar molecule that can be taken up by immune cells, such as T cells, and integrated into the complex sugar chains that decorate the cells' surfaces, which can then be used to conjugate desired agents, such as antitumor cytokines. The compositions and methods disclosed herein can allow local, concentrated activity of otherwise toxic cytokines. The concentrated cytokines can locally enhance T cell functions without producing unwanted systemic side effects. The compositions and methods described herein can increase immune cell, e.g., T cell, infiltration into solid tumors, activate the host immune system toward a Type 1 response, encourage antigen spreading, improve control of aggressive solid tumors, and/or achieve complete cancer regression at doses of ACT therapies, such as CAR-T cells, that were too low to cure the cancer (non-curative doses) using unmodified immune cells. The compositions and methods described herein can provide an effective and easily integrated approach to the current ACT manufacturing process to increase efficacy in various settings.

In certain embodiments, the compositions and methods described herein can be used to metabolically label immune cells, such as T cells, directly and/or indirectly with unnatural sugar molecules, such as reactive azido sugar. In some embodiments, the reactive azido sugar can be delivered to immune cells, such as T cells, via nanoparticles. In certain embodiments, the delivery of the unnatural sugar molecule, e.g., reactive azido sugar, can be in vivo and/or in situ. In certain embodiments, the delivery of the unnatural sugar molecule, e.g., reactive azido sugar, can be in vitro and/or ex vivo (e.g., in a culture dish). In some embodiments, the reactive azido sugar can be delivered to immune cells, such as T cells, without using nanoparticles.

In certain embodiments, the compositions and methods described herein can be used to metabolically label immune cells, such as T cells, using click-chemistry. In certain embodiments, the compositions and methods described herein can exploit the sugar molecules' azido group to link to specific agents, such as cytokine molecules, that are modified with a highly compatible chemical group, such as dibenzocyclooctyne (DBCO).

Unnatural sugars and unnatural sugar nanoparticles, such as azido-sugars and azido-sugar nanoparticles, can be used to metabolically label cell-surface glycoproteins on immune cells with azido groups. The azido-label can enable targeted delivery of agents, such as dibenzocyclooctyne (DBCO)-coupled agents, to immune cells via click chemistry. Accordingly, in one aspect, the present invention is related to the metabolic glycoengineering of unnatural sugars and unnatural sugar nanoparticles, including azido-sugars and azido-sugar nanoparticles, to label immune cell membranes with click chemistry reagents, e.g., azido groups, for subsequent targeted delivery of agents coupled to another click reagent, including dibenzocyclooctyne (DBCO)-coupled agents, via click chemistry. In some embodiments, the present invention provides immune cells labelled with a click chemistry reagent (e.g., an azido group) suitable for use in an adoptive cell therapy. In some embodiments, the present invention provides immune cells conjugated to a cytokine, for example, a cytokine comprising a dibenzocyclooctyne (DBCO) group, suitable for use in an adoptive cell therapy. The present invention is predicated on the surprising finding that a method of using cytokines comprising a low number (e.g., 1-10) dibenzocyclooctyne (DBCO) groups can serve to both (1) maintain the structure and function of the cytokine, and (2) achieve binding of the cytokine to the immune cell. The success and efficacy using cytokines comprising a low number (e.g., 1-10) of dibenzocyclooctyne (DBCO) was not reasonably expected, particularly given the challenges associated with providing cytokine coupled to click reagents in an amount sufficient to target and covalently label a cell-surface glycoprotein comprising another click reagent, e.g., through a selective reaction between an incorporated azide group and a DBCO, without altering the cytokine bioactivity, e.g., via denaturation. The unpredictability and challenge in achieving such efficacy stems at least in part from the tendency of click reagents to induce conformation changes in the cytokine.

In particular, this disclosure provides methods and compositions for improving adoptive cell therapy, such as chimeric antigen receptor (CAR) T cell therapy, via the targeted delivery of cytokines to solid tumors to reduce or eliminate the toxic side effects associated with systemic cytokine administration. Without wishing to be bound by any particular theory, most anti-tumor cytokines, when presented to T cells in large amounts in the native soluble form, exhibit high toxicity and prevent T cells from proliferating and surviving. However, as described herein, when cytokines are conjugated to T cell surfaces they surprisingly exhibit less toxicity and inhibition on T cell proliferation.

Accordingly, in one aspect, this disclosure provides methods and compositions for adoptive cell therapy comprising conjugating a cytokine onto an immune cell, e.g., T cell, surface. In some embodiments, an immune cell, e.g., T cell, conjugated with a cytokine may have similar or increased viability and/or proliferation compared with an unconjugated immune cell, e.g., T cell. In some embodiments, an immune cell, e.g., T cell, conjugated with a cytokine may have similar or increased viability and/or proliferation compared with an unconjugated immune cell, e.g., T cell, contacted with soluble cytokine. In some embodiments, T cells conjugated with cytokines may direct T cell differentiation in substantially the same manner as soluble cytokines, for example, as demonstrated by CD4/CD8 ratio, memory, activation, and/or exhaustion markers.

In one aspect, the present invention provides a method of preventing or treating a cancer in a subject, comprising administering to the subject an adoptive cell therapy, wherein the adoptive cell therapy comprises: (i) an immune cell comprising a cell-surface glycoprotein coupled to a first click reagent; and (ii) a cytokine coupled to a second click reagent, wherein the cell-surface glycoprotein is covalently linked to the cytokine through a selective reaction between the first click reagent and the second click reagent, thereby preventing or treating the cancer.

In one aspect, the present invention provides a method of preventing or treating an autoimmune disease in a subject, comprising administering to the subject an adoptive cell therapy, wherein the adoptive cell therapy comprises: (i) an immune cell comprising a cell-surface glycoprotein coupled to a first click reagent; and (ii) a cytokine coupled to a second click reagent, wherein the cell-surface glycoprotein is covalently linked to the cytokine through a selective reaction between the first click reagent and the second click reagent, thereby preventing or treating the autoimmune disease.

In one aspect, the present invention provides a method of preventing or treating a viral disease in a subject, comprising administering to the subject an adoptive cell therapy, wherein the adoptive cell therapy comprises: (i) an immune cell comprising a cell-surface glycoprotein coupled to a first click reagent; and (ii) a cytokine coupled to a second click reagent, wherein the cell-surface glycoprotein is covalently linked to the cytokine through a selective reaction between the first click reagent and the second click reagent, thereby preventing or treating the viral disease.

In one aspect, the present invention provides a method of enhancing an immune response against a cancer in a subject, comprising administering to the subject an adoptive cell therapy, wherein the adoptive cell therapy comprises: (i) an immune cell comprising a cell-surface glycoprotein coupled to a first click reagent; and (ii) a cytokine coupled to a second click reagent, wherein the cell-surface glycoprotein is covalently linked to the cytokine through a selective reaction between the first click reagent and the second click reagent, thereby enhancing the immune response against the cancer.

In one aspect, the present invention provides a method of enhancing an immune response against a virus in a subject, comprising administering to the subject an adoptive cell therapy, wherein the adoptive cell therapy comprises: (i) an immune cell comprising a cell-surface glycoprotein coupled to a first click reagent; and (ii) a cytokine coupled to a second click reagent, wherein the cell-surface glycoprotein is covalently linked to the cytokine through a selective reaction between the first click reagent and the second click reagent, thereby enhancing the immune response against the virus.

In one aspect, the present invention provides a method of reducing or preventing an immune response against a self-antigen in a subject, comprising administering to the subject an adoptive cell therapy, wherein the adoptive cell therapy comprises: (i) an immune cell comprising a cell-surface glycoprotein coupled to a first click reagent; and (ii) a cytokine coupled to a second click reagent, wherein the cell-surface glycoprotein is covalently linked to the cytokine through a selective reaction between the first click reagent and the second click reagent, thereby reducing or preventing an immune response against a self-antigen.

In one aspect, the present invention provides a method of delivering a non-toxic level of cytokine to a subject in need thereof, comprising administering to the subject an adoptive cell therapy, wherein the adoptive cell therapy comprises: (i) an immune cell comprising a cell-surface glycoprotein coupled to a first click reagent; and (ii) a cytokine coupled to a second click reagent, wherein the cell-surface glycoprotein is covalently linked to the cytokine through a selective reaction between the first click reagent and the second click reagent, thereby delivering a non-toxic level of cytokine to a subject in need thereof.

In various embodiments of the above aspects or any other aspect of the invention delineated herein, the method may comprise contacting the immune cell with an unnatural sugar and/or an unnatural sugar nanoparticle to produce the immune cell comprising the cell-surface glycoprotein coupled to a first click reagent.

In one aspect, the present invention provides a method of treating a subject in need thereof with an adoptive cell therapy, comprising: (i) contacting an immune cell with an unnatural sugar and/or an unnatural sugar nanoparticle to produce an immune cell comprising a cell-surface glycoprotein coupled to a first click reagent; and (ii) administering to the subject the immune cell comprising the cell-surface glycoprotein coupled to the first click reagent, and a cytokine coupled to a second click reagent, wherein the cell-surface glycoprotein is covalently linked to the cytokine through a selective reaction between the first click reagent and the second click reagent, thereby treating a subject in need thereof with an adoptive cell therapy.

In various embodiments of the above aspects or any other aspect of the invention delineated herein, the unnatural sugar and/or an unnatural sugar nanoparticle comprises the first click reagent. In some embodiments, the first click reagent is selected from the group consisting of an azide group, a dibenzocyclooctyne (DBCO) group, a transcyclooctene group, a tetrazine group, a norbornene group, and variants thereof. In some embodiments, the first click reagent comprises an azide group. In some embodiments, the unnatural sugar and/or an unnatural sugar nanoparticle is an unnatural azido-sugar and/or an unnatural azido-sugar nanoparticle.

In various embodiments of the above aspects or any other aspect of the invention delineated herein, the second click reagent is selected from the group consisting of an azide group, a dibenzocyclooctyne (DBCO) group, a transcyclooctene group, a tetrazine group, a norbornene group, and variants thereof. In some embodiments, the second click reagent comprises a dibenzocyclooctyne (DBCO) group.

In various embodiments of the above aspects or any other aspect of the invention delineated herein, the first click reagent comprises an azide group and the second click reagent comprises a dibenzocyclooctyne (DBCO) group. In some embodiments, the DBCO group is coupled to a primary amine of the cytokine. In some embodiments, the cytokine is coupled to between 1-10 (e.g., 1, 2, 3, 4, 5, 6, 7, 8, 9, or 10) DBCO groups. In some embodiments, the cytokine is coupled to 1, 2, or 3 DBCO groups. In some embodiments, the cytokine is coupled to at least 1 DBCO group. In some embodiments, the cytokine is coupled to no more than 1 DBCO group. In some embodiments, the cytokine is coupled to at least 2 DBCO groups. In some embodiments, the cytokine is coupled to no more than 2 DBCO groups. In some embodiments, the cytokine is coupled to at least 3 DBCO groups. In some embodiments, the cytokine is coupled to no more than 3 DBCO groups.

In various embodiments of the above aspects or any other aspect of the invention delineated herein, the cytokine is an anti-tumor cytokine. In some embodiments, (i) the cytokine is selected from the group consisting of interleukins, interferons, chemokines, tumor necrosis factors, and colony stimulating factors of immune cell precursors; (ii) the cytokine is an interleukin selected from the group consisting of IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, and IL-35; (iii) the cytokine is a chemokine selected from the group consisting of CCL family, CXCL family, CX3CL family, and XCL family chemokines; (iv) the cytokine is an inflammatory cytokine selected from the group consisting of IFN-γ, IL-1, and TNF-α; (v) the cytokine is selected from the group consisting of interleukin-2 (IL-2), interleukin-7 (IL-7), interleukin-10 (IL-10), interleukin-12 (IL-12), interleukin-15 (IL-15), IL-15/IL-15Ra, interleukin-18 (IL-18), interleukin-21 (IL-21), interleukin-27 (IL-27), interferon-α (IFN-α), interferon- [symbol] (IFN- [symbol] , granulocyte macrophage-colony stimulating factor (GM-CSF), Fms-like tyrosine kinase receptor 3 ligand (Flt3-L), tumour necrosis factor α (TNF-α), and combinations thereof; and/or (vi) the cytokine is an anti-tumor cytokine. In some embodiments, the cytokine is selected from the group consisting of interleukin-2 (IL-2), interleukin-7 (IL-7), interleukin-10 (IL-10), interleukin-12 (IL-12), interleukin-15 (IL-15), IL-15/IL-15Rα, interleukin-18 (IL-18), interleukin-21 (IL-21), interleukin-27 (IL-27), interferon-α (IFN-α), interferon- [symbol] (IFN- [symbol] ), granulocyte macrophage-colony stimulating factor (GM-CSF), Fms-like tyrosine kinase receptor 3 ligand (Flt3-L), tumour necrosis factor α (TNF-α), and combinations thereof. In some embodiments, the cytokine comprises interleukin-2 (IL-2). In some embodiments, the cytokine comprises IL-15/IL-15Rα.

In various embodiments of the above aspects or any other aspect of the invention delineated herein, cytokine is administered to the subject prior to, concurrently with, or after the administration of the immune cell.

In various embodiments of the above aspects or any other aspect of the invention delineated herein, the selective reaction between the first click reagent and the second click reagent occurs in vitro, ex vivo, or in vivo.

In various embodiments of the above aspects or any other aspect of the invention delineated herein, the immune cell comprises a lymphocyte, optionally a tumor-infiltrating lymphocyte. In some embodiments, the immune cell comprises a T-cell, a B-cell, a natural killer (NK) cell, or a combination thereof. In some embodiments, the immune cell comprises an engineered T cell receptor (TCR). In some embodiments, the immune cell comprises a chimeric antigen receptor (CAR). In various embodiments of the above aspects or any other aspect of the invention delineated herein, the immune cell comprises a regulatory T (Treg) cell.

In various embodiments of the above aspects or any other aspect of the invention delineated herein, the adoptive cell therapy is selected from the group consisting of (i) a tumor-infiltrating lymphocyte (TIL) therapy; (ii) a engineered T cell receptor (TCR) therapy; (iii) a chimeric antigen receptor (CAR) T cell therapy; (iv) a natural killer (NK) cell therapy; and (v) a regulatory T (Treg) cell therapy.

In various embodiments of the above aspects or any other aspect of the invention delineated herein, the immune cell retains its proliferation, viability, memory, activation, and/or exhaustion phenotypes, optionally as determined by CD4/CD8 ratio, memory, activation, and/or exhaustion markers.

In various embodiments of the above aspects or any other aspect of the invention delineated herein, the method reduces tumor size, delays tumor growth, reduces cancer burden, increases survival time, prevents cancer from developing, depletes cancer cells, prevents or reduces cancer relapse, or prevents or reduces cancer recurrence or metastasis, increases T cells infiltration in solid tumors, increases antigen presentation, and/or increases antigen spreading in the subject.

In various embodiments of the above aspects or any other aspect of the invention delineated herein, the method results in the targeted delivery of non-toxic levels of one or more cytokines to the subject and/or the reduction of cytokine-related toxicity and inhibition on immune cell proliferation, e.g., as compared with systemic cytokine administration.

In various embodiments of the above aspects or any other aspect of the invention delineated herein, the method reduces cytokine-related toxicity and inhibition on immune cell proliferation as compared with systemic cytokine administration.

In various embodiments of the above aspects or any other aspect of the invention delineated herein, the subject is suffering from a cancer, a viral disease, and/or an autoimmune disease. In some embodiments, the cancer is selected from the group consisting of a cancer of the digestive system; a hepatic carcinoma; a liver cancer; a colon cancer; an esophageal cancer; a gastric cancer; a hepatoma; a kidney or renal cancer; an oral cavity cancer; a pancreatic cancer; a prostate cancer; a rectal cancer; a stomach cancer; a basal cell carcinoma; a biliary tract cancer; a lung cancer; a bladder cancer; a cervical cancer; an endometrial cancer; a uterine cancer; a blond cancer; a bone cancer; a skin cancer; a cancer of the urinary system; and combinations thereof. In some embodiments, the cancer is selected from the group consisting of a solid tumor, a leukemia, a lymphoma, and a multiple myeloma. In some embodiments, the cancer comprises a solid tumor.

In various embodiments of the above aspects or any other aspect of the invention delineated herein, the immune cell is administered to the subject in the absence of a scaffold.

In various embodiments of the above aspects or any other aspect of the invention delineated herein, the immune cell is administered to the subject prior to, concurrently with, or after the administration of a scaffold, optionally wherein the scaffold comprises an additional agent selected from the group consisting of a growth factor, a differentiation factor, a homing factor, a cytokine, a chemokine, and combinations thereof. In some embodiments, the selective reaction between the first click reagent and the second click reagent occurs in vitro, ex vivo, or in vivo within a scaffold.

In one aspect, the present invention provides a method of producing an adoptive cell therapy, comprising: (i) providing an immune cell comprising a cell-surface glycoprotein; (ii) contacting the immune cell with an unnatural azido-sugar and/or an unnatural azido-sugar nanoparticle to metabolically label the cell-surface glycoprotein with a first click reagent; and (iii) contacting the immune cell with a cytokine coupled to a second click reagent, wherein the cell-surface glycoprotein is covalently linked to the cytokine through a selective reaction between the first click reagent and the second click reagent, thereby producing the adoptive cell therapy. In some embodiments, the unnatural azido-sugar nanoparticle comprises a polymer of azido sugar. In some embodiments, the polymer of azido sugar comprises an tetraacetyl-N-azidoacetylmannosamine (Ac₄ManAz) or a derivative thereof. In some embodiments, the Ac₄ManAz is functionalized with at least one acrylate bond. In some embodiments, the polymer of azido sugar is produced by reversible addition-fragmentation chain-transfer (RAFT) polymerization of Ac₄ManAz to yield poly(azido-sugar)ₙ, wherein n is any integer between 1 and 500 (n=1 (G1) or n=500 (G500)). In some embodiments, the unnatural azido-sugar nanoparticle comprises a G400 nanoparticle.

In one aspect, the present invention provides an immune cell comprising: (i) a cell-surface glycoprotein coupled to a first click reagent; and (ii) a cytokine coupled to a second click reagent; wherein the cell-surface glycoprotein is covalently linked to the cytokine through a selective reaction between the first click reagent and the second click reagent. In some embodiments, the immune cell comprises a lymphocyte, optionally a tumor-infiltrating lymphocyte. In some embodiments, the immune cell comprises a T-cell, a B-cell, a natural killer (NK) cell, or a combination thereof. In some embodiments, the immune cell comprises an engineered T cell receptor (TCR). In some embodiments, the immune cell comprises a chimeric antigen receptor (CAR). In various embodiments of the above aspects or any other aspect of the invention delineated herein, the immune cell comprises a regulatory T (Treg) cell.

In one aspect, the present invention provides composition comprising the immune cells described herein.

In another aspect, the present invention provides a method for direct metabolic labelling and/or targeted modulation of immune cells. Direct metabolic labelling and/or targeted modulation of immune cells in vivo allows for the local delivery of agents, such as cytokines, to improve anti-tumour efficacy. In some embodiments, the present invention provides immune cells metabolically labelled with chemical tags in vitro, ex vivo, and in vivo, allowing for their subsequent targeted modulation over time in a subject.

In another aspect, in one aspect, the present invention provides a method of inducing differentiation and/or proliferation of an immune cell, comprising: contacting an immune cell with an unnatural azido-sugar and/or an unnatural azido-sugar nanoparticle to metabolically label a cell-surface glycoprotein with an azido group; and conjugating a cytokine comprising a dibenzocyclooctyne (DBCO) group to the azido-labelled immune cell via click chemistry, thereby inducing differentiation and/or proliferation of the immune cell.

In another aspect, the present invention provides a method of enhancing the efficacy of an adoptive cell therapy, comprising: contacting an immune cell with an unnatural azido-sugar and/or an unnatural azido-sugar nanoparticle to metabolically label a cell-surface glycoprotein with an azido group; and conjugating a cytokine comprising a dibenzocyclooctyne (DBCO) group to the azido-labelled immune cell via click chemistry, thereby enhancing the efficacy of an adoptive cell therapy.

### BRIEF DESCRIPTION OF THE DRAWINGS

FIGS. 1A-1D shows azido-sugar nanoparticles metabolically label T cells with cell-surface azide groups. FIG. 1A shows a schematic of metabolic labeling of T cells with azido-sugar G400 nanoparticles (NPs). Azido-sugar nanoparticles enter T cells via endocytosis, undergo enzymatic hydrolysis, get integrated into membrane glycoproteins, and present the azide group onto T cell surface. FIG. 1B shows percentage of cell surface azide+ T cells after 3 days of incubation with G400 azido-sugar NPs. FIG. 1C shows median fluorescence intensity (MFI) of T cell surface azide signals after T cells are treated with G400 NPs at various concentrations for 3 days (n=3, one-way ANOVA and Tukey's test). FIG. 1D shows representative fluorescent imaging and quantification of azide signaling from T cells over time. T cells were treated with 200 μM G400 NP until the end of Day 3, after which excessive G400 NP were removed and T cells were cultured free of G400 NP.

FIGS. 2A-2F show azido labeling of T cells allows conjugation of DBCO-cytokines and generate potent cytokine-dependent inflammatory phenotypes. FIG. 2A shows a schematic illustration of conjugating DBCO-cytokines onto azido-labeled T cells. FIG. 2B shows viability data for T cells receiving no IL-12, conjugated with DBCO IL-12, and presented with soluble IL-12 in media in in vitro culture. FIG. 2C shows representative phenotyping data and FIG. 2D shows heatmap data showing memory phenotype markers and activation and exhaustion markers, for T cells conjugated with DBCO-IL-12, treated with soluble IL-12, or temporarily exposed to IL-12 for click reaction time at different G400 NP concentrations. FIG. 2E shows cytolytic activities of Pmel-1 T cells conjugated with DBCO-IL-12, treated with soluble IL-12, or temporarily exposed to IL-12 against B16-F10 tumor cells. FIG. 2F shows ELISA quantification of the amount of DBCO-IL-12 conjugated onto 1 million T cells at various concentrations.

FIGS. 3A-3F show conjugating IL-12 on T cell surfaces increases the efficacy of adoptive T cell transfer therapies and delays tumor growth. FIG. 3A shows schematics of animal study timeline; B16-F10 tumors were inoculated on Day 0, followed by tail vein injection of T cells on Day 5; blood was collected every 6-8 days for flow cytometry analysis after T cell injection. FIG. 3B shows average tumor volume, and FIG. 3C shows mouse survival data over therapeutic study (n=7). FIG. 3D shows number of total T cells per 1 mL of blood and FIG. 3E shows number of CD8+Thy1.1+Pmel-1 T cells per 1 mL of blood over therapeutic study (n=7). FIG. 3F shows percentage and count of different memory population of T cells in blood on Day 17 (n=7).

FIGS. 4A-4I shows conjugating IL-12 on T cell surfaces increases T cell infiltration in solid tumors, increases antigen presentation, and promotes antigen spreading. FIG. 4A shows number of CD8+Thy1.1+ T cells and FIG. 4B shows total T cells per 1000 mm³ tumor (n=5). FIG. 4C shows number of CD4 and FIG. 4D shows CD8 T cells per 1000 mm³ tumor (n=5). FIG. 4E shows percentage of CD8+Thy1.1+ T cells among all T cell in lymph nodes (n=5). FIG. 4F shows MFI of MHC-II expression on dendritic cells (n=5). FIG. 4G shows heatmap of average expression level of Th1 cytokines in CD8+ T cells in lymph nodes and spleen (n=5). FIG. 4H shows representative flow cytometry plots and FIG. 4I shows summary flow cytometry data for OVA-specific CD8+ T cells on Day 15.

FIGS. 5A-5B shows G400 NP could be used to conjugate multiple cytokines and improve the efficacy of CAR-T therapy. FIG. 5A shows mouse survival data of conjugating two cytokines, IL-15/IL-15Ra and IL-12, onto T cells (n=7). FIG. 5B shows number of CD8+Thy1.1+Pmel-1 T cells per 1 mL of blood over therapeutic study (n=7).

FIGS. 6A-6F shows azido-sugar nanoparticles label T cells without affecting T cell function and phenotype. FIG. 6A shows MFI of CFSE, FIG. 6B shows viability, and FIG. 6C shows CD4/CD8 ratios in T cells incubated in various concentrations of G400 NP over time. FIG. 6D shows percentages of T cell populations with different memory phenotypes.

FIG. 6E shows MFI of and FIG. 6F shows percentage of positive cells for various activation and exhaustion markers T cells incubated in various concentrations of G400 NP over time.

FIGS. 7A-7C shows MALDI-TOF spectrum for unmodified and DBCO-modified IL-12 (FIG. 7A), IL-21(FIG. 7B), and TNF-α (FIG. 7C). Each cytokine has on average 2-3 DBCO conjugated.

FIGS. 8A-8C shows comparison of cytokine bioreactivity after DBCO modification with unmodified cytokines for IL-12 (FIG. 8A), IL-21(FIG. 8B), TNF-α (FIG. 8C) via CD4/CD8 ratio, activation, and exhaustion profiling with flow cytometry.

FIGS. 9A-9C shows percentage of IL-12+(FIG. 9A), IL-21+(FIG. 9B), and TNF-α+(FIG. 9C) cells under various concentration of cytokine conjugation.

FIGS. 10A-10C shows conjugating cytokines onto T cells affect T cell phenotype without inducing cytotoxicity. FIG. 10A shows conjugating IL-21 and TNF-α onto T cells does not induce cytotoxicity as unmodified cytokines. FIG. 10B shows representative phenotyping data and FIG. 10C shows heatmap data showing memory phenotype markers and activation and exhaustion markers, for T cells conjugated with DBCO-IL-21 and DBCO-TNF-α, treated with soluble IL-21 and TNF-α, or temporarily exposed to IL-21, and TNF-α. FIG. 10D shows cytolytic activities of Pmel-1 T cells conjugated with DBCO-IL-21 or DBCO-TNF-α, treated with soluble IL-21 or TNF-α, or temporarily exposed to IL-21 or TNF-α against B16-F10 tumor cells.

FIGS. 11A-11C shows conjugating IL-12 on T cell surfaces increases T cell infiltration in solid tumors, increases antigen presentation, and promotes antigen spreading.

FIG. 11A shows percent of DC cells in tumor infiltrated leukocytes. FIG. 11B shows total T cell count in tumors. FIG. 11C shows total number of leukocytes in spleen. FIG. 11D shows total number of Thy1.1+Pmel-1 T cells, and percent of CD4+ and CD8+Thy1.1+ T cells in spleen. FIG. 11E shows total number of Thy1.1+Pmel-1 T cells, and percent of CD4+ and CD8+Thy1.1+ T cells in tumor draining lymph nodes. FIG. 11F shows heatmap of average expression level of Th1 cytokines in CD4+ T cells in lymph nodes and spleen (n=5).

FIGS. 12A-12C shows conjugating multiple DBCO-cytokines achieves synergistic effect on T cells. FIG. 12A shows conjugating IL-15/IL-15Ra onto T cells, and FIG. 12B shows conjugating both IL-15/IL-15Ra and 11-12 onto T cells promote T cell expansion similar or better than supplementing with IL-2. FIG. 12C shows representative phenotyping data showing activation and exhaustion markers, for T cells conjugated with both DBCO-IL-12 and DBCO-IL-15/IL-15Rα, treated with soluble IL-12 and IL-15/IL-15Rα, or temporarily exposed to IL-12 and IL-15/IL-15Ra for click reaction time at different G400 NP concentrations. For FIGS. 12A-12C, the line labeled “continuous IL-15/IL-15Rα” or “continuous IL-15/IL-15Rα+IL-12” is for soluble cytokines; the line labeled “free IL-15/IL-15Ra” or “free IL-15/IL-15Rα+IL-12” is temporarily exposed cytokines; and the line labeled “conjugated IL-15/IL-15Rα” or “conjugated IL-15/IL-15Rα+IL-12” is for the conjugated cytokines.

### DETAILED DESCRIPTION OF THE INVENTION

Disclosed herein are compositions and methods for metabolically labeling cells using click chemistry reagents. The compositions and methods disclosed herein provide a specific and efficient means of delivering desired agents, such as anti-tumor cytokines, to a variety of cell types in vitro, ex vivo, and in vivo.

### I. Definitions

In order that the present invention may be more readily understood, certain terms are first defined.

Unless otherwise defined herein, scientific and technical terms used in connection with the present invention shall have the meanings that are commonly understood by those of ordinary skill in the art. The meaning and scope of the terms should be clear, however, in the event of any latent ambiguity, definitions provided herein take precedent over any dictionary or extrinsic definition.

The use of the terms “a” and “an” and “the” and similar referents in the context of describing the invention (especially in the context of the following claims) are to be construed to cover both the singular and the plural (i.e., one or more), unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value recited or falling within the range, unless otherwise indicated herein, and each separate value is incorporated into the specification as if it were individually recited.

The term “about” or “approximately” usually means within 5%, or more preferably within 1%, of a given value or range.

The term “biocompatible” as used herein refers to a substance or other material that is non-toxic and/or non-immunogenic. For example, a biocompatible material does not induce a significant immune response or deleterious tissue reaction, e.g., toxic reaction or significant irritation, over time when implanted into or placed adjacent to the biological tissue of a subject.

As used herein, the term “subject” includes any subject who may benefit from being administered a hydrogel or an implantable drug delivery device of the invention. The term “subject” includes animals, e.g., vertebrates, amphibians, fish, mammals, non-human animals, including humans and primates, such as chimpanzees, monkeys and the like. In one embodiment of the invention, the subject is a human. The term “subject” also includes agriculturally productive livestock, for example, cattle, sheep, goats, horses, pigs, donkeys, camels, buffalo, rabbits, chickens, turkeys, ducks, geese and bees; and domestic pets, for example, dogs, cats, caged birds and aquarium fish, and also so-called test animals, for example, hamsters, guinea pigs, rats and mice.

As used herein, the term “subject” includes any subject who may benefit from being administered an adoptive cell therapy, for example, comprising: (i) a tumor-infiltrating lymphocyte (TIL) therapy; (ii) an engineered T cell receptor (TCR) therapy; (iii) a chimeric antigen receptor (CAR) T cell therapy; (iv) a natural killer (NK) cell therapy; or (v) a regulatory T (Treg) cell therapy.

In certain embodiments, a subject can be one who has been previously diagnosed with or otherwise identified as suffering from or having a condition, disease, or disorder. A “subject in need” of treatment for a particular condition can be a subject having that condition, diagnosed as having that condition, or at increased risk of developing that condition relative to a given reference population. In some embodiments, the methods of treatment described herein comprise selecting a subject diagnosed with, suspected of having, or at risk of developing a hematological malignancy or being immunocompromised. In some embodiments, the methods described herein comprise selecting a subject diagnosed with, suspected of having, or at risk of developing a non-malignant disease, for example a non-malignant disease described herein.

As used herein, the term “immune cells” generally refer to resting and/or activated cells of the immune system involved in defending a subject against both infectious disease and foreign materials. Examples of immune cells include, without limitations, white blood cells including, e.g., neutrophils, eosinophils, basophils, lymphocytes (e.g., B-cells, T-cells, and natural killer cells), monocytes, macrophages (including, e.g., resident macrophages, resting macrophages, and activated macrophages); as well as Kupffer cells, histiocytes, dendritic cells, Langerhans cells, mast cells, microglia, and any combinations thereof. In some embodiment, immune cells include derived immune cells, for example, immune cells derived from lymphoid stem cells and/or myeloid stem cells. In some embodiment, immune cells include white blood cells (leukocytes) which are derived from hematopoietic stem cells (HSC) and/or hematopoietic progenitor cells (HPC). In some embodiment, immune cells include hematopoietic stem cells (HSC) and/or hematopoietic progenitor cells (HPC). In some embodiment, immune cells include lymphocytes (T cells, B cells, natural killer (NK) cells) and/or myeloid-derived cells (neutrophil, eosinophil, basophil, monocyte, macrophage, dendritic cells). As used herein, the term “T cell” refers to all types of immune cells expressing CD3 including, without limitation, T-helper cells (CD4+ cells), cytotoxic T-cells (CD8+ cells), T-regulatory cells (Treg), and gamma-delta T cells. As used herein, the term “cytotoxic cell” refer, without limitation, to cells capable of mediating cytotoxicity responses, such as CD8+ T cells, natural-killer (NK) cells, and neutrophils. As used herein, the term “stem cell” generally includes pluripotent or multipotent stem cells. “Stem cells” includes, e.g., embryonic stem cells (ES); mesenchymal stem cells (MSC); induced-pluripotent stem cells (iPS); and committed progenitor cells (hematopoietic stem cells (HSC); bone marrow derived cells, neural progenitor cells, etc.).

As used herein, the term “agent” or “moiety” or “cargo” is defined as any chemical entity that has certain function or activity. An agent or moiety includes, but is not limited to an atom, a chemical group, a small molecule organic compound, an inorganic compound, a nucleoside, a nucleotide, a nucleobase, a sugar, a nucleic acid, an amino acid, a peptide, a polypeptide, a protein, a fusion protein, or a protein complex. The agent or moiety may be detected by methods known in the art. For example, an agent or moiety may be chemiluminescent or fluorescent and can be detected by any suitable chemiluminescent assays known in the art. The function or activity of an agent or moiety may include any physical, chemical, biological, or physiological function or activity. For example, in some embodiments, the agent or moiety may be a radioactive isotope and its activity may include radioactivity. In some other embodiments, the agent or moiety may be cytokine, e.g., an anti-tumor cytokine, such as IL-12, IL-21, and/or TNF-α, and its activity or function may include the anti-tumor activity, the ability to direct T cell differentiation, and/or obtain the desired phenotype.

Generally, the term “treatment” or “treating” is defined as the application or administration of a therapeutic agent to a patient, or application or administration of a therapeutic agent to an isolated tissue or cell line from a patient, said patient having a disease, a symptom of disease or a predisposition toward a disease, with the purpose to cure, heal, alleviate, relieve, alter, remedy, ameliorate, improve or affect the disease, the symptoms of disease or the predisposition toward disease. Thus, treating can include suppressing, inhibiting, preventing, treating, or a combination thereof. Treating refers, inter alia, to increasing time to disease progression, expediting remission, inducing remission, augmenting remission, speeding recovery, increasing efficacy of or decreasing resistance to alternative therapeutics, or a combination thereof. “Suppressing” or “inhibiting”, refers, inter alia, to delaying the onset of symptoms, preventing relapse to a disease, decreasing the number or frequency of relapse episodes, increasing latency between symptomatic episodes, reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, or a combination thereof. In one embodiment the symptoms are primary, while in another embodiment, symptoms are secondary. “Primary” refers to a symptom that is a direct result of a disorder, e.g., diabetes, while, secondary refers to a symptom that is derived from or consequent to a primary cause. Symptoms may be any manifestation of a disease or pathological condition.

As used herein, the term “plurality” intends more than one, and may be used interchangeably, in some embodiments, with the term “population.”

In one embodiment, a plurality of cells refers to at least two cells, for example, at least 3, at least 4, at least 5, at least 6, at least 7, at least 8, at least 9, at least 10, at least 15, at least 20, at least 25, at least 30, at least 35, at least 40, at least 45, at least 50, at least 60, at least 70, at least 80, at least 90, at least 100, at least 125, at least 150, at least 175, at least 200, at least 300, at least 400, at least 500, at least 600, at least 700, at least 800, at least 900, at least 1000, at least 1500, at least 2000, at least 5000, at least 104, at least 105, or more cells. In some embodiments, the plurality of cells are homogenous. In some embodiments, the plurality of cells are heterogeneous. In some embodiments, the plurality of cells are immune cells.

In one embodiment, a plurality of click reagents refers to at least two click reagents, for example, at least 3, at least 4, at least 5, at least 6, at least 7, at least 8, at least 9, at least 10, or more click reagents, e.g., as described herein. In one embodiment, each click reagent in a plurality may be the same or may be different. In some embodiments, each click reagent in a plurality of click reagents may be independently selected from the group consisting of azide, dibenzocyclooctyne (DBCO), transcyclooctene, tetrazine, norbornene, and variants thereof.

In one embodiment, a plurality of click reagents refers to at least two click reagents, for example, a first click reagent and a second click reagent. In one embodiment, a plurality of click reagents refers to at least 3 click reagents, for example, a first click reagent, a second click reagent, and a third click reagent. In one embodiment, a plurality of click reagents refers to at least 4 click reagents, for example, a first click reagent, a second click reagent, a third click reagent, and a fourth click reagent. In one embodiment, a plurality of click reagents refers to at least 5 click reagents, for example, a first click reagent, a second click reagent, a third click reagent, a fourth click reagent, and a fifth click reagent. In one embodiment, a plurality of click reagents refers to at least 6 click reagents, for example, a first click reagent, a second click reagent, a third click reagent, a fourth click reagent, a fifth click reagent, and a sixth click reagent. In one embodiment, a plurality of click reagents refers to at least 7 click reagents, for example, a first click reagent, a second click reagent, a third click reagent, a fourth click reagent, a fifth click reagent, a sixth click reagent, and a seventh click reagent. In one embodiment, a plurality of click reagents refers to at least 8 click reagents, for example, a first click reagent, a second click reagent, a third click reagent, a fourth click reagent, a fifth click reagent, a sixth click reagent, a seventh click reagent, and an eighth click reagent. In one embodiment, a plurality of click reagents refers to at least 9 click reagents, for example, a first click reagent, a second click reagent, a third click reagent, a fourth click reagent, a fifth click reagent, a sixth click reagent, a seventh click reagent, an eighth click reagent, and a ninth click reagent. In one embodiment, a plurality of click reagents refers to at least 10 click reagents, for example, a first click reagent, a second click reagent, a third click reagent, a fourth click reagent, a fifth click reagent, a sixth click reagent, a seventh click reagent, an eighth click reagent, a ninth click reagent, and a tenth click reagent.

By “treatment”, “prevention” or “amelioration” of a disease or disorder is meant delaying or preventing the onset of such a disease or disorder, reversing, alleviating, ameliorating, inhibiting, slowing down or stopping the progression, aggravation or deterioration the progression or severity of a condition associated with such a disease or disorder. In one embodiment, the symptoms of a disease or disorder are alleviated by at least 5%, at least 10%, at least 20%, at least 30%, at least 40%, or at least 50%. Accordingly, as used herein, the term “treatment” or “treating” includes any administration of a compound described herein and includes: (i) preventing the disease from occurring in a subject which may be predisposed to the disease but does not yet experience or display the pathology or symptomatology of the disease; (ii) inhibiting the disease in an subject that is experiencing or displaying the pathology or symptomatology of the diseased (i.e., arresting further development of the pathology and/or symptomatology); or (iii) ameliorating the disease in a subject that is experiencing or displaying the pathology or symptomatology of the diseased (i.e., reversing the pathology and/or symptomatology).

Efficacy of treatment is determined in association with any known method for diagnosing the disorder. Alleviation of one or more symptoms of the disorder indicates that the compound confers a clinical benefit. Any of the therapeutic methods described to above can be applied to any suitable subject including, for example, mammals such as dogs, cats, cows, horses, rabbits, monkeys, and most preferably, humans.

As used herein, the term “cytokine” can refer to any small cell-signaling protein molecule that is secreted by a cell of any type. Cytokines can include proteins, peptides, and/or glycoproteins. Examples of cytokines include, but are not limited to, interleukins, interferons, chemokines, tumor necrosis factors, and colony stimulating factors of immune cell precursors. The term “lymphokines” as used herein generally refers to a subset of cytokines that are produced by a type of immune cell known as a lymphocyte. The term “interleukins” as used herein generally refers to cytokines secreted and/or synthesized by leukocytes and helper CD4+T lymphocytes, and/or through monocytes, macrophages, and/or endothelial cells. In some embodiments, interleukins can be human interleukins including IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, and IL-35. The term “chemokine” as used herein generally refers to a specific class of cytokines that mediates chemoattraction (chemotaxis) between cells. Examples of chemokines include, but are not limited to, CCL family, CXCL family, CX3CL family and XCL family. The term “inflammatory cytokine” as used herein generally includes, without limitation, a cytokine that stimulates an inflammatory response. Examples of inflammatory cytokines include, without limitation, IFN-γ, IL-1, and TNF-α.

In certain embodiments, the cytokine may comprises at least one (e.g., at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, or at least about 100 or more) click moieties, e.g., DBCO moieties.

In some embodiments, the cytokine is coupled to between about 1 and about 100 (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100) click moieties, e.g., DBCO moieties.

In some embodiments, the cytokine is coupled to between about 1 and about 10 (e.g., about 1, about 2, about 3, about 4, about 5, about 6, about 7, about 8, about 9, about 10) click moieties, e.g., DBCO moieties.

In some embodiments, the cytokine is coupled to less than about 10 (e.g., less than about 1, less than about 2, less than about 3, less than about 4, less than about 5, less than about 6, less than about 7, less than about 8, less than about 9, less than about 10) click moieties, e.g., DBCO moieties.

As used herein, the term “glycoprotein” refers to a protein that contains a peptide backbone covalently linked to one or more sugar moieties (e.g., glycans), The sugar moieties may be natural and/or unnatural. The sugar moieties may comprise one or more click moieties, e.g., an azide moiety, a DBCO moiety, a transcyclooctene moiety, a tetrazine moiety, or a norbornene moiety. The sugar moieties may be in the form of monosaccharides, disaccharides, oligosaccharides, and/or polysaccharides. The sugar moieties may comprise a single unbranched chain of sugar residues or may comprise one or more branched chains.

Glycoproteins can contain O-linked sugar moieties and/or N-linked sugar moieties. The polysaccharide may be attached, for example, either via the OH group of serine or threonine (O-glycosylated polypeptide) or via the amide group (NH₂) of asparagine (N-glycosylated polypeptide). The structure and number of sugar moieties attached to a particular glycosylatoin site can be variable, Such sugar moieties (e.g., monosaccharides) may be, for instance, N-acetyl glucosamine (GlcNAc), N-acetyl galactosamine (GalNAc), mannose (Man), galactose (Gal), glucose (Glc), fucose (Fuc), xylose (Xyl), glucuronic acid (GlcA), iduronic acid (IdoA), sialic acid, and/or 5-N-acetylneuraminic acid (Neu5Ac).

In certain embodiments, the monosaccharides, disaccharides, oligosaccharides, and/or polysaccharides may comprise one or more (e.g., at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, or at least about 100 or more) click moieties, e.g., azide moieties.

In certain embodiments, the glycoprotein may comprise one or more (e.g., at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, or at least about 100 or more) click moieties, e.g., azide moieties.

The terms “disease” or “condition” refer to a state of being or health status of a patient or subject capable of being treated with the compositions or methods provided herein. The disease may be a cancer. The disease may be an autoimmune disease. The disease may be an infectious disease, such as a viral disease. In some further instances, “cancer” refers to human cancers and carcinomas, sarcomas, adenocarcinomas, lymphomas, leukemias, etc., including solid and lymphoid cancers, kidney, breast, lung, bladder, colon, ovarian, prostate, pancreas, stomach, brain, head and neck, skin, uterine, testicular, glioma, esophagus, and liver cancer, including hepatocarcinomna, lymphoma, including B-acute lymphoblastic lymphomna, non-Hodgkin's lymphomas (e.g, Burkitt's, Small Cell, and Large Cell lymphomas), Hodgkin's lymphoma, leukemia (including AML, ALL, and CML), or multiple myeloma.

As used herein, the term “autoimmune disease” refers to a disease or condition in which a subject's immune system has an aberrant immune response against a substance that does not normally elicit an immune response in a healthy subject. Examples of autoimmune disorders include multiple sclerosis, type 1 diabetes mellitus, Crohn's disease, rheumatoid arthritis, systemic lupus erythematosus, scleroderma, alopecia areata, antiphospholipid antibody syndrome, autoimmune hepatitis, celiac disease, Graves' disease, Guillain-Barre syndrome, Hashimoto's disease, hemolytic anemia, idiopathic thrombocytopenic purpura, inflammatory bowel disease, ulcerative colitis, inflammatory myopathies, polymyositis, myasthenia gravis, primary biliary cirrhosis, psoriasis, Sjögren's syndrome, vitiligo, gout, atopic dermatitis, acne vulgaris, and autoimmune pancreatitis.

As used herein, the term “infectious disease” refers to an illness caused by a pathogenic biological agent that results from transmission from an infected person, animal, or reservoir to a susceptible host, either directly or indirectly, through an intermediate plant or animal host, vector, or inanimate environment. Infectious pathogens include some viruses, bacteria, fungi, protozoa, multicellular parasites, and aberrant proteins known as prions. In some embodiments, an infectious disease is a viral disease.

### II. Click Chemistry Reagents

In one embodiment, the invention features compositions and reagents for labeling cells, e.g., immune cells, e.g., T cells, using click chemistry reagents. Metabolic glycoengineering of unnatural sugars, azido-sugars for example, provides a facile yet powerful way to introduce chemical groups onto the cell surface in the form of glycoproteins. In some embodiments, for specifically labeling cells in an in vivo environment, these agents can be incorporated into scaffold devices, as described herein. For example, the click chemistry reagents disclosed herein can be incorporated into a device comprising a hydrogel scaffold, that specifically recruits immune cells. Click-labeled cells can be labelled in vitro or in vivo with agents of interest coupled to a counterpart click moiety. In this manner, virtually any agent can be targeted to cells, and covalently coupled to cell surface glycoproteins, using click chemistry. In some embodiments, an agent is delivered to a cell intracellularly. In some embodiments, the agent retains its structural integrity, or function or activity after being delivered to a cell intracellularly.

In some embodiments, click-labeled immune cells, e.g., T cells, can be labelled in vitro or in vivo with agents of interest coupled to a counterpart click moiety. In this manner, virtually any agent, such as a cytokine, e.g., an anti-tumor cytokine, can be targeted to T cells, and covalently coupled to cell surface glycoproteins, using click chemistry.

### Click Functionalized Polymers

In some examples, the present invention provides a click functionalized polysaccharide polymer which is a product of radical-catalyzed polymerization involving a reaction between one or more saccharide monomers. In this radical-catalyzed polymerization, saccharide monomers are polymerized together to form a polysaccharide polymer. Each saccharide monomer involved in the radical-catalyzed polymerization comprises a saccharide molecule; a click reagent that is attached to the saccharide molecule; and a moiety comprising a functional group amenable to radical polymerization that is attached to the saccharide molecule. The product of the radical-catalyzed polymerization is a click functionalized polysaccharide polymer that comprises repeating saccharide units, in which each saccharide unit is attached, e.g., covalently attached, to a click reagent. The click functionalized polymers are described in PCT Application No. PCT/US2019/051621, the entire contents of which are hereby incorporated herein by reference.

In other examples, the present invention also provides a click-functionalized amphiphilic polymer which is a product of radical-catalyzed polymerization involving a reaction between a reagent comprising a hydrophilic portion and one or more saccharide monomers. In this radical-catalyzed polymerization, saccharide monomers are polymerized together to form a polysaccharide polymer, and the hydrophilic portion becomes attached to the polysaccharide polymer. Each saccharide monomer involved in the radical-catalyzed polymerization comprises a saccharide molecule; a click reagent that is attached to the saccharide molecule; and a moiety comprising a functional group amenable to radical polymerization attached to the saccharide molecule. Thus, in some examples, the product of this radical-catalyzed polymerization is a click functionalized polysaccharide polymer that comprises a hydrophilic portion and repeating saccharide units, and in which each saccharide unit is attached, e.g., covalently attached, to a click reagent.

A polymer of the present invention is introduced into a cell, e.g., as a part of a nanoparticle, the polymer is subjected to hydrolysis, resulting in release inside the cell of individual saccharide monomers attached to a click-reagent. The individual saccharide monomers attached to a click reagent are then subjected to metabolic glycoengineering inside the cell, resulting in incorporation of the saccharide monomers attached to a click reagent into post-translational modifications of, inter alia, proteins of the plasma membrane. The click reagents are then displayed on the cell surface as the proteins span the plasma membrane. As a result, the cell surface becomes labeled with a click reagent.

Any saccharide molecule amenable to metabolic glycoengineering inside a cell may be used to prepare saccharide monomers for preparing click functionalized polymers of the invention. In certain embodiments, the saccharide molecule may be selected from the group consisting of mannose, galactose, fucose and sialic acid. In one specific embodiment, the saccharide molecule may be mannose.

In some examples, in the saccharide monomers, the click reagent may be attached to the saccharide molecule at the C2 position of the sugar moiety. For example, the click reagent may be an azide, and the saccharide molecule may be a mannose, e.g., an acetylated mannose. As illustrated below, an azide may be attached at the C2 position of an acetylated mannose:

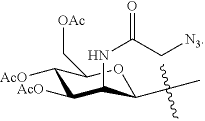

The term “click reagent”, which may be used herein interchangeably with the term “click chemistry reagent” and “click moiety”, refers to a reagent that can rapidly and selectively react (“click”) with its counterpart click reagent under mild conditions in aqueous solution. The mild conditions may include any one of neutral pH, aqueous solution and ambient temperature, with low reactant concentrations. Any suitable click reagent may be used in the context of the present invention. Exemplary click pair reagents are well known to one of skill in the art and include, but are not limited to, moieties that comprise azide and dibenzocyclooctyne (DBCO), tetrazine and transcyclooctene, and tetrazine and norbornene, with the structures illustrated below.

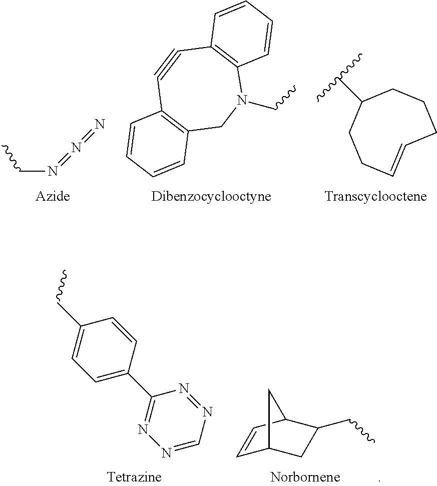

In some embodiments, the click reagent may be an azide. The term “azide” or “azide moiety”, as used herein, includes molecules that comprise an azide moiety as shown above. In some examples, azide may be attached to the saccharide molecule with a suitable spacer moiety. In a specific example, the spacer moiety comprises an aminocarbonyl linkage. The term “aminocarbonyl” or “amide”, as used herein, includes compounds or moieties which contain a nitrogen atom which is bonded to the carbon of a carbonyl or a thiocarbonyl group. This term includes “alkaminocarbonyl” or “alkylaminocarbonyl” groups wherein alkyl, alkenyl, aryl or alkynyl groups are bound to an amino group bound to a carbonyl group. In one specific example, the azide moiety and the spacer moiety may be represented by the following structure:

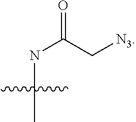 [SMILES: *NC(=O)CN=[N+]=[N-]]

A counterpart click reagent for an azide is dibenzocyclooctyne (DBCO). In some embodiments, the click reagent may be DBCO. As used herein, the term “DBCO” or “DBCO moiety” includes molecules that may comprise a DBCO moiety as shown above. In some examples, DBCO is attached to the saccharide molecule with a suitable spacer moiety, e.g., comprising an aminocarbonyl or an alkylamino linkage. The term “alkylamino”, as used herein, includes moieties wherein a nitrogen atom is covalently bonded to at least one carbon or heteroatom and to at least one alkyl group. This term also includes “dialkylamino”, wherein the nitrogen atom is bound to at least two alkyl groups.

In some embodiments, the click reagent may be tetrazine. As used herein, the term “tetrazine” or “tetrazine moiety” includes molecules that may comprise a tetrazine moiety as shown above. In some examples, transcyclooctene is attached to the saccharide molecule with a suitable spacer moiety, e.g., comprising an aminocarbonyl or an alkylamino linkage. Exemplary tetrazine moieties suitable within the context of the present invention include, but are not limited to, the structures shown below (see, e.g., Karver et al., (2011) Bioconjugate Chem. 22:2263-2270, and WO 2014/065860, the entire contents of each of which are hereby incorporated herein by reference):

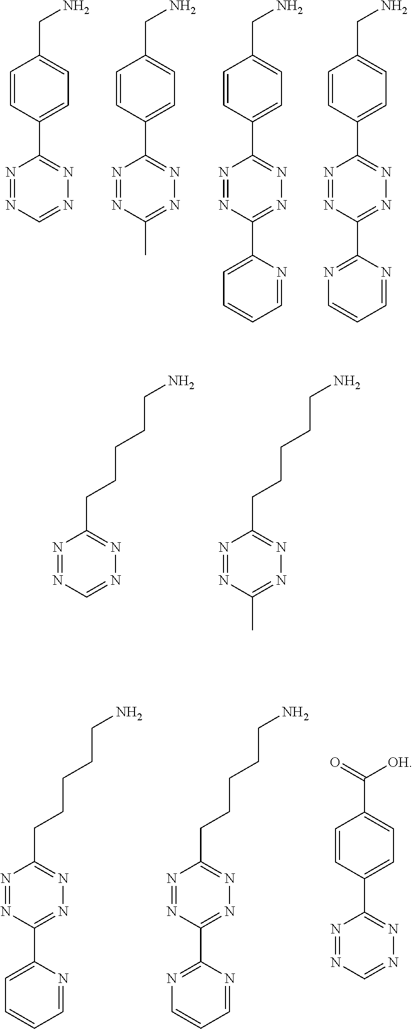

In other examples, exemplary tetrazines that may be used in the context of the present invention are described in U.S. Pat. No. 8,236,949, the entire contents of which are hereby incorporated herein by reference.

One of the counterpart click reagent for a tetrazine is transcyclooctyne. In some embodiments, the click reagent in the context of the present invention may be transcyclooctene. As used herein, the term “transcyclooctene” or “transcyclooctene moiety” includes molecules that may comprise a transcyclooctene moiety as shown above. In some examples, transcyclooctene is attached to the saccharide molecule with a suitable spacer moiety, e.g., comprising an aminocarbonyl or an alkylamino linkage. Exemplary transcyclooctenes that may be used in the context of the present invention include the transyclooctynes described, e.g., in U.S. Pat. No. 8,236,949, the entire contents of which are hereby incorporated herein by reference.

Another counterpart reagent for tetrazine is norbornene (Nb). In some embodiments, the click reagent in the context of the present invention may be norbornene. As used herein, the terms “norbornene” and “norbornene moieties” include but are not limited to the norbornene moiety as shown above, including a moiety comprising norbornadiene and norbornene groups. In some examples, norbornene is attached to the saccharide molecule with a suitable spacer moiety, e.g., comprising an aminocarbonyl or an alkylamino linkage.

In addition to the click reagent, the saccharide monomer may also comprise a moiety comprising a functional group amenable to radical polymerization. The presence of such a moiety in the saccharide monomer provides the means to polymerize the saccharide moieties, thereby forming a click functionalized polymer of the invention. The moiety comprising a functional group amenable to radical polymerization may comprise a double bond. For example, the moiety comprising a functional group amenable to radical polymerization may comprise an acrylate or a methacrylate. In one specific example, the moiety comprising a functional group amenable to radical polymerization comprises an acrylate. In another specific example, the moiety comprising a functional group amenable to radical polymerization comprises a methacrylate.

The moiety comprising a functional group amenable to radical polymerization may be attached to the saccharide molecule, e.g., mannose, galactose, fucose or sialic acid, at the C1 position, the C3 position, the C4 position or the C5 position of the saccharide molecule. In one specific embodiment, the moiety comprising a functional group amenable to radical polymerization is attached to the saccharide molecule at the C1 position.

Illustrated below is an exemplary saccharide monomer comprising mannose as the saccharide molecule, an azide as the click reagent attached at the C2 position of the mannose and the acrylate as the moiety comprising a functional group amenable to radical polymerization attached at the C1 position. The exemplary saccharide monomer is further acetylated at the C3, C4 and C5 positions of the mannose:

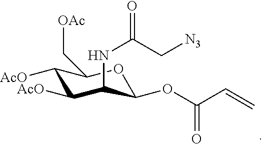

The saccharide monomer used in the radical-catalyzed polymerization to produce the polymers of the present invention may further comprise one or more hydrolysable substituents at any position that is not occupied by the click reagent or moiety comprising a functional group amenable to radical polymerization. For example, a hydrolysable substituent may be present at the C1 position, the C3 position, the C4 position or C5 position of the saccharide monomer. In some examples, the hydrolysable substituent contributes to the hydrophobicity of the polymer, but, once inside the cell, may be hydrolyzed and converted to a hydroxyl group. In some example, the hydrolysable substituent is represented by formula (1):

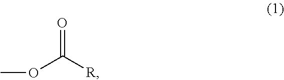

wherein R is alkyl. In a specific example, R is methyl.

The term “alkyl”, as used herein, includes saturated aliphatic groups, including straight-chain alkyl groups (e.g., methyl, ethyl, propyl, butyl, pentyl, hexyl, heptyl, octyl, nonyl, decyl, etc.), branched-chain alkyl groups (e.g., isopropyl, tert-butyl, isobutyl, etc.). The term alkyl also includes alkyl groups which can further include oxygen, nitrogen, sulfur or phosphorous atoms replacing one or more carbons of the hydrocarbon backbone. In some examples, a straight chain or branched chain alkyl may have 6 or fewer carbon atoms in its backbone (e.g., C₁-C₆ for straight chain, C₃-C₆ for branched chain), and more preferably 4 or fewer. The term “C₁-C₆” includes alkyl groups containing 1 to 6 carbon atoms.

In some examples, the click functionalized polysaccharide polymers of the present invention may comprise 10 to 1000 saccharide units, i.e., 10 to 1000 saccharide monomers attached together to form the click functionalized polysaccharide polymer. For example, the polymers of the invention may comprise 20 to 500, 100 to 500 or 200 to 600 saccharide units. In one specific example, the polymer of the invention may comprise 10-50 saccharide units, e.g., 25 saccharide units. In another specific example, the polymer of the invention may comprise 300-500 saccharide units, e.g., 400 saccharide units. In one specific embodiment, the polymer of the invention may comprise the structure of formula (2):

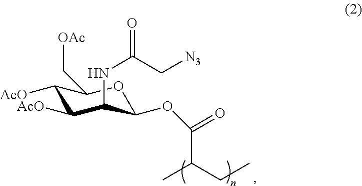

wherein n is a number between 10 and 1000.

In some examples, the click functionalized polysaccharide polymer of the present invention may further comprise a hydrophilic portion. The hydrophilic portion may be attached to the repeating saccharide units in which each saccharide unit is attached, e.g., covalently attached, to a click reagent. The hydrophilic portion may comprise a hydrophilic polymer, such as polyethylene oxide (PEG). In some examples, the PEG may comprise between 20 and 450 PEG units, e.g., about 100 to about 150 PEG units. In some examples, the PEG may have an average molecular weight of about 500 to about 20,000 Daltons, e.g., about 2,000 and about 10,000 Dalton. In one example, the PEG has an average molecular weight of about 5,000 Daltons.

In some examples, the click functionalized polysaccharide polymer of the invention comprising a hydrophilic portion may comprise the structure of formula (3):

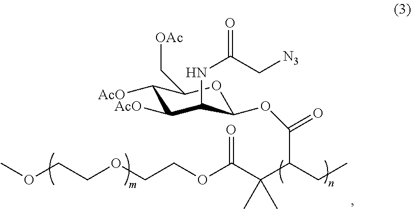

wherein n is a number between 10 and 1000; and m is a number between 45 and 200.

The polymers of the invention are produced by subjecting saccharide monomers as described above and, optionally, the hydrophilic portion, to a radical-catalyzed polymerization. In some examples, the radical-catalyzed polymerization may be reversible addition-fragmentation chain transfer (RAFT) polymerization. The RAFT polymerization involves conventional free radical polymerization of a substituted monomer in the presence of a suitable chain transfer agent (RAFT agent or CTA), which mediate the polymerization via a reversible chain-transfer process.

Any suitable RAFT reagent may be used in the context of the present invention. Exemplary RAFT agents may be found, e.g., in the SIGMA-ALDRICH catalog and may comprise a thiocarbonate moiety, a dithiocarbamate moiety or a dithiobenzoate moiety. In one specific example, the RAFT agent may comprise a thiocarbonate moiety, e.g., 2-(dodecylthiocarbonothioylthio)-2-methylpropionate.

In another specific example, the RAFT agent may comprise poly(ethylene glycol) methyl ether 2-(dodecylthiocarbonothioylthio)-2-methylpropionate. In this example, when the RAFT agent participates in the radical-catalyzed polymerization, the poly(ethylene glycol) portion of the RAFT agent becomes attached to the resulting click functionalized polysaccharide polymer and becomes the hydrophilic portion of the polymer. An exemplary product of the RAFT polymerization that comprises the use of poly(ethylene glycol) methyl ether 2-(dodecylthiocarbonothioylthio)-2-methylpropionate as the RAFT agent is the structure of formula (4):

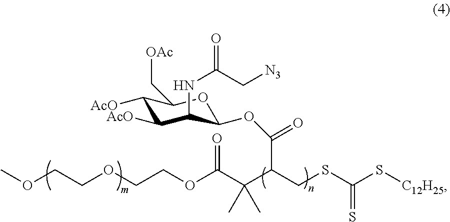

wherein n is a number between 10 and 1000; and m is a number between 45 and 200.

### Nanoparticles

The present invention also provides nanoparticles, e.g., unnatural azido-sugar nanoparticles, for labeling cells with a click reagent. The nanoparticles may comprise the click functionalized polysaccharide polymer of the invention as described above.

In some examples, the nanoparticle may be self-assembling, i.e., may spontaneously form when click functionalized polysaccharide polymer of the invention, once prepared, is exposed to certain conditions, such as an aqueous solvent or a physiological pH, or when the click functionalized polysaccharide polymer of the invention is subjected to nanoprecipitation. Scheme 1 below illustrates preparation of an exemplary nanoparticle of the invention starting from synthesis of a click functionalized polysaccharide polymer using RAFT polymerization. The RAFT reagent used in the RAFT polymerization is poly(ethylene glycol) methyl ether 2-(dodecylthiocarbonothioylthio)-2-methylpropionate. The saccharide monomer used in the RAFT polymerization to produce the click functionalized polysaccharide polymer is Ac₃ManAz-acrylate. The Ac₃ManAz-acrylate comprises mannose as the saccharide molecule which is functionalized at the C1 position with an azide as the click reagent and at the C2 positon with an acrylate as the moiety comprising a functional group amenable to radical polymerization. The Ac₃ManAz-acrylate further comprises acetyl groups at the C3, C4 and C5 positions as the hydrolysable substituents. The resulting polymer also comprises PEG₅ₖ (or PEG having an average molecular weight of about 5000 Daltons) as the hydrophilic portion. In the last step, a nanoparticle is produced by subjecting the click functionalized polysaccharide polymer of the invention to nanoprecipitation.

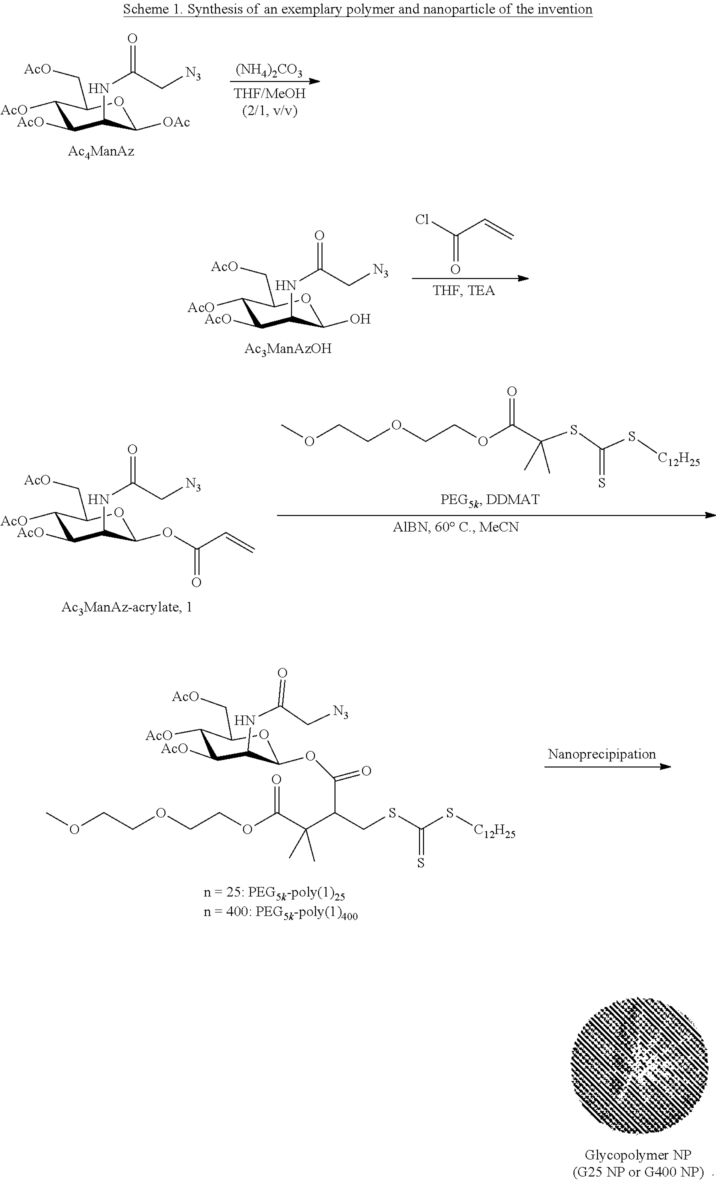

In other examples, the nanoparticle of the invention does not comprise a click functionalized polysaccharide polymer. Rather, the nanoparticle of the invention may comprise a saccharide molecule, e.g., a monomeric saccharide molecule, attached to a click reagent. For example, the saccharide molecule may be selected from the group consisting of mannose, galactose, fucose and sialic acid. In one specific example, the saccharide molecule is mannose.

The click reagent may be attached to the saccharide molecule at the C2 position and may comprise any of the click reagents as described above for saccharide monomers. The saccharide molecule may also comprise one or more hydrolysable substituents at the C1, C3, C4 and/or C5 positions of the saccharide molecule as described above for saccharide monomers.

The nanoparticle useful in the context of the present invention may be selected from the group consisting of a carbon-based nanoparticle, a ceramic nanoparticle, a metal nanoparticle, a semiconductor nanoparticle, a polymeric nanoparticle and a lipid-based nanoparticle. In one specific example, the nanoparticle may be a lipid-based nanoparticle, e.g., a liposome or a micelle. In another specific example, the nanoparticle useful in the context of the present invention may be a semiconductor nanoparticle, e.g., a silica nanoparticle.

### Exemplary Poly(Azido-Sugar) and Nanoparticles for Metabolic Labeling of Cells

Poly(azido-sugar) can label a variety of cells with azido groups. Exemplary methods to prepare tetraacetyl-N-azidoacetylmannosamine (Ac₄ManAz) and poly (azido-sugar) have been described in, for example, International Patent Applications PCT/US2019/051621 and PCT/US2021/015912, each of which are hereby incorporated herein by reference. Briefly, Ac₄ManAz may be synthesized following the reported procedure (Wang, H. et al. Selective in vivo metabolic cell-labeling-mediated cancer targeting. Nature Chemical Biology 13, 415-424 (2017)).

An exemplary method to prepare poly(azido-sugar) is described in Example 1 of International Patent Applications PCT/US2021/015912. Specifically, as described in International Patent Applications PCT/US2021/015912, to prepare poly(azido-sugar), the C1 site of Ac₄ManAz was functionalized with an acrylate bond, followed by reversible addition-fragmentation chain transfer (RAFT) polymerization using poly(ethylene glycol) methyl ether 2-(dodecylthiocarbonothioylthio)-2-methylpropionate as the RAFT agent and azobisisobutyronitrile as the initiator to yield poly(azido-sugar)ₙ (n=400 (G400)). Briefly, Ac₄ManAz (1 mmol) was dissolved in methanol/tetrahydrofuran (1/2, v/v), followed by the addition of ammonium carbonate (1.2 mmol). The reaction mixture was stirred at room temperature for 24 hours. After removal of the solvent under reduced pressure, the crude product was purified by silica gel column chromatography to yield Ac₃ManAzOH. Ac₃ManAzOH (1.0 mmol) was then dissolved in dry dichloromethane, followed by the addition of acryloyl chloride (3.0 mmol) and triethylamine (1.0 mmol). The reaction mixture was stirred at room temperature for 24 hours. After removal of the solvent and residual acryloyl chloride, the crude product was redissolved in dichloromethane, washed with deionized water for three times, and dried to yield Ac₃ManAzAL. Ac₃ManAzAL (1.0 mmol), azobisisobutyronitrile (AIBN, 0.008 or 0.0005 mmol), and poly(ethylene glycol) methyl ether 2-(dodecylthiocarbonothioylthio)-2-methylpropionate (PEG DDMAT, 0.04 or 0.0025 mmol) were dissolved in anhydrous DMF, followed by three freeze-thaw cycles and stirring at 65° C. for 48 hours. Poly(azido-sugar) (G400) was obtained via precipitation in cold diethyl ether, washed with diethyl ether for three times, and dried under reduced pressure. G400 NP were then prepared via nanoprecipitation of G400. Briefly, G400 polymer was dissolved in DMF at a concentration of 40 mg/mL, and dropwise added to ultrapure water (20-fold volume) upon vigorous stirring. After stirring for 4 hours, G400 NP solution was dialyzed against deionized water for 48 hours, sterilized, and then stored at 4° C. for use.

### III. Compositions and Methods for Labeling Cells

In some embodiments, the present invention also provides a method for labeling a cell with a click reagent that comprises contacting the cell with the click functionalized polysaccharide polymer of the invention as described above. In other embodiments, the present invention also provides a method for labeling a cell with a click reagent that comprises contacting the cell with an unnatural sugar and/or an unnatural sugar nanoparticle of the invention as described above. Contacting the cell with the unnatural sugars, polymers, and/or nanoparticles of the invention can take place in vitro, ex vivo, in situ, or in vivo. The compositions and methods for labeling cells have been described in detail in International Patent Applications PCT/US2019/051621 and PCT/US2021/015912, the entire contents of which are incorporated herein by reference.

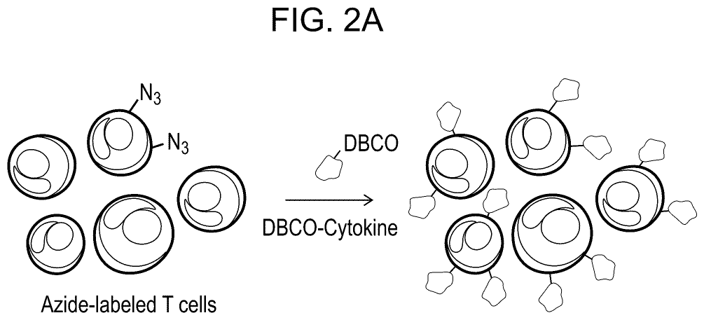

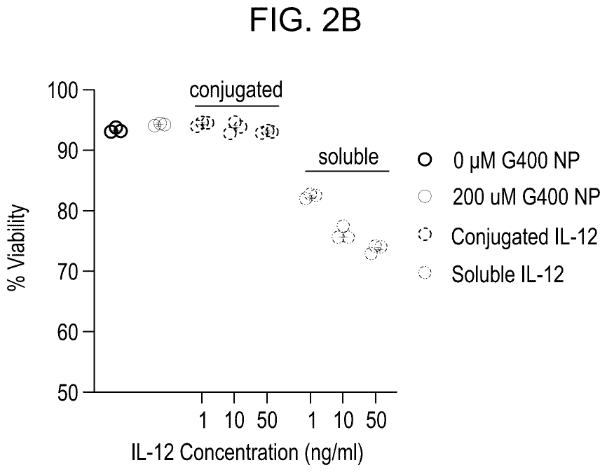

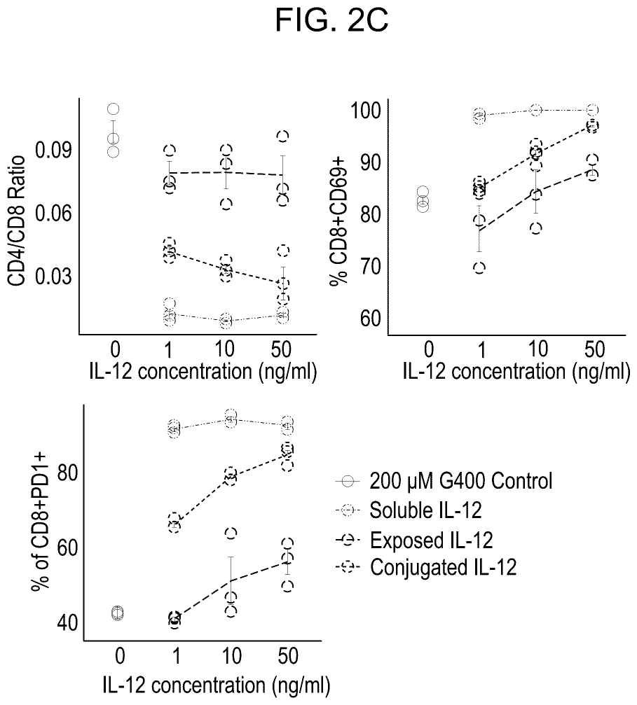

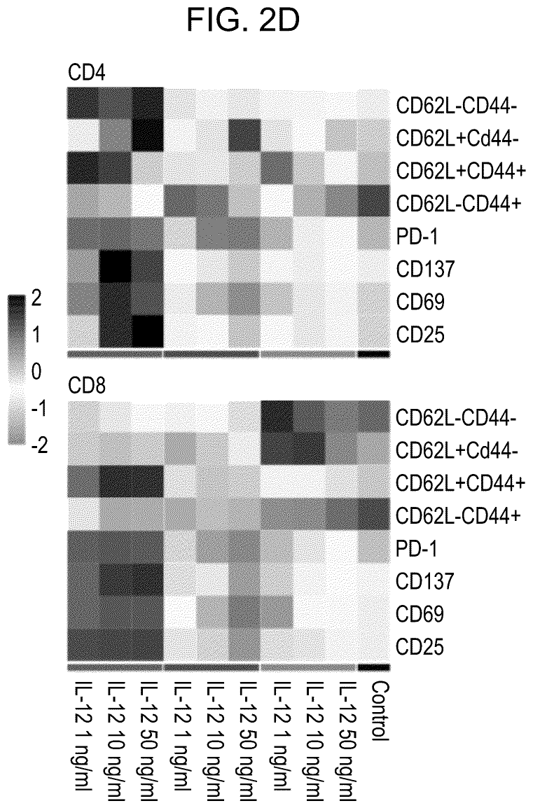

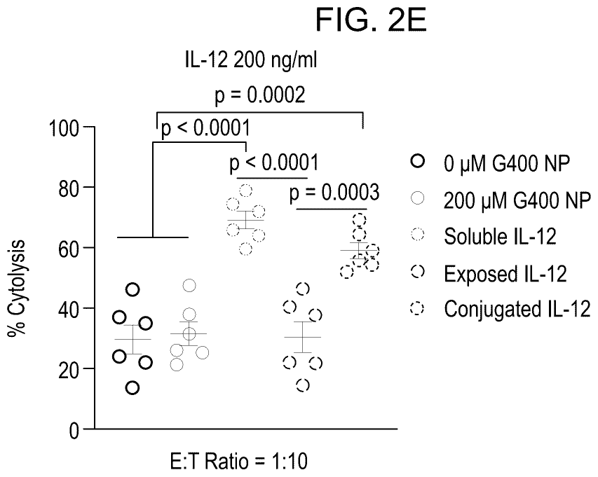

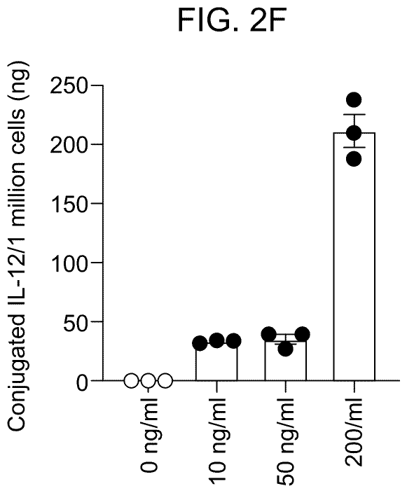

The foregoing unnatural sugar, polymer, and/or nanoparticle compositions can be used to metabolically label the surface of cells with click chemistry reagents. Click chemistry reagents including sugar moieties, and nanoparticles comprising the click chemistry reagents as described herein, can enter cells by endocytosis, and subsequently disassemble and degrade by hydrolysis or enzymatic degradation. The released sugar-click reagent is metabolically processed, and is presented on the surface of the cell in the form of a glycoprotein. This process is illustrated schematically for the exemplary embodiment of azido-sugar nanoparticles in FIGS. 2E and 2F of PCT/US2019/051621.

Preferably, cells are contacted with an effective amount of the click chemistry reagent. In some embodiments, the effective amount is an amount sufficient to metabolically label at least 10% of cell surface glycoproteins with a click moiety, e.g., an azide moiety, a DBCO moiety, a transcyclooctene moiety, a tetrazine moiety, or a norbornene moiety. The amount of a click chemistry reagent needed to metabolically label cells can readily be determined for each reagent and each cell type. In exemplary embodiments, the click reagent is provided to cells at a concentration of 1 nM to 1 μM. In other exemplary embodiments, the click reagent is provided to cells at a concentration of 1 μM to 1 mM. In other exemplary embodiments, the click reagent is provided to cells at a concentration of 1 mM to 1 μM.

In some embodiments, the amount of the chemistry reagent is sufficient to label about 5% to about 100% of the cell surface glycoproteins with the click moiety. In certain embodiments, the amount of the chemistry reagent is sufficient to label about 5%, about 10%, about 20%, about 30%, about 40%, about 50%, about 60%, about 70%, about 80%, about 90%, or about 99% of the cell surface glycoproteins. The intermediaries and ranges between the recited values are contemplated as part of the invention. The labeling efficiency can be readily determined in any methods known in the art. For example, the labeling efficiency can be determined by measuring the percentage of the total cell surface glycoproteins that can react to the counter click reagent using any suitable fluorescence assays and/or immunoassays.

In certain embodiments, at least about 10⁷ to about 10⁸ click moieties, e.g., azide, are used to label a single cell. In some embodiments, about 1×10⁷, about 2×10⁷, about 3×10⁷, about 4×10⁷, about 5×10⁷, about 6×10⁷, about 7×10⁷, about 8×10⁷, about 9×10⁷, or about 10⁸ click moieties, or more than about 10⁸ click moieties are used to label a single cell.

Intermediaries and ranges between the recited values are contemplated as part of the invention.

Virtually any cell type can be labeled with a click reagent in this manner. For example, this method can be used to label an epithelial cell, a fibroblast cell, a neuronal cell, an endothelial cell, and/or an immune cell with a click reagent. In an exemplary embodiment, the method is used to label immune cells, for example, dendritic cells, T cells, macrophages, B cells, or neutrophils. In one embodiment, the cells are CAR-T cells. In another embodiment, the cells are Sipuleucel-T, a mixture of antigen presenting cells used as an immunotherapy agent. In other exemplary embodiments, the click chemistry reagents disclosed herein can be used to label leukocytes, e.g. peripheral blood leukocytes, spleen leukocytes, lymph node leukocytes, hybridoma cells, T cells (cytotoxic/suppressor, helper, memory, naive, and primed), B cells (memory and naive), monocytes, macrophages, granulocytes (basophils, eosinophils, and neutrophils), natural killer cells, natural suppressor cells, thymocytes, and dendritic cells; cells of the hematopoietic system, e.g. hematopoietic stem cells (CD34+), proerythroblasts, normoblasts, promyelocytes, reticulocytes, erythrocytes, pre-erythrocytes, myeloblasts, erythroblasts, megakaryocytes, B cell progenitors, T cell progenitors, thymocytes, macrophages, mast cells, and thrombocytes; stromal cells, e.g. adipocytes, fibroblasts, adventitial reticular cells, endothelial cells, undifferentiated mesenchymal cells, epithelial cells including squamous, cuboid, columnar, squamous keratinized, and squamous non-keratinized cells, and pericytes; cells of the skeleton and musculature, e.g. myocytes (heart, striated, and smooth), osteoblasts, osteoclasts, osteocytes, synoviocytes, chondroblasts, chondrocytes, endochondral fibroblasts, and perichonondrial fibroblasts; cells of the neural system, e.g., neural crest cells, astrocytes (protoplasmic and fibrous), microglia, oligodendrocytes, and neurons; cells of the digestive tract, e.g. parietal, zymogenic, argentaffin cells of the duodenum, polypeptide-producing endocrine cells (APUD), islets of langerhans (alpha, beta, and delta), hepatocytes, and kupfer cells; cells of the skin, e.g. keratinocytes, langerhans, and melanocytes; cells of the pituitary and hypothalamus, e.g. somatotropic, mammotropic, gonadotropic, thyrotropic, corticotropin, and melanotropic cells; cells of the adrenals and other endocrine glands, e.g., thyroid cells (C cells and epithelial cells); adrenal cells; cells of the reproductive system, e.g., oocytes, spermatozoa, leydig cells, embryonic stem cells, amniocytes, blastocysts, morulas, and zygotes; and tumor cells.

In one embodiment, the click chemistry reagents disclosed herein are used to label immune cells, e.g., dendritic cells, T cells, CAR-T cells, B cells, NK cells, monocytes, and macrophages.

In an exemplary embodiment, the cells are contacted with the reagent for a period of time sufficient for cells to take up the reagent by endocytosis. The period of time sufficient for the cell to take up the click chemistry reagent can be determined empirically, for example, by microscopy, flow cytometry, and other standard techniques. In exemplary embodiments, the period of time sufficient for the cell to take up the click chemistry reagent is about 3 hours, about 6 hours, about 12 hours, about 24 hours, about 48 hours, about 72 hours, about 96 hours, about 120 hours, or more. In other embodiments, the period of time sufficient for the cell to take up the click chemistry reagent is about 24-120 hours, about 48-96 hours, or about 48-72 hours. Intermediaries and ranges between the recited values are contemplated as part of the invention.

Metabolic processing of the click chemistry reagent occurs inside the cell, whereby the sugar moiety is partially degraded and incorporated into glycoproteins, which are then displayed on the cell surface. After processing, the cells contain cell surface proteins which comprise carbohydrate molecules labeled with the click moiety.

Accordingly, in another aspect, the invention provides a cell comprising a cell surface glycoprotein, wherein the glycoprotein comprising a carbohydrate covalently linked to a click reagent. In exemplary embodiments, the click reagent comprises azide, dibenzocyclooctyne (DBCO), transcyclooctene, tetrazine and/or norbornene, or variants thereof. In some embodiments, the cell is an isolated cell. In some embodiments, the cell is an epithelial cell, a fibroblast cell, a neuronal cell, an endothelial cell, or an immune cell. In exemplary embodiments, the cell is an immune cells, for example, a T cell, a macrophage, a B cell, a dendritic cell, or a neutrophil. In one embodiment, the cell is a CAR-T cell. In another embodiment, the cell is Sipuleucel-T, a mixture of antigen presenting cells used as an immunotherapy agent. In other exemplary embodiments, the cell is a cell type selected from leukocytes, e.g. peripheral blood leukocytes, spleen leukocytes, lymph node leukocytes, hybridoma cells, T cells (cytotoxic/suppressor, helper, memory, naive, and primed), B cells (memory and naive), monocytes, macrophages, granulocytes (basophils, eosinophils, and neutrophils), natural killer cells, natural suppressor cells, thymocytes, and dendritic cells; cells of the hematopoietic system, e.g. hematopoietic stem cells (CD34+), proerythroblasts, normoblasts, promyelocytes, reticulocytes, erythrocytes, pre-erythrocytes, myeloblasts, erythroblasts, megakaryocytes, B cell progenitors, T cell progenitors, thymocytes, macrophages, mast cells, and thrombocytes; stromal cells, e.g. adipocytes, fibroblasts, adventitial reticular cells, endothelial cells, undifferentiated mesenchymal cells, epithelial cells including squamous, cuboid, columnar, squamous keratinized, and squamous non-keratinized cells, and pericytes; cells of the skeleton and musculature, e.g. myocytes (heart, striated, and smooth), osteoblasts, osteoclasts, osteocytes, synoviocytes, chondroblasts, chondrocytes, endochondral fibroblasts, and perichonondrial fibroblasts; cells of the neural system, e.g. neural crest cells, astrocytes (protoplasmic and fibrous), microglia, oligodendrocytes, and neurons; cells of the digestive tract, e.g. parietal, zymogenic, argentaffin cells of the duodenum, polypeptide-producing endocrine cells (APUD), islets of langerhans (alpha, beta, and delta), hepatocytes, and kupfer cells; cells of the skin, e.g. keratinocytes, langerhans, and melanocytes; cells of the pituitary and hypothalamus, e.g. somatotropic, mammotropic, gonadotropic, thyrotropic, corticotropin, and melanotropic cells; cells of the adrenals and other endocrine glands, e.g. thyroid cells (C cells and epithelial cells); adrenal cells; cells of the reproductive system, e.g. oocytes, spermatozoa, leydig cells, embryonic stem cells, amniocytes, blastocysts, morulas, and zygotes; and tumor cells.

In an exemplary embodiment, the invention provides an isolated immune cell comprising a cell surface glycoprotein, wherein the glycoprotein comprising a carbohydrate covalently linked to an azide.

The click-labeled cells disclosed herein can, in some embodiments, be administered to a subject, e.g., a mammalian subject, such as a murine subject, a primate subject, or a human subject.

In some embodiments, click-labeled cells are administered to a subject as part of a treatment regimen. For example, in some embodiments, click-labeled cells can be administered to a subject to enhance an adoptive cell therapy, for example, comprising: (i) a tumor-infiltrating lymphocyte (TIL) therapy; (ii) a engineered T cell receptor (TCR) therapy; (iii) a chimeric antigen receptor (CAR) T cell therapy; (iv) a natural killer (NK) cell therapy; or (v) a regulatory T (Treg) cell therapy.

In some embodiments, the click-labeled cells are administered to a subject embedded in a device, e.g., a polymer scaffold device. Accordingly, in one aspect, the invention provides a device comprising a polymer scaffold, and cells comprising a cell surface glycoprotein, wherein the glycoprotein comprises a carbohydrate covalently linked to a click reagent. Exemplary polymer scaffolds suitable for delivery of click-labeled cells to a subject include hydrogel scaffolds and cryogel scaffolds. In one embodiment, cells are delivered in an alginate scaffold. The scaffold can be porous or non-porous. In some embodiments, the scaffold is initially non-porous, but forms pores in situ after administration to a subject. Non-limiting examples of scaffolds that can be used to deliver cells are described in US 2014/0079752 A1, published Apr. 12, 2012; US 2016/0271298 A1, published Sep. 22, 2016; and WO 2018/026884 A1, published Feb. 8, 2018. The entire contents of each of the foregoing publications are incorporated herein by reference. Additional features of devices and scaffolds that can be used to deliver cells to a subject are described herein.

In some aspects, the invention provides compositions and methods for labeling cells with a click reagent of the invention in vivo. In some embodiments, the invention provides a method of labeling cells with a click reagent in vivo, comprising administering a click reagent disclosed herein to a subject. In exemplary embodiments, the click reagent is provided as an unnatural sugar, a polymer, and/or a nanoparticle, as described herein. In some embodiments, the click reagent, unnatural sugar, polymer, and/or nanoparticle can be incorporated into a polymer scaffold device. Devices suitable for the incorporation of click reagents are disclosed herein. Such devices can be used to label cells that contact the scaffold with click reagents. In some embodiments, the devices described herein can be used to label immune cells, e.g., a lymphocytes, e.g., B-cells, T-cells, natural killer (NK) cells, regulatory T (Treg) cells, or a combination thereof, with click reagents.

Accordingly, in one aspect, the invention provides a device comprising a polymer scaffold and a click reagent. A number of biomaterial scaffolds are available that allow the migration of cells into an out of the scaffold in vivo. Incorporation of the click reagents of the invention into such scaffolds provides a platform for contacting cells in vivo with the click reagents, thereby allowing metabolic labeling of cells that contact the scaffold in vivo. Labeling of specific cell types in vivo can be achieved by modifying the device to promote recruitment of the desired cells to the scaffold. For example, the device can contain chemoattractants that promote recruitment of specific cell types to the scaffold in vivo. In some embodiments, the click reagents of the invention are formatted as an unnatural sugar. In some embodiments, the click reagents of the invention are formatted as a polymer, e.g., a click functionalized polysaccharide polymer, or as a nanoparticle, as described herein. The device scaffolds suitable for metabolically labeling cells are described in PCT/US2019/051621, incorporated herein by reference.

In some embodiments, cells are labeled with a click reagent in vitro and administered to a subject in the absence of a scaffold. The agent coupled to a counterpart click reagent is administered to the subject separately or together with the click-agent labeled cell. The click reaction between the click reagents allows the agent to be specifically or selectively targeted to the click-reagent labeled cell.

Exemplary features of the devices of the present disclosure are provided below.

### Device Scaffold

The devices of the present disclosure can comprise a scaffold, e.g., a polymer scaffold. The scaffold can comprise one or more biomaterials. Preferably, the biomaterial is a biocompatible material that is non-toxic and/or non-immunogenic.

The scaffold can comprise biomaterials that are non-biodegradable or biodegradable. In certain embodiments, the biomaterial can be a non-biodegradable material. Exemplary non-biodegradable materials include, but are not limited to, metal, plastic polymer, or silk polymer. In certain embodiments, the polymer scaffold comprises a biodegradable material. The biodegradable material may be degraded by physical or chemical action, e.g., level of hydration, heat, oxidation, or ion exchange or by cellular action, e.g., elaboration of enzyme, peptides, or other compounds by nearby or resident cells. In certain embodiments, the polymer scaffold comprises both non-degradable and degradable materials.

In some embodiments, the scaffold composition can degrade at a predetermined rate based on a physical parameter selected from the group consisting of temperature, pH, hydration status, and porosity, the cross-link density, type, and chemistry or the susceptibility of main chain linkages to degradation or it degrades at a predetermined rate based on a ratio of chemical polymers. For example, a high molecular weight polymer comprised of solely lactide degrades over a period of years, e.g., 1-2 years, while a low molecular weight polymer comprised of a 50:50 mixture of lactide and glycolide degrades in a matter of weeks, e.g., 1, 2, 3, 4, 6, 10 weeks. A calcium cross-linked gels composed of high molecular weight, high guluronic acid alginate degrade over several months (1, 2, 4, 6, 8, 10, 12 months) to years (1, 2, 5 years) in vivo, while a gel comprised of low molecular weight alginate, and/or alginate that has been partially oxidized, will degrade in a matter of weeks.

In certain embodiments, one or more compounds disclosed herein are covalently or non-covalently linked or attached to the scaffold composition. In various embodiments, one or more compounds disclosed herein is incorporated on, into, or present within the structure or pores of, the scaffold composition.

In some embodiments, the scaffolds comprise biomaterials that are modified, e.g., oxidized or reduced. The degree of modification, such as oxidation, can be varied from about 1% to about 100%. As used herein, the degree of modification means the molar percentage of the sites on the biomaterial that are modified with a functional group. For example, the degree of modification can be about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%. It is intended that values and ranges intermediate to the recited values are also intended to be part of this invention. Exemplary modified biomaterials, e.g., hydrogels, include, but not limited to, reduced-alginate, oxidized alginate, MA-alginate (methacrylated alginate) or MA-gelatin.

Exemplary biomaterials suitable for use as scaffolds in the present invention include glycosaminoglycan, silk, fibrin, MATRIGEL®, poly-ethyleneglycol (PEG), polyhydroxy ethyl methacrylate, polyacrylamide, poly (N-vinyl pyrolidone), (PGA), poly lactic-co-glycolic acid (PLGA), poly e-carpolactone (PCL), polyethylene oxide, poly propylene fumarate (PPF), poly acrylic acid (PAA), polyhydroxybutyric acid, hydrolysed polyacrylonitrile, polymethacrylic acid, polyethylene amine, esters of alginic acid; pectinic acid; and alginate, fully or partially oxidized alginate, hyaluronic acid, carboxy methyl cellulose, heparin, heparin sulfate, chitosan, carboxymethyl chitosan, chitin, pullulan, gellan, xanthan, collagen, gelatin, carboxymethyl starch, carboxymethyl dextran, chondroitin sulfate, cationic guar, cationic starch, and combinations thereof. In certain embodiments, the biomaterial is selected from the group consisting of alginate, fully or partially oxidized alginate, and combinations thereof.

The scaffolds of the present invention may comprise an external surface. Alternatively, or in addition, the scaffolds may comprise an internal surface. External or internal surfaces of the scaffolds of the present invention may be solid or porous. Pore size of the scaffolds can be less than about 10 nm, between about 100 nm-20 μm, or greater than about 20 μm, e.g., up to and including 1000 m in diameter. For example, the pores may be nanoporous, microporous, or macroporous. For example, the diameter of nanopores are less than about 10 nm; micropore are in the range of about 100 nm-20 m in diameter; and, macropores are greater than about 20 μm, e.g., greater than about 100 μm, e.g., greater than about 400 μm, e.g., greater than 600 m or greater than 800 μm.

In some embodiments, the scaffolds of the present invention are organized in a variety of geometric shapes (e.g., discs, beads, pellets), niches, planar layers (e.g., thin sheets). For example, discs of about 0.1-200 millimeters in diameter, e.g., 5, 10, 20, 40, 50 millimeters may be implanted subcutaneously. The disc may have a thickness of 0.1 to 10 millimeters, e.g., 1, 2, 5 millimeters. The discs are readily compressed or lyophilized for administration to a patient. An exemplary disc for subcutaneous administration has the following dimensions: 8 millimeters in diameter and 1 millimeter in thickness.

In some embodiments, the scaffolds may comprise multiple components and/or compartments. In certain embodiments, a multiple compartment device is assembled in vivo by applying sequential layers of similarly or differentially doped gel or other scaffold material to the target site. For example, the device is formed by sequentially injecting the next, inner layer into the center of the previously injected material using a needle, forming concentric spheroids. In certain embodiments, non-concentric compartments are formed by injecting material into different locations in a previously injected layer. A multi-headed injection device extrudes compartments in parallel and simultaneously. The layers are made of similar or different biomaterials differentially doped with pharmaceutical compositions. Alternatively, compartments self-organize based on their hydro-philic/phobic characteristics or on secondary interactions within each compartment. In certain embodiments, multicomponent scaffolds are optionally constructed in concentric layers each of which is characterized by different physical qualities such as the percentage of polymer, the percentage of crosslinking of polymer, chemical composition of the hydrogel, pore size, porosity, and pore architecture, stiffness, toughness, ductility, viscoelasticity, and/or composition of bioactive substances such as growth factors, homing/migration factors, differentiation factors.

In an exemplary embodiment, the device of the present disclosure comprises a polymer scaffold, a click reagent of the invention, and one or more (i.e., one or more, two or more, three or more, or four) of the following: (i) a chemoattractant; (ii) an adjuvant; (iii) an antigen; and (iv) porogen hydrogel microbeads. Additional embodiments and features of the device are described below.

### Hydrogel and Cryogel Scaffolds

In certain embodiments, the scaffolds of present invention comprises one or more hydrogels. A hydrogel is a polymer gel comprising a network of crosslinked polymer chains. A hydrogel is usually a composition comprising polymer chains that are hydrophilic. The network structure of hydrogels allows them to absorb significant amounts of water. Some hydrogels are highly stretchable and elastic; others are viscoelastic. Hydrogel are sometimes found as a colloidal gel in which water is the dispersion medium. In certain embodiments, hydrogels are highly absorbent (they can contain over 99% water) natural or synthetic polymers that possess a degree of flexibility very similar to natural tissue, due to their significant water content. In certain embodiments, a hydrogel may have a property that, when an appropriate shear stress is applied, the deformable hydrogel is dramatically and reversibly compressed (up to 95% of its volume), resulting in injectable macroporous preformed scaffolds. Hydrogels have been used for therapeutic applications, e.g., as vehicles for in vivo delivery of therapeutic agents, such as small molecules, cells and biologics. Hydrogels are commonly produced from polysaccharides, such as alginates. The polysaccharides may be chemically manipulated to modulate their properties and properties of the resulting hydrogels.

The hydrogels of the present invention are porous or non-porous. For example, the hydrogels are nanoporous having a diameter of less than about 10 nm; microporous wherein the diameter of the pores are preferably in the range of about 100 nm-20 μm; or macroporous wherein the diameter of the pores are greater than about 20 μm, more preferably greater than about 100 μm and even more preferably greater than about 400 μm. In certain embodiments, the hydrogel is macroporous with aligned pores of about 400-500 μm in diameter. Methods of preparing porous hydrogel products are known in the art. (See, e.g., U.S. Pat. No. 6,511,650, incorporated herein by reference).

The hydrogel may be constructed out of a number of different rigid, semi-rigid, flexible, gel, self-assembling, liquid crystalline, or fluid compositions such as peptide polymers, polysaccharides, synthetic polymers, hydrogel materials, ceramics (e.g., calcium phosphate or hydroxyapatite), proteins, glycoproteins, proteoglycans, metals and metal alloys. The compositions are assembled into hydrogels using methods known in the art, e.g., injection molding, lyophillization of preformed structures, printing, self-assembly, phase inversion, solvent casting, melt processing, gas foaming, fiber forming/processing, particulate leaching or a combination thereof. The assembled devices are then implanted or administered to the body of an individual to be treated.

The device comprising a hydrogel may be assembled in vivo in several ways. The hydrogel is made from a gelling material, which is introduced into the body in its ungelled form where it gels in situ. Exemplary methods of delivering device components to a site at which assembly occurs include injection through a needle or other extrusion tool, spraying, painting, or methods of deposit at a tissue site, e.g., delivery using an application device inserted through a cannula. In some embodiments, the ungelled or unformed hydrogel material is mixed with pharmaceutical compositions prior to introduction into the body or while it is introduced. The resultant in vivo/in situ assembled device, e.g., hydrogel, contains a mixture of these pharmaceutical composition(s).

In situ assembly of the hydrogel may occur as a result of spontaneous association of polymers or from synergistically or chemically catalyzed polymerization. Synergistic or chemical catalysis is initiated by a number of endogenous factors or conditions at or near the assembly site, e.g., body temperature, ions or pH in the body, or by exogenous factors or conditions supplied by the operator to the assembly site, e.g., photons, heat, electrical, sound, or other radiation directed at the ungelled material after it has been introduced. The energy is directed at the hydrogel material by a radiation beam or through a heat or light conductor, such as a wire or fiber optic cable or an ultrasonic transducer. Alternatively, a shear-thinning material, such as an ampliphile, is used which re-cross links after the shear force exerted upon it, for example by its passage through a needle, has been relieved.

In some embodiments, the hydrogel may be assembled ex vivo. In some embodiments, the hydrogel is injectable. For example, the hydrogels are created outside of the body as macroporous scaffolds. The hydrogels can be injected into the body because the pores collapse and the gel becomes very small and can fit through a needle. See, e.g., WO 12/149358; and Bencherif et al. Proc. Natl. Acad. Sci. USA 109.48(2012):19590-5, the content of which are incorporated herein by reference).

Suitable hydrogels for both in vivo and ex vivo assembly of hydrogel devices are well known in the art and described, e.g., in Lee et al., 2001, Chem. Rev. 7:1869-1879. The peptide amphiphile approach to self-assembly assembly is described, e.g., in Hartgerink et al., 2002, Proc. Natl. Acad. Sci. U.S.A 99:5133-5138. A method for reversible gellation following shear thinning is exemplified in Lee et al., 2003, Adv. Mat. 15:1828-1832.

In certain embodiments, exemplary hydrogels are comprised of materials that are compatible with encapsulation of materials including polymers, nanoparticles, polypeptides, and cells. Exemplary hydrogels are fabricated from as alginate, polyethylene glycol (PEG), PEG-acrylate, agarose, or synthetic protein (e.g., collagen or engineered proteins (i.e., self-assembly peptide-based hydrogels)). For example, a commercially available hydrogel includes BD™ PuraMatrix™. BD™ PuraMatrix™ Peptide Hydrogel is a synthetic matrix that is used to create defined three dimensional (3D) micro-environments for cell culture.

In some embodiments, the hydrogel is a biocompatible polymer matrix that is biodegradable in whole or in part. Examples of materials which can form hydrogels include alginates and alginate derivatives, polylactic acid, polyglycolic acid, poly(lactic-co-glycolic acid) (PLGA) polymers, gelatin, collagen, agarose, natural and synthetic polysaccharides, polyamino acids such as polypeptides particularly poly(lysine), polyesters such as polyhydroxybutyrate and poly-epsilon.-caprolactone, polyanhydrides; polyphosphazines, poly(vinyl alcohols), poly(alkylene oxides) particularly poly(ethylene oxides), poly(allylamines)(PAM), poly(acrylates), modified styrene polymers such as poly(4-aminomethylstyrene), pluronic polyols, polyoxamers, poly(uronic acids), poly(vinylpyrrolidone), and copolymers of the above, including graft copolymers. Synthetic polymers and naturally-occurring polymers such as, but not limited to, collagen, fibrin, hyaluronic acid, agarose, and laminin-rich gels may also be used.

The implantable device can have virtually any regular or irregular shape including, but not limited to, spheroid, cubic, polyhedron, prism, cylinder, rod, disc, or other geometric shape. Accordingly, in some embodiments, the implant is of cylindrical form from about 0.5 to about 10 mm in diameter and from about 0.5 to about 10 cm in length. Preferably, its diameter is from about 1 to about 5 mm and length from about 1 to about 5 cm.

In some embodiments, the devices of the invention are of spherical form. When the implantable device is in a spherical form, its diameter can range, in some embodiments, from about 0.5 to about 50 mm in diameter. In some embodiments, a spherical implant's diameter is from about 5 to about 30 mm. In an exemplary embodiment, the diameter is from about 10 to about 25 mm.

In certain embodiments, the scaffold comprises click-hydrogels and/or click-cryogels. A click hydrogel or cryogel is a gel in which cross-linking between hydrogel or cryogel polymers is facilitated by click reactions between the polymers. Each polymer may contain one of more functional groups useful in a click reaction. Given the high level of specificity of the functional group pairs in a click reaction, active compounds can be added to the preformed device prior to or contemporaneously with formation of the hydrogel device by click chemistry. Non-limiting examples of click reactions that may be used to form click-hydrogels include Copper I catalyzed azide-alkyne cycloaddition, strain-promoted assize-alkyne cycloaddition, thiol-ene photocoupling, Diels-Alder reactions, inverse electron demand Diels-Alder reactions, tetrazole-alkene photo-click reactions, oxime reactions, thiol-Michael addition, and aldehyde-hydrazide coupling. Non-limiting aspects of click hydrogels are described in Jiang et al. (2014) Biomaterials, 35:4969-4985, the entire content of which is incorporated herein by reference. Preferably, the click reagent of the present invention for metabolic labeling of cells infiltrating the scaffold is not reactive with the click hydrogel or cryogel.

In various embodiments, a click alginate is utilized (see, e.g., PCT International Patent Application Publication No. WO 2015/154078 published Oct. 8, 2015, hereby incorporated by reference in its entirety).

Exemplary click-hydrogel devices and scaffold materials include a hydrogel comprising a first polymer and a second polymer, where the first polymer is connected to the second polymer by linkers of formula (A):

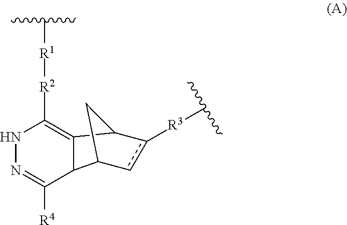

wherein
   bond [symbol] is single or a double bond:
R¹ is —C₀-C₆alkyl-NR^{2N}—, —C₀-C₆alkyl-O—, or —C₀-C₃alkyl-C(O)—;
R² is a bond, aryl, or heteroaryl, wherein aryl and heteroaryl are optionally substituted with halogen, hydroxy, C₁-C₆alkyl, C₁-C₆alkoxy, (C₁-C₆alkyl)amino, or di(C₁-C₆alkyl)amino;
R³ is —C₀-C₆alkyl-NR^{2N}—, —C₀-C₆alkyl-O—, or —C₀-C₃alkyl-C(O)—; and R⁴ is hydrogen, C₁-C₆alkyl, aryl, or heteroaryl, wherein aryl and heteroaryl are optionally substituted with halogen, hydroxy, C₁-C₆alkyl, C₁-C₆alkoxy, (C₁-C₆alkyl)amino, or di(C₁-C₆alkyl)amino.
R^{2N} is independently hydrogen, C₁-C₆ alkyl, aryl, heteroaryl, R²N, or R², wherein C¹-C⁶ alkyl, aryl and heteroaryl are optionally substituted with halogen, hydroxy, C₁-C₆ alkyl, C₁-C₆ alkoxy, (C₁-C₆ alkyl)amino, or di(C₁-C₆ alkyl)amino. In one embodiment, the hydrogel of the disclosure is wherein the linkers of formula (A) are of the form of formula (I):

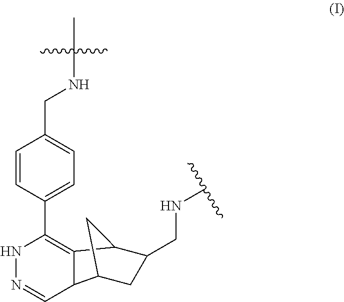 [SMILES: *NCc1ccc(C2=C3C(C=NN2)C2CC(CN*)C3C2)cc1]

or by formula (II):

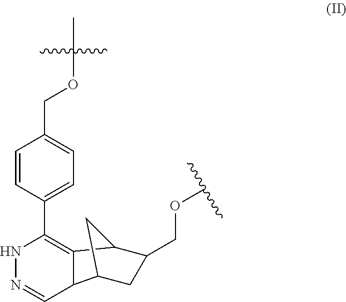 [SMILES: *OCc1ccc(C2=C3C(C=NN2)C2CC(CO*)C3C2)cc1]

or by formula (III):

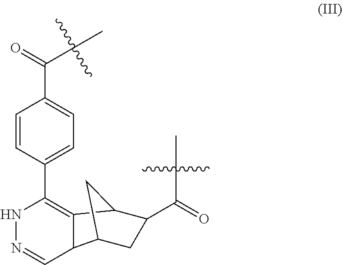 [SMILES: *C(=O)c1ccc(C2=C3C(C=NN2)C2CC(C(*)=O)C3C2)cc1]

wherein the linkers of formula (I), (II), or (III) are optionally substituted at any suitable position.

Another embodiment provides the linkers of formula (A) according to any preceding embodiment, wherein R¹ is:
a. —NR^{2N}—, —C₁-C₆ alkyl-NR^{2N}—, —C₁-C₆alkyl-O—, —C(O)—, or —C₁-C₃alkyl-C(O)—;
b. —C₀-C₆alkyl-NR^{2N}—;
c. —C₁-C₆ alkyl-NR^{2N}N—;
d. —C₁-C₃ alkyl-NR^{2N}N—;
e. -methyl-NH— or -pentyl-NH—;
f. —C₀-C₆ alkyl-O—;
g. —C₁-C₆ alkyl-O—;
h. —C₁-C₃ alkyl-O—;
i. -methyl-O— or -pentyl-O—;
j. —C₀-C₃ alkyl-C(O)—;
k. —C(O)—;
l. -methyl-C(O)—;
m. the same as R³.

NR²N is independently hydrogen, C₁-C₆ alkyl, aryl, heteroaryl, R²N, or R², wherein C₁-C₆ alkyl, aryl and heteroaryl are optionally substituted with halogen, hydroxy, C₁-C₆ alkyl, C₁-C₆ alkoxy, (C₁-C₆ alkyl)amino, or di(C₁-C₆ alkyl)amino.

Another embodiment provides the linkers of formula (A) according to any preceding embodiment, wherein R² is a bond.

In one embodiment, the linkers of formula (A) according to any preceding embodiment are those wherein R² is
a. aryl or heteroaryl, each optionally substituted;
b. optionally substituted aryl;
c. phenyl;
d. optionally substituted heteroaryl; or
e. pyridyl, pyrimidyl, or pyrazinyl.

Another embodiment provides the linkers of formula (A) according to any preceding embodiment, wherein R³ is
a. —NR^{2N}—, —C₁-C₆ alkyl-NR2N—, —O—, —C₁-C₆ alkyl-O—, —C(O)—, or —C₁-C₃alkyl-C(O)—;
b. —C₀-C₆ alkyl-NR^{2N}N—;
c. —C₁-C₆ alkyl-NR^{2N}N—;
d. —C₁-C₃ alkyl-NR^{2N}N—;
e. -methyl-NH— or -pentyl-NH—;
f. —C₀-C₆ alkyl-O—;
g. —C₁-C₆ alkyl-O—;
h. —C₁-C₃ alkyl-O—;
i. -methyl-O— or -pentyl-O—;
j. —C₀-C₃ alkyl-C(O)—;
k. —C(O)—;
l. -methyl-C(O)—; or
m. the same as R¹.

R^{2N} is independently hydrogen, C₁-C₆ alkyl, aryl, heteroaryl, R²N, or R², wherein C₁-C₆ alkyl, aryl and heteroaryl are optionally substituted with halogen, hydroxy, C₁-C₆ alkyl, C₁-C₆ alkoxy, (C₁-C₆ alkyl)amino, or di(C₁-C₆ alkyl)amino. In one embodiment, the linkers of formula (A) according to any preceding embodiment are those wherein R⁴ is hydrogen.

In one embodiment, the linkers of formula (A) according to any preceding embodiment are those wherein R⁴ is
a. C₁-C₆alkyl, aryl, or heteroaryl, wherein aryl and heteroaryl are optionally substituted;
b. aryl or heteroaryl, wherein aryl and heteroaryl are optionally substituted;
c. optionally substituted aryl;
d. phenyl;
e. optionally substituted heteroaryl; or
f. pyridyl, pyrimidyl, or pyrazinyl.

Another embodiment provides the linkers of formula (A) according to any preceding embodiment, wherein R⁴ is C₁-C₆ alkyl, C₁-C₃ alkyl, or methyl.

In some embodiments, the hydrogel comprises a plurality of linkers of formula (A); or formula (I), formula (II), or formula (III).

The invention also includes a hydrogel comprising an interconnected network of a plurality of polymers, e.g., including a first polymer and a second polymer. For example, the polymers are connected via a plurality of linkers of formula (A), or of formula (I), formula (II), or formula (III).

Some embodiments of the disclosure provide hydrogels wherein the first polymer and the second polymer are independently soluble polymers. In other embodiments, the first polymer and the second polymer are independently water-soluble polymers.

In some embodiments, the concentration of crosslinks per hydrogel (e.g., where each crosslink comprises formula I) is at least about 10% (w/w), e.g., at least about 10%, about 15%, about 20%, about 30%, about 40%, about 50%, about 60%, about 70%, about 80%, about 90%, about 95%, about 97%, about 99%, or about 100% (w/w).

The first polymer and the second polymer can be the same or different. In some embodiments, the first polymer and the second polymer are the same type of polymer. In other embodiments, the first polymer and/or the second polymer comprise a polysaccharide. For example, the first polymer and the second polymer can both comprise a polysaccharide. In some embodiments, the first polymer and/or the second polymer are independently selected from the group consisting of alginate, chitosan, polyethylene glycol (PEG), gelatin, hyaluronic acid, collagen, chondroitin, agarose, polyacrylamide, and heparin. In some embodiments, the first polymer and the second polymer are the same polymer independently selected from the group consisting of alginate, chitosan, polyethylene glycol (PEG), gelatin, hyaluronic acid, collagen, chondroitin, agarose, polyacrylamide, and heparin.

Such scaffolds and scaffold materials, as well as methods for producing such scaffolds, are described in PCT International Patent Application Publication No. WO 2015/154078 published Oct. 8, 2015, the entire content of which is incorporated herein by reference. For example, a click hydrogel may be prepared in a process: a) providing a first polymer comprising a first click reaction moiety and a second polymer comprising a second click reaction moiety. In non-limiting examples, the first click reaction moiety and the second click reaction moiety may be react with each other in a copper I catalyzed azide-alkyne cycloaddition, strain-promoted assize-alkyne cycloaddition, thiol-ene photo coupling, a Diels-Alder reaction, an inverse electron demand Diels-Alder reaction, a tetrazole-alkene photo-click reaction, a oxime reaction, a thiol-Michael addition, or via aldehyde-hydrazide coupling. In an embodiment, the first click reaction moiety is a diene moiety and the second click reaction moiety is a dienophile moiety. In an embodiment, the first click reaction moiety is a tetrazine moiety and the second click reaction moiety is a norbornene moiety. As used herein, the terms “tetrazine” and “tetrazine moiety” include molecules that comprise 1,2,4,5-tetrazine substituted with suitable spacer for linking to the polymer (e.g., alkylamines like methylamine or pentylamine), and optionally further substituted with one or more substituents at any available position. Exemplary tetrazine moieties suitable for the compositions and methods of the disclosure are described in Karver et al. Bioconjugate Chem. 22(2011):2263-2270, and WO 2014/065860, both incorporated herein by reference). As used herein, the terms “norbornene” and “norbornene moieties” include but are not limited to norbornadiene and norbornene groups further comprising suitable spacer for linking to the polymer (e.g., alkylamines like methylamine or pentylamine), and optionally further substituted with one or more substituents at any available position. Such moieties include, for example, norbornene-S-methylamine and norbomadienemethylamine.

Accordingly, some embodiments feature a cell-compatible and optionally, cell-adhesive, highly crosslinked hydrogel (e.g., cryogel) polymer composition comprising open interconnected pores, wherein the hydrogel (e.g., cryogel) is characterized by shape memory following deformation by compression or dehydration. The device has a high density of open interconnected pores. Also, the hydrogel (e.g., cryogel) comprises a crosslinked gelatin polymer or a crosslinked alginate polymer.

In certain embodiments, a hydrogel (e.g., cryogel) system can deliver one or more agent (e.g., a chemoattractant such as GM-CSF, and/or an adjuvant, such as a specific TLR agonist (such as CpG-ODN), while creating a space for cells (e.g., immune cells such as dendritic cells (DCs)) infiltration and trafficking. In some embodiments, the hydrogel system according the present invention deliver GM-CSF that acts as a DC enhancement/recruitment factor, and CpG ODN as an adjuvant that is a specific TLR agonist (DC activation factor).

In some embodiment, cryogel devices, such as MA-alginate, can function as a labeling platform by creating a local niche, such as immunogenic niche. In some embodiments, the cryogel creates a local niche in which the encounter of cells, such as immune cells, and various exemplary agent of the invention, such as the click functionalized polysaccharide polymer can be controlled. In certain embodiments, the cells and the exemplary agents of the present invention are localized into a small volume, and the labeling of the cells can be quantitatively controlled in space and time.

In non-limiting example, the hydrogel (e.g., cryogel) can be engineered to coordinate the delivery of both adjuvant and antigen in space and time, potentially enhancing overall anti-tumor performance by adjusting the activation and/or maturing of recruited immune cells, such as DCs. In certain embodiments, the cells and immunomodulatory agents are localized into a small volume, and the delivery of factors in space and time can be quantitatively controlled. As the immunomodulatory factors are released locally, few systemic effects are anticipated, in contrast to systemically delivered agents, such as immune checkpoint blocking antibodies.

Examples of polymer compositions from which the cryogel or hydrogel is fabricated are described throughout the present disclosure, and include alginate, hyaluronic acid, gelatin, heparin, dextran, carob gum, PEG, PEG derivatives including PEG-co-PGA and PEG-peptide conjugates. The techniques can be applied to any biocompatible polymers, e.g. collagen, chitosan, carboxymethylcellulose, pullulan, polyvinyl alcohol (PV A), Poly(2-hydroxyethyl methacrylate) (PHEMA), Poly(N-isopropylacrylamide) (PNIPAAm), or Poly(acrylic acid) (PAAc). For example, the composition comprises an alginate-based hydrogel/cryogel. In another example, the scaffold comprises a gelatin-based hydrogel/cryogel.

Cryogels are a class of materials with a highly porous interconnected structure that are produced using a cryotropic gelation (or cryogelation) technique. Cryogels also have a highly porous structure. Typically, active compounds are added to the cryogel device after the freeze formation of the pore/wall structure of the cryogel. Cryogels are characterized by high porosity, e.g., at least about 50, 55, 60, 65, 70, 75, 80, 85, 90, or 95% pores with thin pore walls that are characterized by high density of polymer crosslinking. The walls of cryogels are typically dense and highly cross-linked, enabling them to be compressed through a needle into a subject without permanent deformation or substantial structural damage.

In various embodiments, the pore walls comprise at least about 10, 15, 20, 25, 30, 35, 40, 10-40% or more polymer. In some embodiments, a polymer concentration of about 0.5-4% (before the cryogelation) is used, and the concentration increases substantially by the completion of cryogelation. Non-limiting aspects of cryogel gelation and the increase of polymer concentration after cryogelation are discussed in Beduer et al. (2015) Advanced Healthcare Materials Volume 4, Issue 2, pages 301-312, the entire content of which is incorporated herein by reference.

In certain embodiments, cryogelation comprises a technique in which polymerization-crosslinking reactions are conducted in quasi-frozen reaction solution. Non-limiting examples of cryogelation techniques are described in U.S. Patent Application Publication No. 201410227327, published Aug. 14, 2014, the entire content of which is incorporated herein by reference. An advantage of cryogels compared to conventional macroporous hydrogels obtained by phase separation is their high reversible deformability. Cryogels may be extremely soft but can be deformed and reform their shape. In certain embodiments, cyrogels can be very tough, and can withstand high levels of deformations, such as elongation and torsion and can also be squeezed under mechanical force to drain out their solvent content. In certain embodiments, improved deformability properties of alginate cryogels originate from the high crosslinking density of the unfrozen liquid channels of the reaction system.

In some embodiments, the invention also features gelatin scaffolds, e.g., gelatinhydrogels such as gelatin cryogels, which are a cell-responsive platform for biomaterial-based therapy. Gelatin is a mixture of polypeptides that is derived from collagen by partial hydrolysis. These gelatin scaffolds have distinct advantages over other types of scaffolds and hydrogels/cryogels. For example, the gelatin scaffolds of the invention support attachment, proliferation, and survival of cells and are degraded by cells, e.g., by the action of enzymes such as matrix metalloproteinases (MMPs) (e.g., recombinant matrix metalloproteinase-2 and -9).

In certain embodiments, prefabricated gelatin cryogels rapidly reassume their original shape (“shape memory”) when injected subcutaneously into a subject (e.g., a mammal such as a human, dog, cat, pig, or horse) and elicit little or no harmful host immune response (e.g., immune rejection) following injection.

In some embodiments, the hydrogel (e.g., cryogel) comprises polymers that are modified, e.g., sites on the polymer molecule are modified with a methacrylic acid group (methacrylate (MA)) or an acrylic acid group (acrylate). Exemplary modified hydrogels/cryogels are MA-alginate (methacrylated alginate) or MA-gelatin. In the case of MA-alginate or MA-gelatin, 50% corresponds to the degree of methacrylation of alginate or gelatin. This means that every other repeat unit contains a methacrylated group. The degree of methacrylation can be varied from about 1% to about 100%. Preferably, the degree of methacrylation varies from about 1% to about 90%.

In certain embodiments, polymers can also be modified with acrylated groups instead of methacrylated groups. The product would then be referred to as an acrylated-polymer. The degree of methacrylation (or acrylation) can be varied for most polymers. However, some polymers (e.g. PEG) maintain their water-solubility properties even at 100% chemical modification. After crosslinking, polymers normally reach near complete methacrylate group conversion indicating approximately 100% of cross-linking efficiency. For example, the polymers in the hydrogel are 50-100% crosslinked (covalent bonds). The extent of crosslinking correlates with the durability of the hydrogel. Thus, a high level of crosslinking (90-100%) of the modified polymers is desirable.

For example, the highly crosslinked hydrogel/cryogel polymer composition is characterized by at least about 50% polymer crosslinking (e.g., about 75%, 80%, 85%, 90%, 95%, 98%, 99%, 100%). The high level of crosslinking confers mechanical robustness to the structure. Preferably, the percentage of crosslinking is less than about 100%. The composition is formed using a free radical polymerization process and a cryogelation process.

For example, the cryogel is formed by cryopolymerization of methacrylated gelatin or methacrylated alginate. In some embodiments, the cryogel comprises a methacrylated gelatin macro monomer or a methacrylated alginate macromonomer at concentration of about 1.5% (w/v) or less (e.g., about, 1.5%, 1.4%, 1.3%, 1.2%, 1.1%, 1%, 0.9%, 0.8%, 0.7%, 0.6%, 0.5%, 0.4%, 0.3%, 0.2% or less). In some embodiments, the methacrylated gelatin or alginate macromonomer concentration is about 1% (w/v).

In certain embodiments, the cryogel comprises at least about 75% pores, e.g., about 80%, 85%, 90%, 91%, 92%, 93%, 94%, 95%, 96%, 97%, 98%, or 99% or more pores. In some embodiments, the pores are interconnected. Interconnectivity is important to the function of the hydrogel and/or cryogel, as without interconnectivity, water would become trapped within the gel. Interconnectivity of the pores permits passage of water (and other compositions such as cells and compounds) in and out of the structure. In certain embodiments, in a fully hydrated state, the hydrogel (e.g., cryogel) comprises at least about 90% water (e.g., between about 90-99%, at least about 92%, 95%, 97%, 99%, or more) water. For example, at least about 90% (e.g., at least about 92%, 95%, 97%, 99%, or more) of the volume of the cryogel is made of liquid (e.g., water) contained in the pores. In certain embodiments, in a compressed or dehydrated hydrogel, up to about 50%, 60%, 70% of that water is absent, e.g., the cryogel comprises less than about 25% (e.g., about 20%, 15%, 10%, 5%, or less) water.

In certain embodiments, the cryogels of the invention comprises pores large enough for a cell to travel through. For example, the cryogel contains pores of about 20-500 μm in diameter, e.g., about 20-30 μm, about 30-150 μm, about 50-500 μm, about 50-450 μm, about 100-400 μm, about 200-500 μm. In some embodiments, the hydrated pore size is about 1-500 μm (e.g., about 10-400 μm, about 20-300 μm, about 50-250 μm).

In some embodiments, injectable hydrogels or cryogels are further functionalized by addition of a functional group chosen from the group consisting of: amino, vinyl, aldehyde, thiol, silane, carboxyl, azide, alkyne. Alternatively or in addition, the cryogel is further functionalized by the addition of a further cross-linker agent (e.g. multiple arms polymers, salts, aldehydes, etc.). The solvent can be aqueous, and in particular acidic or alkaline. The aqueous solvent can comprise a water-miscible solvent (e.g. methanol, ethanol, DMF, DMSO, acetone, dioxane, etc).

For cryogels, the cryo-crosslinking may take place in a mold and the cryogels (which may be injected) can be degradable. The pore size can be controlled by the selection of the main solvent used, the incorporation of a porogen, the freezing temperature and rate applied, the crosslinking conditions (e.g. polymer concentration), and also the type and molecule weight of the polymer used. The shape of the cryogel may be dictated by a mold and can thus take on any shape desired by the fabricator, e.g., various sizes and shapes (disc, cylinders, squares, strings, etc.) are prepared by cryogenic polymerization.

Injectable cryogels can be prepared in the micrometer-scale to millimeter-scale. Exemplary volumes vary from a few hundred μm³ (e.g., about 100-500 μm³)) to over 100 mm³. In certain embodiment, an exemplary scaffold composition is between about 100 μm³ to 100 mm³ in size (e.g., between about 1 mm³ and 10 mm³ in size).

In some embodiments, the cryogels are hydrated, loaded with compounds and loaded into a syringe or other delivery apparatus. For example, the syringes are prefilled and refrigerated until use. In another example, the cryogel is dehydrated, e.g., lyophylized, optionally with a compound (such as a chemoattractant) loaded in the gel and stored dry or refrigerated. Prior to administration, a cryogel-loaded syringe or apparatus may be contacted with a solution containing compounds to be delivered. For example, the barrel of the cryogel pre-loaded syringe is filled with a physiologically-compatible solution, e.g., phosphate-buffered saline (PBS). In some embodiments, the cryogel may be administered to a desired anatomical site followed by the volume of solution, optionally containing other ingredients, e.g., a chemoattractant alone or together with one or more compounds disclosed herein. The cryogel is then rehydrated and regains its shape integrity in situ. In certain embodiments, the volume of PBS or other physiologic solution administered following cryogel placement is generally about 10 times the volume of the cryogel itself.

The cryogel also has the advantage that, upon compression, the cryogel composition maintains structural integrity and shape memory properties. For example, the cryogel is injectable through a hollow needle. For example, the cryogel returns to its original geometry after traveling through a needle (e.g., a 16 gauge (G) needle, e.g., having a 1.65 mm inner diameter). Other exemplary needle sizes are 16-gauge, an IS-gauge, a 20-gauge, a 22-gauge, a 24-gauge, a 26-gauge, a 2S-gauge, a 30-gauge, a 32-gauge, or a 34-gauge needle. Injectable cryogels have been designed to pass through a hollow structure, e.g., very fine needles, such as 18-30 G needles.

The injectable cryogels may be molded to a desired shape, in the form of rods, square, disc, spheres, cubes, fibers, foams. In some cases, the cryogel comprises the shape of a disc, cylinder, square, rectangle, or string. For example, the cryogel composition is between about 100 μm³ to 100 mm³ in size, e.g., between 1 mm³ to 50 mm³ in size. For example, the cryogel composition is between about 1 mm in diameter to about 50 mm in diameter (e.g., about 5 mm). Optionally, the thickness of the cryogel is between about 0.2 mm to about 50 mm (e.g., about 2 mm).

In some examples, the scaffold composition comprises a cell adhesion composition chemically linked, e.g., covalently attached, to a polymer. For example, the cell adhesion composition comprises a peptide comprising an RGD amino acid sequence. In non-limiting examples, the hydrogel or cryogel composition (e.g., gelatin) has cell-adhesive properties. In some embodiments, the scaffold composition is not modified with a cell adhesive molecule, such as arginine-glycine-aspartate (RGD).

Three exemplary cryogel materials systems are described below.
a) Methacrylated gelatin cryogel (CryoGeIMA)—An exemplary cryogel utilized methacrylated gelatin and the results are described in detail in U.S. Patent Application Publication No. 2014-0227327, published Aug. 14, 2014, the entire contents of which are incorporated herein by reference.
b) Methacrylated alginate cryogel (CryoMAAlginate)—An exemplary cryogel utilized methacrylated alginate and the results are described in detail in U.S. Patent Application Publication No. 2014-0227327, published Aug. 14, 2014, the entire contents of which are incorporated herein by reference.
c) Click Alginate cryogel with Laponite nanoplatelets (CryoClick)—The base material is click alginate (PCT Intemational Patent Application Publication No. WO 2015/154078 published Oct. 8, 2015, hereby incorporated by reference in its entirety). In some examples, the base material contains laponite (commercially available silicate clay used in many consumer products such as cosmetics). Laponite has a large surface area and highly negative charge density which allows it to adsorb positively charged moieties on a variety of proteins and other biologically active molecules by an electrostatic interaction, allowing drug loading. When placed in an environment with a low concentration of drug, adsorbed drug releases from the laponite in a sustained manner. This system allows release of a more flexible array of immunomodulators compared to the base material alone.

Various embodiments of the present subject matter include delivery vehicles comprising a pore-forming scaffold composition. For example, pores (such as macropores) are formed in situ within a hydrogel following hydrogel injection into a subject. Pores that are formed in situ via degradation of a sacrificial porogen hydrogel within the surrounding hydrogel (bulk hydrogel) facilitate recruitment and trafficking of cells, as well as the release of any composition or agent of the present invention, for example, compounds, such as an immunostimulatory compound; a compound that attracts an immune cell to or into the delivery vehicle, or an antigen, or any combination thereof. In some embodiments, the sacrificial porogen hydrogel, the bulk hydrogel, or both the sacrificial porogen hydrogel and the bulk hydrogel may comprise any composition or agent of the present invention, for example, an immunostimulatory compound, a compound that attracts an immune cell to or into the delivery vehicle, a compound that inhibits an immuneinhibitory protein, and/or an antigen, or any combination thereof.

In various embodiments, the pore-forming composition becomes macroporous over time when resident in the body of a recipient animal such as a mammalian subject. For example, the pore-forming composition may comprise a sacrificial porogen hydrogel and a bulk hydrogel, wherein the sacrificial porogen hydrogel degrades at least about 10% faster (e.g., at least about 15%, at least about 20%, at least about 25%, at least about 30%, at least about 35%, at least about 40%, at least about 45%, or at least about 50% faster) than the bulk hydrogel. The sacrificial porogen hydrogel may degrade leaving macropores in its place. In certain embodiments, the macropores are open interconnected macropores. In some embodiments, the sacrificial porogen hydrogel may degrade more rapidly than the bulk hydrogel, because the sacrificial porogen hydrogel (i) is more soluble in water (comprises a lower solubility index), (ii) is cross-linked to protease-mediated degradation motifs as described in U.S. Patent Application Publication No. 2005-0119762, published Jun. 2, 2005 (incorporated herein by reference), (iii) comprises a shorter polymer that degrades more quickly compared to that of a longer bulk hydrogel polymer, (iv) is modified to render it more hydrolytically degradable than the bulk hydrogel (e.g., by oxidation), and/or (v) is more enzymatically degradable compared to the bulk hydrogel.

In various embodiments, a device or scaffold is loaded (e.g., soaked with) with one or more active compounds after polymerization. In certain embodiments, device or scaffold polymer forming material is mixed with one or more active compounds before polymerization. In some embodiments, a device or scaffold polymer forming material is mixed with one or more active compounds before polymerization, and then is loaded with more of the same or one or more additional active compounds after polymerization.

In some embodiments, pore size or total pore volume of a device or scaffold is selected to influence the release of compounds from the device or scaffold. Exemplary porosities (e.g., nanoporous, microporous, and macroporous scaffolds and devices) and total pore volumes (e.g., about 5, 10, 15, 20, 25, 30, 35, 40, 45, 50, 55, 60, 65, 70, 75, 80, 85, 90, 95% or more) are described herein. Increased pore size and total pore volume increases the amount of compounds that can be delivered into or near a tissue, such as tumor tissue. In some embodiments, a pore size or total pore volume is selected to increase the speed at which active ingredients exit the device or scaffold. In various embodiments, an active ingredient may be incorporated into the scaffold material of a hydrogel or cryogel, e.g., to achieve continuous release of the active ingredient from the scaffold or device over a longer period of time compared to active ingredient that may diffuse from a pore cavity.

Porosity influences recruitment the cells into devices and scaffolds and the release of substances from devices and scaffolds. Pores may be, e.g., nanoporous, microporous, or macroporous. For example, the diameter of nanopores is less than about 10 nm. Micropores are in the range of about 100 nm to about 20 μm in diameter. Macropores are greater than about 20 μm (e.g., greater than about 100 μm or greater than about 400 μm). Exemplary macropore sizes include about 50 μm, about 100 μm, about 150 μm, about 200 μm, about 250 μm, about 300 μm, about 350 μm, about 400 μm, about 450 μm, about 500 μm, about 550 μm, and about 600 μm. Macropores are those of a size that permit a eukaryotic cell to traverse into or out of the composition. In one example, a macroporous composition has pores of about 400 μm to about 500 μm in diameter. The preferred pore size depends on the application.

In various embodiments, the device is manufactured in one stage in which one layer or compartment is made and infused or coated with one or more compounds. Exemplary bioactive compositions comprise polypeptides or polynucleotides. In certain embodiments, the device is manufactured in two or more (3, 4, 5, 6, . . . 10 or more) stages in which one layer or compartment is made and infused or coated with one or more compounds followed by the construction of a second, third, fourth or more layers, which are in turn infused or coated with one or more compounds in sequence. In some embodiments, each layer or compartment is identical to the others or distinguished from one another by the number or mixture of bioactive compositions as well as distinct chemical, physical and biological properties. Polymers that may be formulated for specific applications by controlling the molecular weight, rate of degradation, and method of scaffold formation. Coupling reactions can be used to covalently attach bioactive epitopes, such as the cell adhesion sequence RGD to the polymer backbone.

In some embodiments, one or more compounds is added to the scaffold compositions using a known method including surface absorption, physical immobilization, e.g., using a phase change to entrap the substance in the scaffold material. For example, an immunostimulatory compound is mixed with the scaffold composition while it is in an aqueous or liquid phase, and after a change in environmental conditions (e.g., pH, temperature, ion concentration), the liquid gels or solidifies thereby entrapping the bioactive substance. In some embodiments, covalent coupling, e.g., using alkylating or acylating agents, is used to provide a stable, long term presentation of a compound on the scaffold in a defined conformation. Exemplary reagents for covalent coupling of such substances are provided in the table below.

TABLE 1
Methods to Covalently Couple Peptides/Proteins to Polymers

| Functional | | Reacting |
|---|---|---|
| Group of | | Groups on |
| Polymer | Coupling Reagents and Cross-Liner | Proteins/Peptides |
| —OH | Cyanogen bromide (CNBr) | —NH₂ |
| | Cyanuric chloride | |
| | 4-( 4,6-Dimethoxy-1 ,3,5- | |
| | triazin-2-yl)-4- | |
| | methylmorpholinium | |
| | chloride (DMT-MM) | |
| —NH₂ | Diisocyanate compounds | —NH₂ |
| | Diisothoncyanate compounds | —OH |
| | Glutaraldehyde Succinic anhydride | |
| —NH₂ | Nitrous Acid | —NH₂ |
| | Hydrazine + nitrous acid | —SH |
| | | —Ph—OH |
| —NH₂ | Carbodiimide compounds | —COOH |
| | (e.g., EDC, DCC)[a] | |
| | DMT-MM | |
| —COOH | Thiony I chloride | —NH₂ |
| | N-hydroxysuccinimide | |
| | N-hydroxysulfosuccinimide + EDC | |
| —SH | Disulfide compound | —SH |

[a] EDC: 1-ethyl-3-(3-dimethylaminopropyl) carbodiimide hydrochloride; DCC: dicyclohexy Icarbodiimide

### Alginate Scaffolds

In certain embodiments, the device of the invention comprises an alginate hydrogel. Alginates are versatile polysaccharide based polymers that may be formulated for specific applications by controlling the molecular weight, rate of degradation and method of scaffold formation. Alginate polymers are comprised of two different monomeric units, (1-4)-linked β-D-mannuronic acid (M units) and α L-guluronic acid (G units) monomers, which can vary in proportion and sequential distribution along the polymer chain. Alginate polymers are polyelectrolyte systems which have a strong affinity for divalent cations (e.g., Ca⁺², Mg⁺², Ba⁺²) and form stable hydrogels when exposed to these molecules. See Martinsen A., et al., Biotech. & Bioeng., 33 (1989) 79-89). For example, calcium cross-linked alginate hydrogels are useful for dental applications, wound dressings chondrocyte transplantation and as a matrix for other cell types. Without wishing to be bound by theory, it is believed that G units are preferentially crosslinked using calcium crosslinking, whereas click reaction based crosslinking is more indiscriminate with respect to G units or M units (i.e., both G and M units can be crosslinked by click chemistry). Alginate scaffolds and the methods for making them are known in the art. See, e.g., International Patent Application Publication No. WO2017/075055 A1, published on May 4, 2017, the entire contents of which are incorporated herein by reference.

The alginate polymers useful in the context of the present invention can have an average molecular weight from about 20 kDa to about 500 kDa, e.g., from about 20 kDa to about 40 kDa, from about 30 kDa to about 70 kDa, from about 50 kDa to about 150 kDa, from about 130 kDa to about 300 kDa, from about 230 kDa to about 400 kDa, from about 300 kDa to about 450 kDa, or from about 320 kDa to about 500 kDa. In one example, the alginate polymers useful in the present invention may have an average molecular weight of about 32 kDa. In another example, the alginate polymers useful in the present invention may have an average molecular weight of about 265 kDa. In some embodiments, the alginate polymer has a molecular weight of less than about 1000 kDa, e.g., less than about 900 Kda, less than about 800 kDa, less than about 700 kDa, less than about 600 kDa, less than about 500 kDa, less than about 400 kDa, less than about 300 kDa, less than about 200 kDa, less than about 100 kDa, less than about 50 kDa, less than about 40 kDa, less than about 30 kDa or less than about 25 kDa. In some embodiments, the alginate polymer has a molecular weight of about 1000 kDa, e.g., about 900 Kda, about 800 kDa, about 700 kDa, about 600 kDa, about 500 kDa, about 400 kDa, about 300 kDa, about 200 kDa, about 100 kDa, about 50 kDa, about 40 kDa, about 30 kDa or about 25 kDa. In one embodiment, the molecular weight of the alginate polymers is about 20 kDa.

Coupling reactions can be used to covalently attach bioactive agent, such as an atom, a chemical group, a nucleoside, a nucleotide, a nucleobase, a sugar, a nucleic acid, an amino acid, a peptide, a polypeptide, a protein, a protein complex, to the polymer backbone.

The term “alginate”, used interchangeably with the term “alginate polymers”, includes unmodified alginate or modified alginate. Modified alginate includes, but not limited to, oxidized alginate (e.g., comprising one or more algoxalate monomer units) and/or reduced alginate (e.g., comprising one or more algoxinol monomer units). In some embodiments, oxidized alginate comprises alginate comprising one or more aldehyde groups, or alginate comprising one or more carboxylate groups. In other embodiments, oxidized alginate comprises highly oxidized alginate, e.g., comprising one or more algoxalate units. Oxidized alginate may also comprise a relatively small number of aldehyde groups (e.g., less than 15%, e.g., 14,%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, 0.2% 0.1% or less aldehyde groups or oxidation on a molar basis). The term “alginate” or “alginate polymers” may also include alginate, e.g., unmodified alginate, oxidized alginate or reduced alginate.

### Porous and Pore-Forming Scaffolds

The scaffolds of the present invention may be nonporous or porous. In certain embodiments, the scaffolds of the present invention are porous. Porosity of the scaffold composition influences migration of the cells through the device. Pores may be nanoporous, microporous, or macroporous. For example, the diameter of nanopores is less than about 10 nm. Micropores are in the range of about 100 nm to about 20 μm in diameter. Macropores are greater than about 20 μm (e.g., greater than about 100 μm or greater than about 400 μm). Exemplary macropore sizes include about 50 μm, 100 μm, 150 μm, 200 μm, 250 μm, 300 μm, 350 μm, 400 μm, 450 μm, 500 μm, 550 μm, and 600 μm. Macropores are those of a size that permit a eukaryotic cell to traverse into or out of the composition. In certain embodiments, a macroporous composition has pores of about 400 μm to 500 μm in diameter. The size of pores may be adjusted for different purpose. For example, for cell deployment and cell release, the pore diameter may be greater than 50 μm.

In some embodiments, the scaffolds contains pores before the administration into a subject. In some embodiments, the scaffolds comprises pore-forming scaffold composition. Pore-forming scaffolds and the methods for making pore-forming scaffolds are known in the art. See, e.g., U.S. Patent Publication US2014/0079752A1, the content of which is incorporated herein by reference. In certain embodiments, the pore-forming scaffolds is not initially porous, but which becomes macroporous over time resident in the body of a recipient animal such as a mammalian subject. In certain embodiments, the pore-forming scaffolds are hydrogel scaffolds. The pore may be formed at different time, e.g., after about 12 hours, or 1, 3, 5, 7, or 10 days or more after administration, i.e. resident in the body of the subject.

In certain embodiments, the pore-forming scaffolds comprise a first hydrogel and a second hydrogel, wherein the first hydrogel degrades at least about 10% faster (e.g., at least about 15%, at least about 20%, at least about 25%, at least about 30%, at least about 35%, at least about 40%, at least about 45%, at least about 50% faster, at least about 2 times faster, or at least about 5 times faster) than the second hydrogel. In certain embodiments, the first hydrogel comprises a porogen that degrades leaving a pore in its place. For example, the first hydrogel is a porogen and the resulting pore after degradation in situ is within 25% of the size of the initial porogen, e.g., within 20%, within 15%, or within 10% of the size of the initial porogen. Preferably, the resulting pore is within 5% of the size of the initial porogen. The first hydrogel may degrade faster than the second hydrogel due to the difference in their physical, chemical, and/or biological properties. In certain embodiments, the first hydrogel degrades more rapidly than the second hydrogel, because the first hydrogel is more soluble in water (comprises a lower solubility index). In certain embodiments, the first hydrogel degrades more rapidly because it is cross-linked to protease-mediated degradation motifs as described in U.S. Patent Publication US2005/0119762A1, the content of which is incorporated herein by reference).

In certain embodiments, the molecular mass of the polymers used to form the first hydrogel composition (a porogen) are approximately 50 kilodaltons (kDa), and the molecular mass of the polymers used to form the second hydrogel composition (bulk) comprises approximately 250 kDa. A shorter polymer (e.g. that of a porogen) degrades more quickly compared to that of a longer polymer (e.g., that of the bulk composition). In certain embodiments, a composition is modified to render it more hydrolytically degradable by virtue of the presence of sugar groups (e.g., approximately 3-10% sugar of an alginate composition). In certain embodiments, the porogen hydrogel is chemically modified, such as oxidized, to render it more susceptible to degradation. In some embodiments, the porogen hydrogel is more enzymatically degradable compared to the bulk hydrogel. The composite (first and second hydrogel) composition is permeable to bodily fluids, e.g., such as enzyme which gain access to the composition to degrade the porogen hydrogel. In some embodiments, the second hydrogel is cross-linked around the first hydrogel, i.e., the porogens (first hydrogel) are completely physically entrapped in the bulk (second) hydrogel.

The click reagents disclosed herein can be provided in the bulk hydrogel or the porogen hydrogel. In exemplary embodiments, the click reagents, e.g., unnatural sugars, polymers, and/or nanoparticles, are provided in the bulk hydrogel.

In certain embodiments, hydrogel micro-beads (“porogens”) are formed. Porogens are encapsulated into a “bulk” hydrogel that is either non-degradable or which degrades at a slower rate compared to the porogens. Immediately after hydrogel formation, or injection into the desired site in vivo, the composite material lacks pores. Subsequently, porogen degradation causes pores to form in situ. The size and distribution of pores are controlled during porogen formation, and mixing with the polymers which form the bulk hydrogel.

In some embodiments, the polymer utilized in the pore-forming scaffolds are naturally-occurring or synthetically made. In one example, both the porogens and bulk hydrogels are formed from alginate. “Alginate” as that term is used here, refers to any number of derivatives of alginic acid (e.g., calcium, sodium or potassium salts, or propylene glycol alginate). See, e.g., WO1998012228A1, hereby incorporated by reference.

In certain embodiments, the alginate polymers suitable for porogen formation have a molecular weight from 5,000 to 500,000 Daltons. The polymers are optionally further modified (e.g., by oxidation with sodium periodate, (Bouhadir et al., 2001, Biotech. Prog. 17:945-950, hereby incorporated by reference), to facilitate rapid degradation. In the certain embodiments, the polymers were crosslinked by extrusion through a nebulizer with co-axial airflow into a bath of divalent cation (for example, Ca2+ or Ba2+) to form hydrogel micro-beads. The higher the airflow rate, the lower the porogen diameter.

In some embodiments, the porogen hydrogel microbeads contain oxidized alginate. For example, the porogen hydrogel can contain 1%-50% oxidized alginate. In exemplary embodiments, the porogen hydrogel can contain 1-10% oxidized alginate. In one embodiment, the porogen hydrogel contains 7.5% oxidized alginate.

In certain embodiments, the concentration of divalent ions used to form porogens may vary from 5 to 500 mM, and the concentration of polymer from 1% to 5% by weight. However, any method which produces porogens that are significantly smaller than the bulk phase is suitable. Porogen chemistry can further be manipulated to produce porogens that have a some interaction with host proteins and cells, or to inhibit this interaction.

The alginate polymers suitable for formation of the bulk hydrogel have a Dalton molecular weight from 5,000 to 500,000 Da. The polymers may be further modified (for example, by oxidation with sodium periodate), to facilitate degradation, as long as the bulk hydrogel degrades more slowly than the porogen. The polymers may also be modified to present biological cues to control cell responses (e.g., integrin binding adhesion peptides such as RGD). Either the porogens or the bulk hydrogel may also encapsulate bioactive factors such as oligonucleotides, growth factors or drugs to further control cell responses. The concentration of divalent ions used to form the bulk hydrogel may vary from 5 to 500 mM, and the concentration of polymer from 1% to 5% by weight. The elastic modulus of the bulk polymer is tailored for its purpose, e.g., to recruit immune cells.

Methods relevant to generating the hydrogels described herein include the following. Bouhadir et al. Polymer 1999; 40: 3575-84 (incorporated herein by reference) describes the oxidation of alginate with sodium periodate, and characterizes the reaction. Bouhadir et al. Biotechnol. Prog. 2001; 17: 945-50 (incorporated herein by reference) describes oxidation of high molecular weight alginate to form alginate dialdehyde (alginate dialdehyde is high M_{w} alginate in which a certain percent, (e.g., 5%), of sugars in alginate are oxidized to form aldehydes), and application to make hydrogels degrade rapidly. Kong et al. Polymer 2002; 43: 6239-46 (incorporated herein by reference) describes the use of gamma-irradiation to reduce the weight-averaged molecular weight (M_{w}) of guluronic acid (GA) rich alginates without substantially reducing GA content (e.g., the gamma irradiation selectively attacks mannuronic acid, MA blocks of alginate). Alginate is comprised of GA blocks and MA blocks, and it is the GA blocks that give alginate its rigidity (elastic modulus). Kong et al. Polymer 2002; 43: 6239-46 (incorporated herein by reference) shows that binary combinations of high M_{w}, GA rich alginate with irradiated, low M_{w}, high GA alginate crosslinks with calcium to form rigid hydrogels, but which degrade more rapidly and also have lower solution viscosity than hydrogels made from the same overall weight concentration of only high M_{w}, GA rich alginate. Alsberg et al. J Dent Res 2003; 82(11): 903-8 (incorporated herein by reference) describes degradation profiles of hydrogels made from irradiated, low M_{w}, GA-rich alginate, with application in bone tissue engineering. Kong et al. Adv. Mater 2004; 16(21): 1917-21 (incorporated herein by reference) describes control of hydrogel degradation profile by combining gamma irradiation procedure with oxidation reaction, and application to cartilage engineering.

Techniques to control degradation of hydrogen biomaterials are well known in the art. For example, Lutolf M P et al. Nat Biotechnol. 2003; 21: 513-8 (incorporated herein by reference) describes poly(ethylene glycol) based materials engineered to degrade via mammalian enzymes (MMPs). Bryant S J et al. Biomaterials 2007; 28(19): 2978-86 (U.S. Pat. No. 7,192,693 B2; incorporated herein by reference) describes a method to produce hydrogels with macro-scale pores. A pore template (e.g., poly-methylmethacrylate beads) is encapsulated within a bulk hydrogel, and then acetone and methanol are used to extract the porogen while leaving the bulk hydrogel intact. Silva et al. Proc. Natl. Acad. Sci USA 2008; 105(38): 14347-52 (incorporated herein by reference; US 2008/0044900) describes deployment of endothelial progenitor cells from alginate sponges. The sponges are made by forming alginate hydrogels and then freeze-drying them (ice crystals form the pores). Ali et al. Nat Mater 2009 (incorporated herein by reference) describes the use of porous scaffolds to recruit dendritic cells and program them to elicit anti-tumor responses. Huebsch et al. Nat Mater 2010; 9: 518-26 (incorporated herein by reference) describes the use of hydrogel elastic modulus to control the differentiation of encapsulated mesenchymal stem cells.

In some embodiments, the scaffold composition comprises open interconnected macropores. Alternatively or in addition, the scaffold composition comprises a pore-forming scaffold composition. In certain embodiments, the pore-forming scaffold composition may comprise a sacrificial porogen hydrogel and a bulk hydrogel, wherein the pore-forming scaffold composition lacks macropores. For example, the sacrificial porogen hydrogel may degrade at least 10% faster than the bulk hydrogel leaving macropores in its place following administration of said pore-forming scaffold into a subject. In some embodiments, the sacrificial porogen hydrogel is in the form of porogens that degrade to form said macropores. For example, the macropores may comprise pores having a diameter of, e.g., about 10-400 μm.

### Mesoporous Silica Rods

In some embodiments, the scaffold device comprises mesoporous silica rods. Injectable mesoporous silica rods randomly self-assemble to form a 3D scaffold structure in vivo. The 3D scaffold structure comprises micro spaces that allow for immune cell (e.g., dendritic cell) infiltration and/or trafficking. As with other scaffold compositions disclosed herein, the mesoporous silica rods may comprise, e.g., a click chemistry reagent of the invention alone or together with an immunostimulatory compound; a compound that attracts an immune cell to or into the delivery vehicle; a compound that induces immunogenic cell death of a tumor cell; a compound that inhibits T-cell or dendritic cell suppression; a compound that inhibits an immune-inhibitory protein; or an antigen, or any combination thereof. In some embodiments, the mesoporous silica rod itself serves as an immunostimulatory compound.

In some embodiments, the rods comprise pores of between 1-50 nm in diameter, e.g; pores comprising within the range about 1-50, 2-50, 3-50, 4-50, 5-50, 6-50, 7-50, 8-50, 9-10, 10-50, 15-50, 25-50, 1-25, 2-25, 3-25, 4-25, 5-25, 6-25, 7-25, 8-25, 9-25, 10-25, or 15-25 nm. In various embodiments, the length of the mesoporous silica rods ranges from 5 m to 500 μm. In one example, the rods comprise a length of 5-25 μm, e.g., 10-20 μm. In other examples, the rods comprise length of 50 m to 250 m or 80 m to 120 μm. In certain embodiments, the mesoporous silica rods comprise a length of about 25-100, 25-250, 25-500, 50-250, or 50-500 μm, or a length of at least about 5, 10, 15, 20, 25, 30, 35, 40, 45, 50, 75, or 100 m but no more than about 500 μm.

In some embodiments, the mesoporous silica rods comprise a length of about 100 nm, 150 nm, 200 nm, 250 nm, 300 nm, 350 nm, 400 nm, 450 nm, 500 nm, 600 nm, 700 nm, 800 nm, 900 nm, 100-250 nm, 250-500 nm, 500-750 nm, 750-1000 nm, 1 μm, 2 μm, 3 μm, 4 μm, 5 μm, 6 μm, 7 μm, 8 μm, 9 μm, 10 μm, 15 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, 150 μm, 200 μm, 250 μm, 300 μm, 350 μm, 400 μm, 450 μm, 500 μm, 1-5 μm, 1-500 μm, 5-500 μm, 25-50 μm, 25-100 μm, 50-100 μm, 25-500 μm, or 50-500 μm. In certain embodiments, the mesoporous silica rods comprise of length from 100 nm, 150 nm, 200 nm, 250 nm, 300 nm, 350 nm, 400 nm, 450 nm, 500 nm, 600 nm, 700 nm, 800 nm, 900 nm, 100-250 nm, 250-500 nm, 500-750 nm, 750-1000 nm, I μm, 2 μm, 3 μm, 4 μm, 5 μm, 6 μm, 7 μm, 8 μm, 9 μm, 10 μm, 15 μm, 25 μm, 30 μm, 35 μm, 40 μm, 45 μm, or 50 m to 55 μm, 60 μm, 65 μm, 70 μm, 75 μm, 80 μm, 85 μm, 90 μm, 95 μm, 100 μm, 150 μm, 200 μm, 250 μm, 300 μm, 350 μm, 400 μm, 450 μm, or 500 μm. In various embodiments, the mesoporous silica rods comprise a length of about or at least about any of 100 nm, 150 nm, 200 nm, 250 nm, 300 nm, 350 nm, 400 nm, 450 nm, 500 nm, 600 nm, 700 nm, 800 nm, 900 nm, 100-250 nm, 250-500 nm, 500-750 nm, 750-1000 nm, 1 μm, 2 μm, 3 μm, 4 μm, 5 μm, 6 μm, 7 μm, 8 μm, 9 μm, 10 μm, 15 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, 150 μm, 200 μm, 250 μm, 1-500 μm, 5-500 μm, 25-50 μm, 25-100 μm, 50-100 μm, 25-500 μm, or 50-500 m but less than 550 μm. In some embodiments, the mesoporous silica rods comprise a diameter of about or at least about any of 75 nm, 100 nm, 150 nm, 200 nm, 250 nm, 300 nm, 350 nm, 400 nm, 450 nm, 500 nm, 600 nm, 700 nm, 800 nm, 900 nm, 100-1000 nm, 100-500 nm, 100-250 nm, 250-500 nm, 500-750 nm, or 750-1000 nm, with the proviso that mesoporous silica rods comprise a length that is at least 10% greater than the diameter thereof. In certain embodiments, the mesoporous silica rods comprise a diameter from 75 nm, 100 nm, 150 nm, 200 nm, 250 nm, 300 nm, 350 nm, 400 nm, 450 nm, or 500 nm to 600 nm, 700 nm, 800 nm, 900 nm, or 1000 nm. In some embodiments, the mesoporous silica rods comprise a length that is at least about 10, 20, 25, 30, 35, 40, 45, 50, 55, 60, 65, 70, 75, 80, 85, 90, 95, 100, or 150% greater than the diameter of the mesoporous silica rods. In some embodiments, the mesoporous silica rods comprise a length that is at least about 2, 3, 4, 5, 6, 7, 8, 9, 10, 20, 30, 40, 50, 60, 70, 80, 90, 100, 150, 200, 250, 300, 400, or 500 times the diameter of the mesoporous silica rods. In certain embodiments, the mesoporous silica rods comprise pores having a diameter of about or at least about 1, 2, 3, 4, 5, 6, 7, 8, 9, 10, 15, 20, 25, 30, 35, 40, 45, or 50 nm, or about 1-10, 1-15, 1-5, 2-5, 2-10, 3-10, 4-10, 5-10, 5-15, or 10-25 nm. In certain embodiments, the mesoporous silica rods are 80 to 120 m in length. For example, the mesoporous silica rods may comprise (a) pores having a diameter of between 2-50 nm, 3-50 nm, 5-50 nm, 5-25 nm, 5-10 nm; and/or (b) a length of about 5-25 μm, 80 to 120 μm. In some embodiments, the mesoporous silica rods may comprise a combination of rods with different lengths and/or rods with range of different sizes (e.g., within one of the ranges disclosed above or 1, 2, 3, 4, 5 or more of the ranges disclosed above). In some embodiments, rods with a length of about 100 nm, 150 nm, 200 nm, 250 nm, 300 nm, 350 nm, 400 nm, 450 nm, 500 nm, 600 nm, 700 nm, 800 nm, 900 nm, 100-250 nm, 250-500 nm, 500-750 nm, or 750-1000 nm are combined with rods having a length of about 5 μm, 6 μm, 7 μm, 8 μm, 9 μm, 10 μm, 15 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, 150 μm, 200 μm, 250 μm, 300 μm, 350 μm, 400 μm, 450 μm, 500 μm, 5-500 μm, 25-50 μm, 25-100 μm, 50-100 μm, 25-500 μm, or 50-500 μm. In certain embodiments, the rods have a width of about 0.5 μm, 1 μm, 1.5 μm, 2 μm, 2.5 μm, 3 μm, 3.5 μm, 4 μm, 4.5 μm, 5 μm, 5.5 μm, 6 μm, 6.5 μm, 7 μm, 7.5 μm, 8 μm, 8.5 μm, 9 μm, 9.5 μm, 10 μm, 11 μm, 12 μm, 13 μm, 14 μm, 15 μm, 16 μm, 17 μm, 18 μm, 19 μm, 20 μm, 1-20 μm, 1-10 μm, 5-10 μm, 1-5 μm, 0.5-20 μm, 7.5-12.5 μm, or 5-15 μm.

In some embodiments, one set of rods is small enough to be phagocytosed by immune cells such as dendritic cells or macrophages, and another set of rods is too big to be phagocytosed by the immune cells. In various embodiments, rods having different antigens or other compounds disclosed herein are mixed. Thus, provided herein are mixtures of 1, 2, 3, 4, 5, 6, 7, 8, 9, 10 or more classes of mesoporous silica rods, with each class of rods having a different antigen (e.g., antigenic peptide, such as a purified peptide). For example, a mixture may comprise a first class of rods comprising a first antigen, a second class of rods comprising a second antigen, a third class of rods comprising a third antigen, and so on. A mixture of rods may have the same or similar sizes or range of sizes, or may include one or more rods with a particular antigen or antigens (e.g., rods small enough to be phagocytosed) and another one or more rods with another antigen or antigens (e.g., rods too big to be phagocytosed). In certain embodiments, the rods that are too big to be phagocytosed form scaffolds upon administration (e.g., injection) into a subject. Injectable mesoporous silica rods randomly self-assemble to form a 3 dimensional (3D) scaffold in vivo. This system is designed such that it recruits and transiently houses immune cells (such as dendritic cells), and contact them with a click chemistry reagent of the invention. After recruitment and temporary housing or presence of the cells in the structure, these immune cells migrate out of the device structure and homed to a lymph node. Thus, the composition is one in which cells traffic/circulate in and out of, their status of immune activation being altered/modulated as a result of the trafficking through the device. In various embodiments, the mesoporous silica rods are suspended in an aqueous solution, such as a buffer [e.g., phosphate buffered saline (PBS), Hank's balanced salt solution (HBSS), or another physiologically (e.g., pharmaceutically acceptable) buffer] for injection. In some embodiments, the mesoporous silica rods are injected in water. Mesoporous silica rods may be injected in a variety of concentrations. In some embodiments, the rods are injected at a concentration of about 1 mg/ml, 2 mg/ml, 3 mg/ml, 4 mg/ml, 5 mg/ml, 6 mg/ml, 7 mg/ml, 8 mg/ml, 9 mg/ml, 10 mg/ml, 11 mg/ml, 12 mg/ml, 13 mg/ml, 14 mg/ml, 15 mg/ml, 16 mg/ml, 17 mg/ml, 18 mg/ml, 19 mg/ml, 20 mg/ml, 21 mg/ml, 22 mg/ml, 23 mg/ml, 24 mg/ml, 25 mg/ml, 30 mg/ml, 35 mg/ml, 40 mg/ml, 45 mg/ml, 50 mg/ml, 55 mg/ml, 60 mg/ml, 10-40 mg/ml, 20-35 mg/ml, 20-40 mg/ml, 25-35 mg/ml, 25-50 mg/ml, 25-45 mg/ml, 25-30 mg/ml, 30-50 mg/ml, 1-30 mg/ml, 1-40 mg/ml, 1-50 mg/ml, 1-60 mg/ml, 5-50 mg/ml, or 5-60 mg/ml.

### Chemoattractants

The device of the present invention can comprise a chemoattractant for cells. The term “chemoattractant,” as used herein, refers to any agent that attracts a motile cell, such as immune cells. To enable sustained release from the scaffold, the chemoattractant can, in some embodiments, be coupled to nanoparticles, e.g., gold nanoparticles. In certain embodiments, the chemoattractant for immune cells is a growth factor or cytokine. In some embodiments, the chemoattractant is a chemokine. Exemplary chemokines include, but are not limited to, CC chemokines, CXC chemokines, C chemokines, CX₃C chemokines. Exemplary cytokines include, but are not limited to, interleukin, lymphokines, monokines, interferons, and colony stimulating factors. All known growth factors are encompassed by the compositions, methods, and devices of the present invention. Exemplary growth factors include, but are not limited to, transforming growth factor beta (TGF-β), granulocyte-colony stimulating factor (G-CSF), granulocyte-macrophage colony stimulating factor (GM-CSF), nerve growth factor (NGF), neurotrophins, Platelet-derived growth factor (PDGF), erythropoietin (EPO), thrombopoietin (TPO), myostatin (GDF-8), growth differentiation factor-9 (GDF9), acidic fibroblast growth factor (aFGF or FGF-1), basic fibroblast growth factor (bFGF or FGF-2), epidermal growth factor (EGF), hepatocyte growth factor (HGF). In some embodiments, the device includes a chemoattractant for immune cells. In some embodiments, the device comprises a compound that attracts an immune cell to or into the device, wherein the immune cell comprises a macrophage, a T-cell, a B-cell, a natural killer (NK) cell, a regulatory T (Treg) cell, and/or a dendritic cell. Non-limiting examples of compounds useful for attracting an immune cell to or into the device comprises granulocyte-macrophage colony stimulating factor (GM-CSF), an FMS-like tyrosine kinase 3 ligand (Fit3L), chemokine (C—C motif) ligand 19 (CCL-19), chemokine (C—C motif) ligand 20 (CCL20), chemokine (C—C motif) ligand 21 (CCL-21), a N-formyl peptide, fractalkine, monocyte chemotactic protein-1, and macrophage inflammatory protein-3 (MIP-3a). The present invention encompasses cytokines as well as growth factors for stimulating dendritic cell activation. Exemplary cytokines include, but are not limited to, IL-1, IL-2, IL-3, IL-4, IL-5, IL-6, IL-8, IL-10, IL-12 IL-15, IL-17, IL-18, TNF-α, IFN-γ, and IFN-α.

In certain embodiments, the chemoattractant for immune cells is Granulocyte-macrophage colony-stimulating factor (GM-CSF). Granulocyte-macrophage colony-stimulating factor (GM-CSF) is a protein secreted by macrophages, T cells, mast cells, endothelial cells and fibroblasts. Specifically, GM-CSF is a cytokine that functions as a white blood cell growth factor. GM-CSF stimulates stem cells to produce granulocytes and monocytes. Monocytes exit the blood stream, migrate into tissue, and subsequently mature into macrophages.

In some embodiments, the device can comprise and release GM-CSF polypeptides to attract host DCs to the device. Contemplated GM-CSF polypeptides are isolated from endogenous sources or synthesized in vivo or in vitro. Endogenous GM-CSF polypeptides may be isolated from healthy human tissue. Synthetic GM-CSF polypeptides are synthesized in vivo following transfection or transformation of template DNA into a host organism or cell, e.g., a mammalian or human cell line. Alternatively, synthetic GM-CSF polypeptides are synthesized in vitro by polymerase chain reaction (PCR) or other art-recognized methods Sambrook, J., Fritsch, E. F., and Maniatis, T., Molecular Cloning: A Laboratory Manual. Cold Spring Harbor Laboratory Press, NY, Vol. 1, 2, 3 (1989), herein incorporated by reference).

In certain embodiments, GM-CSF polypeptides may be recombinant. In some embodiments, GM-CSF polypeptides are humanized derivatives of mammalian GM-CSF polypeptides. Exemplary mammalian species from which GM-CSF polypeptides are derived include, but are not limited to, mouse, rat, hamster, guinea pig, ferret, cat, dog, monkey, or primate. In some embodiments, GM-CSF is a recombinant human protein (PeproTech, Catalog #300-03). In some embodiments, GM-CSF is a recombinant murine (mouse) protein (PeproTech, Catalog #315-03). In some embodiments, GM-CSF is a humanized derivative of a recombinant mouse protein.

In certain embodiments, GM-CSF polypeptides may be modified to increase protein stability in vivo. In certain embodiments, GM-CSF polypeptides may be engineered to be more or less immunogenic. Endogenous mature human GM-CSF polypeptides are glycosylated, reportedly, at amino acid residues 23 (leucine), 27 (asparagine), and 39 (glutamic acid) (see U.S. Pat. No. 5,073,627). In certain embodiments, GM-CSF polypeptides of the present invention may be modified at one or more of these amino acid residues with respect to glycosylation state.

The chemoattractant for immune cells may recruit immune cells to the scaffolds of the present invention. Immune cells include cells of the immune system that are involved in immune response. Exemplary immune cells includes, but not limited to, T cells, B cells, leucocytes, lymphocytes, antigen presenting cells, dendritic cells, neutrophils, eosinophils, basophils, monocytes, macrophages, histiocytes, mast cells, and microglia.

In certain embodiments, the chemoattractant for immune cells recruits dendritic cells (DCs) to the scaffold of the present invention. Dendritic cells (DCs) are immune cells within the mammalian immune system and are derived from hematopoietic bone marrow progenitor cells. More specifically, dendritic cells can be categorized into lymphoid (or plasmacytoid) dendritic cell (pDC) and myeloid dendritic cell (mDC) subdivisions having arisen from a lymphoid (or plasmacytoid) or myeloid precursor cell, respectively. From the progenitor cell, regardless of the progenitor cell type, an immature dendritic cell is born. Immature dendritic cells are characterized by high endocytic activity and low T-cell activation potential. Thus, immature dendritic cells constitutively sample their immediate surrounding environment for pathogens. Exemplary pathogens include, but are not limited to, a virus or a bacteria. Sampling is accomplished by pattern recognition receptors (PRRs) such as the toll-like receptors (TLRs). Dendritic cells activate and mature once a pathogen is recognized by a pattern recognition receptor, such as a toll-like receptor.

Mature dendritic cells not only phagocytose pathogens and break them down, but also, degrade their proteins, and present pieces of these proteins, also referred to as antigens, on their cell surfaces using MHC (Major Histocompatibility Complex) molecules (Classes I, II, and III). Mature dendritic cells also upregulate cell-surface receptors that serve as co-receptors for T-cell activation. Exemplary co-receptors include, but are not limited to, CD80, CD86, and CD40. Simultaneously, mature dendritic cells upregulate chemotactic receptors, such as CCR7, that allows the cell to migrate through the blood stream or the lymphatic system to the spleen or lymph node, respectively.

Dendritic cells are present in external tissues that are in contact with the external environment such as the skin (dendritic cells residing in skin are also referred to as Langerhans cells). Alternatively, dendritic cells are present in internal tissues that are in contact with the external environment such as linings of the nose, lungs, stomach, and intestines. Finally, immature dendritic cells reside in the blood stream. Once activated, dendritic cells from all off these tissues migrate to lymphoid tissues where they present antigens and interact with T cells and B cells to initiate an immune response. One signaling system of particular importance for the present invention involves the chemokine receptor CCR7 expressed on the surface of dendritic cells and the chemokine receptor ligand CCL19 secreted by lymph node structures to attract migrating mature dendritic cells toward high concentrations of immune cells. Exemplary immune cells activated by contact with mature dendritic cells include, but are not limited to, helper T cells, killer T cells, and B cells. Although multiple cell types within the immune system present antigens, including macrophages and B lymphocytes, dendritic cells are the most potent activators of all antigen-presenting cells.

Dendritic cells earned their name from the characteristic cell shape comprising multiple dendrites extending from the cell body. The functional benefit of this cell shape is a significantly increased cell surface and contact area to the surroundings compared to the cell volume. Immature dendritic cells sometimes lack the characteristic dendrite formations and are referred to as veiled cells. Veiled cells possess large cytoplasmic veils rather than dendrites.

### Adjuvants

In certain embodiments, the device of the present invention comprises an adjuvant. The term “adjuvant”, as used herein, refers to compounds that can be added to vaccines to stimulate immune responses against antigens. Adjuvants may enhance the immunogenicity of highly purified or recombinant antigens. Adjuvants may reduce the amount of antigen or the number of immunizations needed to protective immunity. For example, adjuvants may activate antibody-secreting B cells to produce a higher amount of antibodies. Alternatively, adjuvants can act as a depot for an antigen, present the antigen over a longer period of time, which could help maximize the immune response and provide a longer-lasting protection. Adjuvants may also be used to enhance the efficacy of a vaccine by helping to modify the immune response to particular types of immune system cells, for example, by activating T cells instead of antibody-secreting B cells depending on the purpose of the vaccine. Adjuvants are also used in the production of antibodies from immunized animals (Petrovskyl et al, 2002, Immunology and Cell Biology 82: 488-496).

Adjuvants can be classified according to their source, mechanism of action or physicochemical properties. For example, adjuvants can be classified into three groups: (i) active immunostimulants, being substances that increase the immune response to the antigen; (ii) carriers, being immunogenic proteins that provide T-cell help; and (iii) vehicle adjuvants, being oil emulsions or liposomes that serve as a matrix for antigens as well as stimulating the immune response (Edelman R. 1992, AIDS Res. Hum. Retroviruses 8: 1409-11). An alternative adjuvant classification divides adjuvants according to administration route, namely mucosal or parenteral. A third classification divides adjuvants into alum salts and other mineral adjuvants; tensoactive agents; bacterial derivatives; vehicles and slow release materials or cytokines (Byars et al., 1990, Laboratory Methods in Immunology: 39-51). A fourth classification divides adjuvants into the following groups: gel-based adjuvants, tensoactive agents, bacterial products, oil emulsions, particulated adjuvants, fusion proteins or lipopeptides (Jennings R et al., 1998, Dev. Biol. Stand, 92: 19-28).

The device of the present invention may comprise one or more adjuvants. Adjuvants suitable for use in the present invention include, but are not limited to, mineral salt-based adjuvants such as alum-based adjuvants, calcium-based adjuvants, iron-based adjuvants, zirconium-based adjuvants; particulate adjuvants; mucosal adjuvants; tensoactive adjuvants; bacteria-derived adjuvants; oil-based adjuvants; cytokines, liposome adjuvants, polymeric microsphere adjuvants, carbohydrate adjuvants.

Exemplary adjuvants include, but are not limited to, aluminium hydroxide, aluminum phosphate, calcium phosphate, Quil A, Quil A derived saponin QS-21, or other types of saponins, Detox, ISCOMs, cell wall peptidoglycan or lipopolysaccharide of Gram-negative bacteria, trehalose dimycolate, bacterial nucleic acids such as DNA containing CpG motifs, FIA, Montanide, Adjuvant 65, Freund's complete adjuvant, Freund's incomplete adjuvant, Lipovant, interferon, granulocyte-macrophage colony stimulating factor (GM-CSF), AS03, AS04, IL-1, IL-2, IL-3, IL-4, IL-5, IL-6, IL-7, IL-8, IL-10, IL-12, IL-15, IL-17, IL-18, STING, Toll-like receptor ligand, CD40L, ovalbumin (OVA), monophosphoryl lipid A (MPL), polyinosinic:polycytidylic acid (poly(I:C)), a combination of LPS (or MPLA) and OxPAPC, MF59, N-acetyl muramyl-L-alanyl-D-isoglutamine (MDP), poly (DL-lactide-coglycolide) microspheres, paraffin oil, squalene, virosome, gamma inulin, glucans, dextrans, lentinans, glucomannans and galactomannans, pathogen-associated molecular patterns (PAMPs), damage-associated molecular pattern molecules (DAMPs), antibodies against immune suppressive molecules (e.g., antibody or antagonist against transforming growth factor (TGF)-beta, A2aR antagonists), Freund's complete adjuvant, Freund's incomplete adjuvant, lipopolysaccharides (LPS), Fas ligand, Trail, lymphotactin, Mannan (M-FP), APG-2, Hsp70 and Hsp90.

In certain embodiments, the device of the present invention comprises an agent that activates and matures recruited immune cells. In some embodiments, the agent is a toll-like receptor (TLR) ligand.

TLRs are a class of single transmembrane domain, non-catalytic, receptors that recognize structurally conserved molecules referred to as pathogen-associated molecular patterns (PAMPs). PAMPs are present on microbes and are distinguishable from host molecules. TLRs are present in all vertebrates. Thirteen TLRs (referred to as TLRs1-13, consecutively) have been identified in humans and mice. Human TLRs comprise TLRs 1-10.

TLRs and interleukin-1 (IL-1) receptors comprise a receptor superfamily the members of which all share a TIR domain (Toll-IL-1 receptor). TIR domains exist in three varieties with three distinct functions. TIR domains of subgroup 1 are present in receptors for interleukins produced by macrophages, monocytes, and dendritic cells. TIR domains of subgroup 2 are present in classical TLRs which bind directly or indirectly to molecules of microbial origin. TIR domains of subgroup 3 are present in cytosolic adaptor proteins that mediate signaling between proteins comprising TIR domains of subgroups 1 and 2.

TLR ligands comprise molecules that are constantly associated with and highly specific for a threat to the host's survival such as a pathogen or cellular stress. TLR ligands are highly specific for pathogens and not the host. Exemplary pathogenic molecules include, but are not limited to, lipopolysaccharides (LPS), lipoproteins, lipoarabinomannan, flagellin, double-stranded RNA, and unmethylated CpG islands of DNA.

All known TLR ligands found either on a cell surface or an internal cellular compartment are encompassed by the compositions, methods, and devices of the present invention. Exemplary TLR ligands include, but are not limited to, triacyl lipoproteins (TLR1); lipoproteins, gram positive peptidoglycan, lipteichoic acids, fungi, and viral glycoproteins (TLR2); double-stranded RNA, poly I:C (TLR 3); lipopolysaccaride, viral glycoproteins (TLR 4); flagellin (TLR5); diacyl lipoproteins (TLR6); small synthetic compounds, single-stranded RNA (TLR7 and TLR 8); unmethylated CpG DNA (TLR9); Profilin (TLR11). Also included as TRL ligands are host molecules like fibronectin and heat shock proteins (HSPs). Host TLR ligands are also encompassed by the present invention. The role of TLRs in innate immunity and the signaling molecules used to activate and inhibit them are known in the art (for a review, see Holger K. Frank B., Hessel E., and Coffman R L. Therapeutic targeting of innate immunity with Toll-like receptor agonists and antagonists. Nature Medicine 13, 552-559 (2007), the content of which is herein incorporated by reference).

CpG sites are regions of deoxyribonucleic acid (DNA) where a cysteine nucleotide occurs next to a guanine nucleotide in the linear sequence of bases along its length (the “p” represents the phosphate linkage between them and distinguishes them from a cytosine-guanine complementary base pairing). CpG sites play a pivotal role in DNA methylation, which is one of several endogenous mechanisms cells use to silence gene expression. Methylation of CpG sites within promoter elements can lead to gene silencing. In the case of cancer, it is known that tumor suppressor genes are often silences while oncogenes, or cancer-inducing genes, are expressed. Importantly, CpG sites in the promoter regions of tumor suppressor genes (which prevent cancer formation) have been shown to be methylated while CpG sites in the promoter regions of oncogenes are hypomethylated or unmethylated in certain cancers. The TLR-9 receptor binds unmethylated CpG sites in DNA.

In certain embodiments, the device of present invention comprises a cytosine-guanosine dinucleotides and oligonucleotides (CpG-ODN). Contemplated CpG oligonucleotides may be isolated from endogenous sources or synthesized in vivo or in vitro. Exemplary sources of endogenous CpG oligonucleotides include, but are not limited to, microorganisms, bacteria, fungi, protozoa, viruses, molds, or parasites. In some embodiments, endogenous CpG oligonucleotides are isolated from mammalian benign or malignant neoplastic tumors. In some embodiments, synthetic CpG oligonucleotides are synthesized in vivo following transfection or transformation of template DNA into a host organism. In certain embodiments, Synthetic CpG oligonucleotides are synthesized in vitro by polymerase chain reaction (PCR) or other art-recognized methods (Sambrook, J., Fritsch, E. F., and Maniatis, T., Molecular Cloning: A Laboratory Manual. Cold Spring Harbor Laboratory Press, NY, Vol. 1, 2, 3 (1989), herein incorporated by reference).

CpG oligonucleotides are presented for cellular uptake by dendritic cells. In some embodiments, naked CpG oligonucleotides are used. The term “naked” is used to describe an isolated endogenous or synthetic polynucleotide (or oligonucleotide) that is free of additional substituents. In some embodiments, CpG oligonucleotides are bound to one or more compounds to increase the efficiency of cellular uptake. In some embodiments, CpG oligonucleotides are bound to one or more compounds to increase the stability of the oligonucleotide within the scaffold and/or dendritic cell.

In certain embodiments, CpG oligonucleotides are condensed prior to cellular uptake. In some embodiments, CpG oligonucleotides are condensed using polyethylimine (PEI), a cationic polymer that increases the efficiency of cellular uptake into dendritic cells.

CpG oligonucleotides can be divided into multiple classes. For example, exemplary CpG-ODNs encompassed by compositions, methods and devices of the present invention are stimulatory, neutral, or suppressive. The term “stimulatory” used herein is meant to describe a class of CpG-ODN sequences that activate TLR9. The term “neutral” used herein is meant to describe a class of CpG-ODN sequences that do not activate TLR9. The term “suppressive” used herein is meant to describe a class of CpG-ODN sequences that inhibit TLR9. The term “activate TLR9” describes a process by which TLR9 initiates intracellular signaling.

Simulatory CpG-ODNs can further be divided into three types A, B and C, which differ in their immune-stimulatory activities. Type A stimulatory CpG ODNs are characterized by a phosphodiester central CpG-containing palindromic motif and a phosphorothioate 3′ poly-G string. Following activation of TLR9, these CpG ODNs induce high IFN-α production from plasmacytoid dendritic cells (pDC). Type A CpG ODNs weakly stimulate TLR9-dependent NF-κB signaling.

Type B stimulatory CpG ODNs contain a full phosphorothioate backbone with one or more CpG dinucleotides. Following TLR9 activation, these CpG-ODNs strongly activate B cells. In contrast to Type A Cpg-ODNs, Type B CpG-ODNs weakly stimulate IFN-α secretion.

Type C stimulatory CpG ODNs comprise features of Types A and B. Type C CpG-ODNs contain a complete phosphorothioate backbone and a CpG containing palindromic motif. Similar to Type A CpG ODNs, Type C CpG ODNs induce strong IFN-α production from pDC. Simlar to Type B CpG ODNs, Type C CpG ODNs induce strong B cell stimulation.

Exemplary stimulatory CpG ODNs comprise, but are not limited to, ODN 1585, ODN 1668, ODN 1826, ODN 2006, ODN 2006-G5, ODN 2216, ODN 2336, ODN 2395, ODN M362 (all InvivoGen). The present invention also encompasses any humanized version of the preceding CpG ODNs. In one preferred embodiment, compositions, methods, and devices of the present invention comprise ODN 1826 (the sequence of which from 5′ to 3′ is tccatgacgttcctgacgtt, wherein CpG elements are bolded, SEQ ID NO: 10).

Neutral, or control, CpG ODNs that do not stimulate TLR9 are encompassed by the present invention. These ODNs comprise the same sequence as their stimulatory counterparts but contain GpC dinucleotides in place of CpG dinucleotides.

Exemplary neutral, or control, CpG ODNs encompassed by the present invention comprise, but are not limited to, ODN 1585 control, ODN 1668 control, ODN 1826 control, ODN 2006 control, ODN 2216 control, ODN 2336 control, ODN 2395 control, ODN M362 control (all InvivoGen). The present invention also encompasses any humanized version of the preceding CpG ODNs.

Suppressive CpG ODNs that inhibit TLR9 are encompassed by the present invention. Exemplary potent inhibitory sequences are (TTAGGG)₄ (ODN TTAGGG, InvivoGen, SEQ ID NO:11), found in mammalian telomeres and ODN 2088 (InvivoGen), derived from a murine stimulatory CpG ODN by replacement of 3 bases. Suppressive ODNs disrupt the colocalization of CpG ODNs with TLR9 in endosomal vesicles without affecting cellular binding and uptake. Suppressive CpG ODNs encompassed by the present invention are used to fine-tune, attenuate, reverse, or oppose the action of a stimulatory CpG-ODN. Alternatively, or in addition, compositions, methods, or devices of the present invention comprising suppressive CpG ODNs are used to treat autoimmune conditions or prevent immune responses following transplant procedures.

### Antigens

In certain embodiments, the device of the present invention comprises an antigen. The antigen can be a cancer antigen or a non-cancer antigen (e.g., a microbial antigen or a viral antigen). In one embodiment, the antigen is a polypeptide. In one embodiment, the polypeptide antigen comprises a stretch of at least 10 consecutive amino acids identical to a stretch of at least 10 consecutive amino acids of a cancer antigen, a microbial antigen, or a viral antigen. In some embodiments, the antigen is a cancer antigen. The device comprising a cancer antigen can be used to vaccinate and/or provide protective immunity to a subject to whom such a device was administered. In some embodiments, a cancer/tumor antigen is from a subject who is administered a device provided herein. In certain embodiments, a cancer/tumor antigen is from a different subject. In various embodiments, a cancer antigen is present in a cancer cell lysate. For example, the tumor cell lysate may comprise one or more lysed cells from a biopsy. In some embodiments, the cancer antigen is present on an attenuated live cancer cell. For example, the attenuated live cancer cell may be an irradiated cancer cell. Antigens may be used alone or in combination with GM-CSF, CpG-ODN sequences, or immunomodulators. Moreover, antigens can be provided simultaneously or sequentially with GM-CSF, CpG-ODN sequences, or immunomodulators.

One or more antigens may be selected based on an antigenic profile of a subject's cancer or of a pathogen. In certain embodiments, the device lacks a cancer antigen prior to administration to a subject. In some embodiments, the device comprises an immunoconjugate, wherein the immunoconjugate comprises an immunostimulatory compound covalently linked to an antigen. In various embodiments, the antigen comprises a cancer antigen, such as a central nervous system (CNS) cancer antigen, CNS germ cell tumor antigen, lung cancer antigen, leukemia antigen, acute myeloid leukemia antigen, multiple myeloma antigen, renal cancer antigen, malignant glioma antigen, medulloblastoma antigen, breast cancer antigen, prostate cancer antigen, Kaposi's sarcoma antigen, ovarian cancer antigen, adenocarcinoma antigen, or melanoma antigen. In some embodiments, treating the subject comprises reducing metastasis in the subject.

Exemplary cancer antigens encompassed by the compositions, methods, and devices of the present invention include, but are not limited to, tumor lysates extracted from biopsies, and irradiated tumor cells. Exemplary polypeptide cancer antigens include one or more of the following proteins, or fragments thereof: MAGE series of antigens (MAGE-1 is an example), MART-1/melana, tyrosinase, ganglioside, gp100, GD-2, O-acetylated GD-3, GM-2, MUC-1, Sos1, Protein kinase C-binding protein, Reverse transcriptase protein, AKAP protein, VRK1, KIAA1735, T7-1, T11-3, T11-9, Homo Sapiens telomerase ferment (hTRT), Cytokeratin-19 (CYFRA21-1), SQUAMOUS CELL CARCINOMA ANTIGEN 1 (SCCA-1), (PROTEIN T4-A), SQUAMOUS CELL CARCINOMA ANTIGEN 2 (SCCA-2), Ovarian carcinoma antigen CA125 (1A1-3B) (KIAA0049), MUCIN 1 (TUMOR-ASSOCIATED MUCIN), (CARCINOMA-ASSOCIATED MUCIN), (POLYMORPHIC EPITHELIAL MUCIN), (PEM), (PEMT), (EPISIALIN), (TUMOR-ASSOCIATED EPITHELIAL MEMBRANE ANTIGEN), (EMA), (H23AG), (PEANUT-REACTIVE URINARY MUCIN), (PUM), (BREAST CARCINOMA-ASSOCIATED ANTIGEN DF3), CTCL tumor antigen sel-1, CTCL tumor antigen se14-3, CTCL tumor antigen se20-4, CTCL tumor antigen se20-9, CTCL tumor antigen se33-1, CTCL tumor antigen se37-2, CTCL tumor antigen se57-1, CTCL tumor antigen se89-1, Prostate-specific membrane antigen, 5T4 oncofetal trophoblast glycoprotein, Orf73 Kaposi's sarcoma-associated herpesvirus, MAGE-C1 (cancer/testis antigen CT7), MAGE-B1 ANTIGEN (MAGE-XP ANTIGEN) (DAM10), MAGE-B2 ANTIGEN (DAM6), MAGE-2 ANTIGEN, MAGE-4a antigen, MAGE-4b antigen, Colon cancer antigen NY-CO-45, Lung cancer antigen NY-LU-12 variant A, Cancer associated surface antigen, Adenocarcinoma antigen ART1, Paraneoplastic associated brain-testis-cancer antigen (onconeuronal antigen MA2; paraneoplastic neuronal antigen), Neuro-oncological ventral antigen 2 (NOVA2), Hepatocellular carcinoma antigen gene 520, TUMOR-ASSOCIATED ANTIGEN CO-029, Tumor-associated antigen MAGE-X2, Synovial sarcoma, X breakpoint 2, Squamous cell carcinoma antigen recognized by T cell, Serologically defined colon cancer antigen 1, Serologically defined breast cancer antigen NY-BR-15, Serologically defined breast cancer antigen NY-BR-16, Chromogranin A; parathyroid secretory protein 1, DUPAN-2, CA 19-9, CA 72-4, CA 195, Carcinoembryonic antigen (CEA), Trp2, ovalbumin, M27, and M30. In embodiments, the antigen comprises a fragment of one or more of the following proteins. In exemplary embodiments, the fragment can comprise 10 or more consecutive amino acids identical in sequence to one or more of the foregoing proteins. In some embodiments, the fragment can comprise 10, 20, 30, 40, 50, 60, 70, 80, 90, 100, 150, 200, 250, 300, 350, 400, 450, 500, 600, 700, 800, 900, 1000 or more amino acids. In one embodiment, the fragment can comprise 10-500 amino acids.

In one embodiment, the antigen is a melanoma antigen. Exemplary melanoma antigens include, but are not limited to, tyrosinase, gp75 (tyrosinase related protein-1 (TRP-1)), gp100 (Pmel17), Melan A/MART-1, TRP-2, MAGE family, BAGE family, GAGE family, NY-ESO-1, CDK4, β-catenin, mutated introns, N-acetylglucosaminyltransferase V gene product, MUM-1, p15, gangliosides (e.g., GM2, GD2, GM3, GD3), high molecular weight chondroitin sulfate proteoglycan, p97 melanotransferrin, and SEREX antigens (e.g., D-1, SSX-2) (Hodi F S, Clin Cancer Res, Feb. 1, 2006, 12: 673-678), or fragments thereof.

In certain embodiments, the antigen comprises a non-tumor antigen such as a microbial antigen. For example, the microbial antigen may comprise a bacterial antigen, a fungal antigen, an archaean antigen, or a protozoan antigen. In some embodiments, the microbial antigen is a viral antigen, e.g., an HIV antigen or influenza antigen. In some embodiments, the antigen is from a microbe such as a bacterium, virus, protozoan, archaean, or fungus. Various embodiments relate to vaccinating against or treating a bacterial, viral, or fungal infection. In various embodiments, a delivery vehicle comprising an antigen from a pathogen. For example, a pathogen includes but is not limited to a fungus, a bacterium (e.g., Staphylococcus species, Staphylococcus aureus, Streptococcus species, Streptococcus pyogenes, Pseudomonas aeruginosa, Burkholderia cenocepacia, Mycobacterium species, Mycobacterium tuberculosis, Mycobacterium avium, Salmonella species, Salmonella typhi, Salmonella typhimurium, Neisseria species, Brucella species, Bordetella species, Borrelia species, Campylobacter species, Chlamydia species, Chlamydophila species, Clostrium species, Clostrium botulinum, Clostridium difficile, Clostridium tetani, Helicobacter species, Helicobacter pylori, Mycoplasma pneumonia, Corynebacterium species, Neisseria gonorrhoeae, Neisseria meningitidis, Enterococcus species, Escherichia species, Escherichia coli, Listeria species, Francisella species, Vibrio species, Vibrio cholera, Legionella species, or Yersinia pestis), a virus (e.g., adenovirus, Epstein-Barr virus, Hepatitis A virus, Hepatitis B virus, Hepatitis C virus, Herpes simplex virus type 1, 2, or 8, human immunodeficiency virus, influenza virus, measles, Mumps, human papillomavirus, poliovirus, rabies, respiratory syncytial virus, rubella virus, or varicella-zoster virus), a parasite or a protozoa (e.g., Entamoeba histolytica, Plasmodium, Giardia lamblia, Trypanosoma brucei, or a parasitic protozoa such as malaria-causing Plasmodium). In one embodiment, a pathogen antigen can be derived from a pathogen cell or particle described herein.

### IV. Labeling Cells with Click Reagents In Vivo

In one embodiment, the invention provides an in vivo method of labeling a cell, e.g., an immune cell, e.g., a T cell, with a click reagent. In an exemplary embodiment, the click chemistry reagent is formulated as an unnatural sugar. In an exemplary embodiment, the click chemistry reagent is formulated in a nanoparticle.

In some embodiments, the method can comprise administering to a subject a device comprising a polymer scaffold and a click reagent, as disclosed herein, and maintaining the device in the subject for a period of time sufficient for recruitment of the cell to the device. Devices comprising click reagents are disclosed herein. Any of the devices disclosed herein are suitable for use in in vivo methods of cell labeling. In exemplary embodiments, the device comprises a hydrogel scaffold containing nanoparticles comprising click chemistry reagents embedded therein. In exemplary embodiments, the device comprises a hydrogel scaffold containing unnarual sugars comprising click chemistry reagents.

Following administration, the device can be maintained in the subject for a period of time sufficient for recruitment of cells to the device. The period of time sufficient for recruitment of cells can be determined by methods including, for example, administering the device to one or more test subjects, removing the device after predetermined intervals of time, and quantifying the number of cells present in the device. In an exemplary embodiment, the cells are immune cells, e.g., dendritic cells. In one embodiment, the period of time sufficient for recruitment of cells is 2-21 days. In another embodiment, the period of time sufficient for recruitment of cells is 2-14 days. In another embodiment, the period of time sufficient for recruitment of cells is 2-10 days. In another embodiment, the period of time sufficient for recruitment of cells is 3-7 days. In another embodiment, the period of time sufficient for recruitment of cells is 3-5 days. In exemplary embodiments, the period of time sufficient for recruitment of cells is about 2, 3, 4, 5, 6, 7, 8, 9, 10, 11, 12, 13, 14, 15, 16, 17, 18, 19, 20, or 21 days, or more. In another exemplary embodiment, the period of time sufficient for recruitment of cells is about 3 days. In another exemplary embodiment, the period of time sufficient for recruitment of cells is at least 2 days, at least 3 days, at least 4 days, at least 5 days, at least 6 days, at least 7 days, at least 8 days, at least 9 days, at least 10 days, at least 14 days, at least 21 days, or more. In some embodiments, the period of time sufficient for recruitment of cells is at least about 24 hours, 48 hours, 72 hours, 96 hours, or 120 hours. In some embodiments, the period of time sufficient for recruitment of cells is about 48-96 hours. In some embodiments, the period of time sufficient for recruitment of cells is about 48-72 hours. In some embodiments, the period of time sufficient for recruitment of cells is about 72 hours.

In embodiments in which the device is a hydrogel, the hydrogel scaffold can, in some embodiments, be disrupted by application of ultrasound to the device, e.g., by application of ultrasound to the subject in the vicinity of the device. Ultrasound treatment can induce the burst release of reagents, e.g., polymers or nanoparticles, embedded in the hydrogel, by temporarily disrupting the ionic crosslinks of the gel. Accordingly, ultrasound can be applied after infiltration of cells, e.g., immune cells, into the device, to increase the availability of nanoparticles for uptake by the cells. In one embodiment, ultrasound is applied to the hydrogel after a period of time sufficient for recruitment of cells to the device. For example, ultrasound can be applied to the scaffold about 2-21 days after administration of the scaffold to a subject. In another embodiment, ultrasound is applied to the hydrogel scaffold about 2-14 days after administration of the scaffold to a subject. In another embodiment, ultrasound is applied to the hydrogel scaffold about 2-10 days after administration of the scaffold to a subject. In another embodiment, ultrasound is applied to the hydrogel scaffold about 3-7 days after administration of the scaffold to a subject. In another embodiment, ultrasound is applied to the hydrogel scaffold about 3-5 days after administration of the scaffold to a subject. In another embodiment, ultrasound is applied to the hydrogel scaffold about 2, 3, 4, 5, 6, 7, 8, 9, 10, 11, 12, 13, 14, 15, 16, 17, 18, 19, 20, or 21 days, or more, after administration of the scaffold to a subject. In another embodiment, ultrasound is applied to the hydrogel scaffold about 3 days after administration of the scaffold to a subject. In another exemplary embodiment, ultrasound is applied to the hydrogel scaffold at least 2 days, at least 3 days, at least 4 days, at least 5 days, at least 6 days, at least 7 days, at least 8 days, at least 9 days, at least 10 days, at least 14 days, at least 21 days, or more after administration of the scaffold to a subject. In some embodiments, ultrasound is applied to the hydrogel scaffold at least about 24 hours, 48 hours, 72 hours, 96 hours, or 120 hours after administration of the scaffold to a subject. In some embodiments, ultrasound is applied to the hydrogel scaffold about 48-96 hours after administration of the scaffold to a subject. In some embodiments, ultrasound is applied to the hydrogel scaffold about 48-72 hours after administration of the scaffold to a subject. In some embodiments, ultrasound is applied to the hydrogel scaffold about 72 hours after administration of the scaffold to a subject.

Ultrasound parameters, including the amplitude and duration of treatment, can be selected using standard methods. In one embodiment, the ultrasound treatment is applied at about 10%, 20%, 30%, 40%, 50%, 60%, 70%, 80%, or 90% amplitude. In an exemplary embodiment, the ultrasound treatment is applied at 20-40% amplitude. In another exemplary embodiment, the ultrasound treatment is applied at about 30% amplitude. In one embodiment, the ultrasound treatment is applied for a duration of about 1-30 minutes. For example, the ultrasound treatment can be applied for about 1, 2, 3, 4, 5, 6, 7, 8, 9, 10, 12, 15, 20, 25, or 30 minutes. In an exemplary embodiment, the ultrasound treatment is applied for about 1-5 minutes, e.g., 2-3 minutes. In one embodiment, the ultrasound treatment is applied for about 2.5 minutes.

As used herein, the term “administering,” generally refers to the placement of the compositions and/or agents described herein into a subject. Exemplary modes of administration include, but are not limited to, injection, infusion, instillation, inhalation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In preferred embodiments, the compositions are administered by injection, e.g., subcutaneous injection or intratumoral injection, or by intravenous infusion.

Administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the compounds are formulated into ointments, salves, gels, or creams as generally known in the art.

In some embodiments, administration includes implanting or injecting a device, e.g., a hydrogel, described herein in a subject.

In one embodiment, the site of administration is at or near the site of a tumor in a subject. For example, the device can be administered within 5 cm of a tumor, e.g., within 4 cm, within 3 cm, within 2 cm, or within 1 cm of a tumor in the subject. In other embodiments, the device can be administered within 10 mm of tumor, e.g., within 9 mm, within 8 mm, within 7 mm, within 6 mm, within 5 mm, within 4 mm, within 3 mm, within 2 mm, or within 1 mm of a tumor in a subject. In other embodiments, the device can be administered intratumorally.

In another embodiment, the site of administration is distal from the site of a tumor in a subject. For example, the device can be administered more than 5 cm from the site of a tumor. In one embodiment, the device is administered to a limb of a subject, e.g., to an arm or leg of a subject.

### V. Compositions and Methods for Targeting Agents to Cells Using Click Chemistry Pairs

Cells labeled with click reagents in vitro, ex vivo, or in vivo can be covalently coupled to a moiety of interest using click chemistry. For example, the cell can be contacted with a counterpart click reagent that is, in turn, attached to a moiety, thereby conjugating the moiety to the cell. The contacting can occur in vitro, ex vivo, or in vivo. Accordingly, in one embodiment, cells are labeled with a click reagent in vitro or ex vivo, and are contacted in vitro or ex vivo with a counterpart click reagent that is attached to a moiety for conjugation to the cells. In another embodiment, cells are labeled with a click reagent in vitro or ex vivo, and are contacted in vivo with a counterpart click reagent that is attached to a moiety for conjugation to the cells. In this embodiment, the contacting can be performed by administration of the counterpart click reagent attached to the moiety to a subject who comprises the click-labeled cells. In another embodiment, cells are labeled with a click reagent in vivo, and are contacted in vivo with a counterpart click reagent that is attached to a moiety for conjugation to the cells. Exemplary moieties that can be conjugated to cells in this manner are described below.

Accordingly, the present invention provides cells that include a glycoprotein-agent complex. The glycoprotein-agent complex is formed through specific or selective click reaction between a cell labeled with a click reagent and an agent coupled to a counterpart click reagent. In some embodiments, the glycoprotein-agent complex is located within the cell. In certain embodiments, the agent retains its structural integrity, function, and/or activity while residing within the cell.

In certain embodiments, the present invention provides methods to label and target a cell directly. For example, a cell may be labeled with a click reagent by contacting the cell directly with the click reagent, e.g., G400 NP. The cell labeled with a click reagent may be subsequently targeted by a counterpart click reagent, e.g., a moiety coupled to DBCO, by a direct contact with the counterpart click reagent. The advantage of direct labeling and/or targeting methods according to this disclosure includes, but is not limited to, that it does not require an intermediary, e.g., exosome.

In some embodiments, the click reagent presented on the surface of a cell, e.g., coupled to a cell surface glycoprotein, may react with its counterpart click reagent that is, in turn, attached to a moiety, thereby conjugating the moiety to the cell. Any moiety may be conjugated to the click labeled cells of the invention using the click reagents. The moiety should be coupled to a click reagent that can rapidly and selectively react (“click”) with its counterpart click reagent, i.e., the click reagent presented on the surface of a cell to be targeted, under mild conditions in aqueous solution. The mild conditions include neutral pH, aqueous solution and ambient temperature, with low reactant concentrations. In embodiments in which cells are labeled with a click reagent by recruitment and infiltration of a polymer scaffold device comprising the click reagent, as described herein, the click reagent presented on the surface of a cell to be targeted is also the click reagent present in the device. Exemplary click reagent pairs are well known to one of skill in the art and include, but are not limited to, azide and dibenzocyclooctyne (DBCO), tetrazine and transcyclooctene, and tetrazine and norbornene. Accordingly, a cell labeled with azide can be conjugated to a moiety that is coupled to DBCO. In other embodiments, a cell labeled with DBCO can be conjugated to a moiety that is coupled to azide. In other embodiments, a cell labeled with tetrazine can be conjugated to a moiety that is coupled to transcyclooctene or norbornene. In other embodiments, a cell labeled with transcyclooctene or norbornene can be conjugated to a moiety that is coupled to tetrazine. In embodiments of in which cells are labeled in vivo by recruitment to a device comprising one or more of the click reagents described herein, the moiety to be conjugated to a cell is coupled to a click reagent that can selectively react with the click reagent present in the device. For example, in embodiments in which a subject comprises a device comprising a click reagent that comprises azide, a moiety coupled to DBCO can be conjugated to click-labeled cells in the subject. Likewise, in another embodiment in which a subject comprises a device comprising a click reagent that comprises DBCO, a moiety coupled to azide can be conjugated to click-labeled cells in the subject. In another embodiment in which a subject comprises a device comprising a click reagent that comprises tetrazine, a moiety coupled to transcyclooctene or norbornene can be conjugated to click-labeled cells in the subject. In another embodiment in which a subject comprises a device comprising a click reagent that comprises transcyclooctene or norbornene, a moiety coupled to tetrazine can be conjugated to click-labeled cells in the subject.

In exemplary embodiments, the period of time sufficient for the click labeled cell to be targeted by the moiety is about 5 minutes, about 10 minutes, about 20 minutes, about 30 minutes, about 40 minutes, or more than about 40 minutes. Intermediaries and ranges between recited values are contemplated as part of the invention.

The moiety to be conjugated to the click labeled cells may be of various sizes. In some embodiments, the moiety may be a small protein or nucleic acid and have a molecular weight that is smaller than 10,000 Dalton with a hydrodynamic diameter between about 10 nm and about a thousand nanometer. In some embodiments, the moiety may have a molecular weight of about 1,000 Dalton, about 2,000 Dalton, about 3,000 Dalton, about 4,000 Dalton, about 5,000 Dalton, about 6,000 Dalton, about 7,000 Dalton, about 8,000 Dalton, about 9,000 Dalton, or about 10,000 Dalton. In certain embodiments, the moiety may have a hydrodynamic diameter of about 10 nm, about 20 nm, about 50 nm, about 100 nm, about 200 nm, about 300 nm, about 400 nm, about 500 nm, about 600 nm, about 700 nm, about 800 nm, about 900 nm, or about 1,000 nm. In some embodiment, the moiety may be of middle size, e.g., having a molecular weight between about 10,000 Dalton and about 1 million Dalton. In certain embodiments, the moiety has a molecular weight of about 10,000 Dalton, about 20,000 Dalton, about 50,000 Dalton, about 100,000 Dalton, about 200,000 Dalton, about 300,000 Dalton, about 400,000 Dalton, about 500,000 Dalton, about 600,000 Dalton, about 700,000 Dalton, about 800,000 Dalton, about 900,000 Dalton, or about 1 million Dalton. In certain embodiments, the moiety may also include large size nucleic acid, protein, or complex that contains multiple proteins and/or nucleic acids. Such a moiety may have a molecular weight in the range between about 1 million Dalton and about 1 billion Dalton and have a hydrodynamic diameter that is larger than 1 micrometer. In some embodiments, the moiety may have a molecular weight of about 1 million Dalton, about 2 million Dalton, about 5 million Dalton, about 10 million Dalton, about 20 million Dalton, about 50 million Dalton, about 100 million Dalton, about 200 million Dalton, about 300 million Dalton, about 400 million Dalton, about 500 million Dalton, about 600 million Dalton, about 700 million Dalton, about 800 million Dalton, about 900 million Dalton, or about 1 billion Dalton. In still some other embodiment, the moiety may have a hydrodynamic diameter of greater than about 1 μm, greater than about 2 μm, greater than about 5 μm, greater than about 10 μm, greater than about 20 μm, greater than about 30 μm, greater than about 40 μm, greater than about 50 μm, greater than about 60 μm, greater than about 70 μm, greater than about 80 μm, greater than about 90 μm, or greater than about 100 μm. The intermediaries and ranges between the recited values are contemplated as part of this invention.

Non-limiting examples of moieties that can be targeted to click-labeled cells include a small organic molecule, a small inorganic molecule; a saccharine; a monosaccharide; a disaccharide; a trisaccharide; an oligosaccharide; a polysaccharide; a peptide; a protein, a peptide analog, a peptide derivative; a peptidomimetic; an antibody (polyclonal or monoclonal); an antigen binding fragment of an antibody; a nucleic acid, e.g., an oligonucleotide, an antisense oligonucleotide, siRNAs, shRNAs, a ribozyme, an aptamer, microRNAs, pre-microRNAs, iRNAs, plasmid DNA (e.g. a condensed plasmid DNA), a modified RNA, and a nucleic acid analog or derivative. In some embodiments, the moiety is a cytokine, such as an anti-tumor cytokine. In some embodiments, the moiety is a therapeutic agent. In other embodiments, the moiety is a detection agent.

In some embodiments, the moieties targeted to a cell are attached, e.g., covalently linked, to a surface glycoprotein and remain on cell surface. In some embodiments, the moieties targeted to a cell are attached, e.g., covalently linked to a cell-surface glycoprotein.

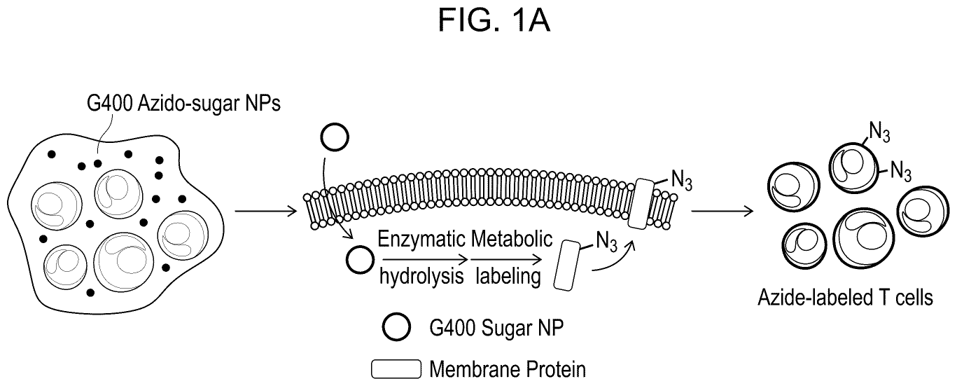

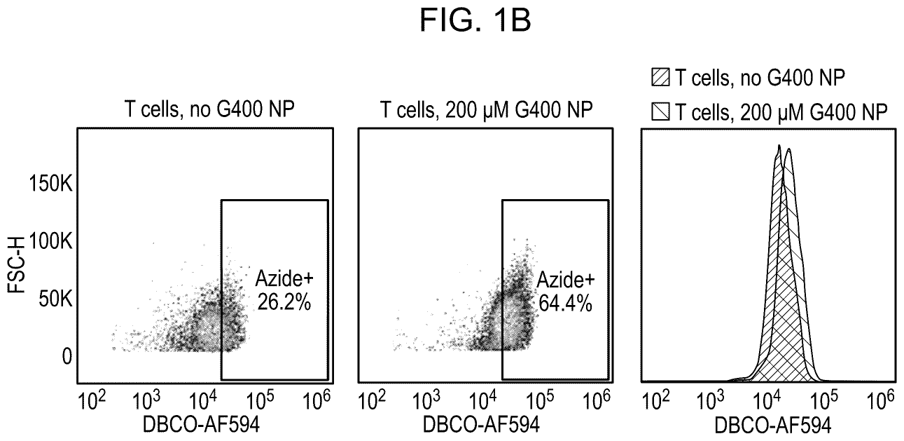

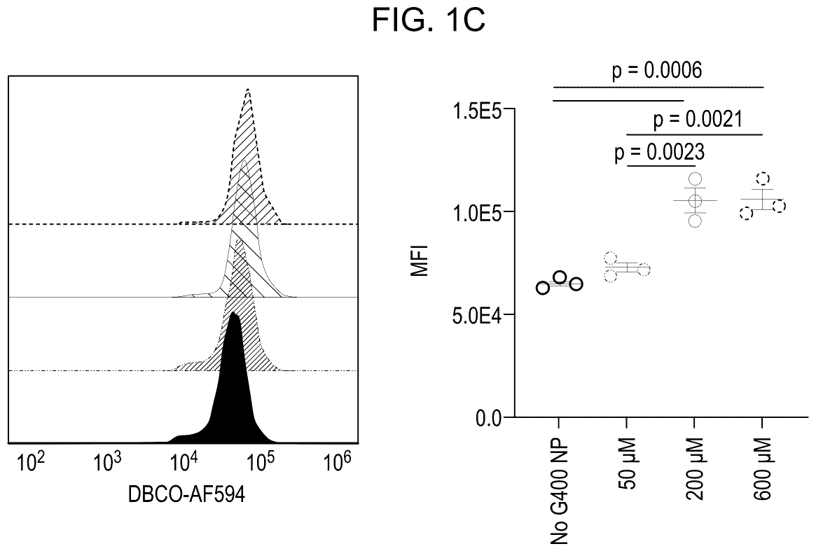

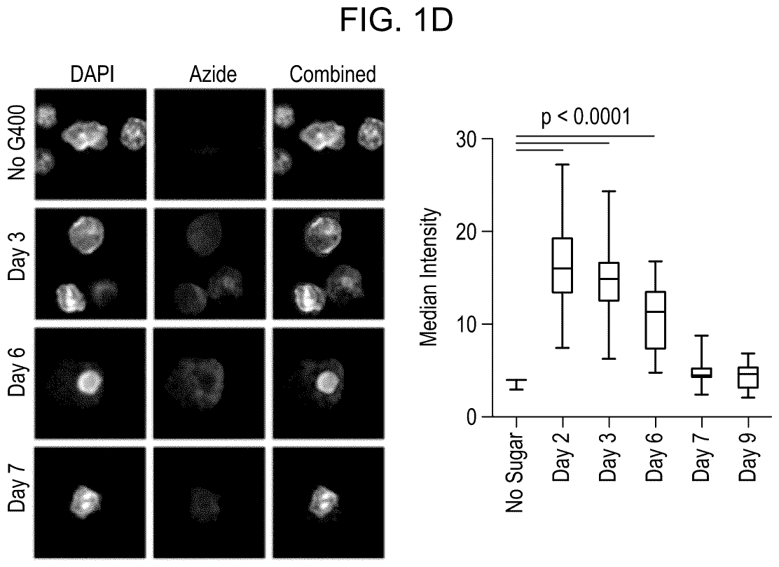

In some embodiments, without wishing to be bound by any theory, the moiety-glycoprotein protein complex formed through click chemistry is formed by unnatural azido-sugars, which have the ability to metabolically label cells. For example, unnatural azido-sugar nanoparticles, such as G400 NP comprising a polymer of azido sugar (n=400) derived from Ac₄ManAz, can be contacted with cells. After entering cells, unnatural azido-sugar nanoparticles, e.g., G400 NP, can degrade into sugar-azide via hydrolysis and enzymatic degradation, and be integrated into membrane glycoproteins to present azide groups on cell surfaces (FIG. 1A).

In other embodiments, without wishing to be bound by any theory, the moiety-glycoprotein protein complex formed through click chemistry is engulfed into the cell through endocytosis. After endocytosis, the moiety may be dissociated from the moiety-glycoprotein complex. In certain embodiments, the moiety is coupled to the click reagent through a linker, which can be cleaved within the cell selectively or specifically to facilitate the disassociation of the moiety from the glycoprotein. Exemplary linkers include, but are not limited to disulfide bond, hydrozone bond, or enzyme cleavable bond. The linker can be cleaved in endosome, facilitating the gene-editing moiety to be released from endosome. In some embodiments, the moiety retains its structural integrity, function, or activity after being targeted to a cell. A moiety retains its structural integrity when the moiety retains its intact structure or undergoes some structural changes but retains its function or activity. This allows for the delivery of an agent to a cell intracellularly so as to modify or manipulate the cell, e.g., physically, chemically, biochemically, physiologically, genetically, or epigenetically.

This strategy allows cells in vivo, ex vivo, or in vitro to be covalently coupled to virtually any agent. In some embodiments, an agent is targeted to a cell in vitro, comprising contacting a cell coupled to a click reagent with an agent coupled to a counterpart click reagent. The cell may be cultured in vitro. In general, the moiety coupled to a click reagent may be added to a cell culture medium to contact the cell coupled to a counterpart click reagent.

In some embodiments, a cell may be contacted in vivo, ex vivo, or in vitro with at least one, at least two, at least three, at least four, at least 5, at least 6, at least 7, at least 8, at least 9, or at least 10, or more agents coupled to a click reagent, optionally, a plurality of times.

In some embodiments, a plurality of cells may be contacted in vivo, ex vivo, or in vitro with at least one, at least two, at least three, at least four, at least 5, at least 6, at least 7, at least 8, at least 9, or at least 10, or more agents coupled to a click reagent, optionally, a plurality of times.

Accordingly, the present invention provides a cell comprising an agent/moiety attached to a glycoprotein through click-reaction. In certain embodiments, the agent-glycoprotein complex may be present intracellularly. In some embodiments, a cell may comprise at least one, at least two, at least three, at least four, at least 5, at least 6, at least 7, at least 8, at least 9, or at least 10, or more agent/moieties attached to a glycoprotein through click-reaction, optionally, wherein each agent/moiety is the same, or wherein each agent/moiety is different.

In some embodiments, the present invention provides a plurality of cells (e.g., a population of cells) comprising an agent/moiety attached to a glycoprotein through click-reaction. In certain embodiments, the agent-glycoprotein complex may be present intracellularly. In some embodiments, a plurality of cells (e.g., a population of cells) may comprise at least one, at least two, at least three, at least four, at least 5, at least 6, at least 7, at least 8, at least 9, or at least 10, or more agent/moieties attached to a glycoprotein through click-reaction, optionally, wherein each agent/moiety is the same, or wherein each agent/moiety is different.

In an exemplary embodiment, the click-coupled moieties are targeted to click-labeled immune cells, e.g., click-labeled T cells.

In one embodiment, the click-coupled moiety is a protein, a peptide, a nucleic acid, or a small molecule. In an exemplary embodiment, the click-coupled moiety is a protein or a peptide, or a polynucleotide encoding the protein or peptide. When the moiety refers to a protein or a peptide, it is contemplated that the polynucleotide encoding such protein or a peptide is also a click-coupled moiety. Non-limiting exemplary protein or peptide includes, but is not limited to, transcriptional factor, growth factor, cytokine, antibody, and/or gene editing molecules. The protein or peptide may be a fusion protein that comprises a reporter protein or peptide, e.g., GFP, to facilitate the screening and/or selection of cells that are targeted by the click-coupled moiety.

In some embodiments, the click-coupled moiety is a nucleic acid. The nucleic acid may be synthesized to incorporate a reactive group, such as an amine or thiol group, to be conjugated to a click reagent, e.g., DBCO by reacting with DBCO-NHS or DBCO-maleimide.

Using this approach, cells can be covalently coupled to a detectable label. For example, click-labeled cells can be contacted with a detectable label coupled to a second click reagent, which selectively reacts with the click reagent on the click-labeled cells. In embodiments where cells are covalently coupled to a detectable label in vivo, this can be accomplished by administering the detectable label coupled to the second click reagent to a subject. The detectable label can be a fluorescent label. Exemplary fluorescent labels include, but are not limited to, Alexa Fluor (e.g., Alexa Fluor 405, Alexa Fluor 488, Alexa Fluor 700, Alexa Fluor 750, etc.), GFP, FITC, CFSE, DyLight 488, phycoerythrin (PE), propidium iodide (PI), PerCP, Cy5, Cy5.5, Cy7, APC-eFluor 780, Draq-5, APC, amine aqua, pacific orange, pacific blue, DAPI, eFluor 450, eFluor 605, eFluor 625, and eFluor 650. In other embodiments, the detectable label can be a radiolabel. Exemplary radiolabels include, but are not limited to, ³H, ¹⁴C, ¹³N, ¹⁵O, ¹⁸F, ³²P, ³⁵S, ^{99m}Tc, ¹²³I, ¹²⁵I, and ⁶⁷Ga.

In some embodiments, cells can be covalently coupled to a cytokine. For example, click-labeled cells can be contacted with a cytokine coupled to a second click reagent, which selectively reacts with the click reagent on the click-labeled cells. In embodiments where cells are covalently coupled to a cytokine in vivo, this can be accomplished by administering the cytokine coupled to the second click reagent to the subject. Examples of cytokines include, but are not limited to, interleukins, interferons, chemokines, tumor necrosis factors, and colony stimulating factors of immune cell precursors. In some embodiments, interleukins can be human interleukins including IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, and IL-35. In some embodiments, chemokines include, but are not limited to, CCL family, CXCL family, CX3CL family and XCL family. The term “inflammatory cytokine” as used herein generally includes, without limitation, a cytokine that stimulates an inflammatory response. Examples of inflammatory cytokines include, without limitation, IFN-γ, IL-1, and TNF-α.

Exemplary cytokines also include, but are not limited to, interleukin-2 (IL-2), interleukin-7 (IL-7), interleukin-12 (IL-12), interleukin-15 (IL-15), interleukin-18 (IL-18), interleukin-21 (IL-21), interleukin-27 (IL-27), interferon-α (IFN-α), interferon-γ (IFN-γ)granulocyte macrophage-colony stimulating factor (GM-CSF), Fms-like tyrosine kinase receptor 3 ligand (Flt3-L), and tumour necrosis factor α (TNF-α). In some embodiments, the cytokine includes interleukin 15 (IL-15), interleukin 13 (IL-10), IL-2, interleukin 12 (IL-12), interleukin 15 (IL-15), interleukin 18 (IL-18), tumor necrosis factor alpha (TNFα), interferon gamma (IFNγ), granulocyte-macrophage colony-stimulating factor (GM-CSF), or a combination thereof. In one embodiment, the cytokine receptor is IL-15.

In some embodiments, (i) the cytokine is selected from the group consisting of interleukins, interferons, chemokines, tumor necrosis factors, and colony stimulating factors of immune cell precursors; (ii) the cytokine is an interleukin selected from the group consisting of IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, and IL-35; (iii) the cytokine is a chemokine selected from the group consisting of CCL family, CXCL family, CX3CL family, and XCL family chemokines; (iv) the cytokine is an inflammatory cytokine selected from the group consisting of IFN-γ, IL-1, and TNF-α; (v) the cytokine is selected from the group consisting of interleukin-2 (IL-2), interleukin-7 (IL-7), interleukin-10 (IL-10), interleukin-12 (IL-12), interleukin-15 (IL-15), IL-15/IL-15Rα, interleukin-18 (IL-18), interleukin-21 (IL-21), interleukin-27 (IL-27), interferon-α (IFN-α), interferon-γ (IFN-γ), granulocyte macrophage-colony stimulating factor (GM-CSF), Fms-like tyrosine kinase receptor 3 ligand (Flt3-L), tumour necrosis factor α (TNF-α), and combinations thereof; and/or (vi) the cytokine is an anti-tumor cytokine.

Accordingly, immune cells, e.g., T cells, may be manipulated using click chemistry.

In some embodiments, the present invention provides a method to modulate, e.g., enhance, immune cell activity, e.g., anti-tumor activity. The method comprises contacting a cell coupled to a click reagent with a cytokine coupled to a counterpart click reagent.

In certain embodiments, the cells are manipulated in vitro or ex vivo. The targeting of cells using click chemistry pairs may be combined with other treatment or manipulation of the cells. For example, cells may be screened, selected, expanded and/or differentiated after a click-couple moiety is targeted to the cells.

Advantageously, the click reagent coupled moiety can be specifically targeted to a cell comprising surface glycoprotein coupled with a counterpart click reagent, thereby increasing the specificity of the targeting of the moiety. This is particularly useful for targeting a cell that is labeled with a click reagent in vivo. For example, a cell may be metabolically labeled with a click reagent in vitro and administered to a subject. A moiety coupled to a counterpart click reagent may be administered to a subject separately. The metabolically labeled cell can specifically be linked to the moiety coupled to a click reagent through click chemistry in vivo. The administration of the click reagent-coupled moiety may be prior to, concurrently with, or after the administration of the cell labeled with a counterpart click reagent.

For in vivo targeting an agent to an immune cell, a moiety coupled to a click reagent can be administered to a subject, e.g., a subject comprising click-coupled cells, by any suitable method. A compound or composition described herein can be administered by any appropriate route known in the art including, but not limited to, oral or parenteral routes, including intravenous, intramuscular, subcutaneous, transdermal, airway (aerosol), pulmonary, nasal, rectal, and topical (including buccal and sublingual) administration.

Exemplary modes of administration include, but are not limited to, injection, infusion, instillation, inhalation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In preferred embodiments, the compositions are administered by injection, e.g., subcutaneous injection or intratumoral injection, or by intravenous infusion.

Administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the compounds are formulated into ointments, salves, gels, or creams as generally known in the art.

In some embodiments, the administration is by subcutaneous injection.

In one embodiment, the site of administration is at or near the site of a tumor in a subject. For example, a moiety coupled to a click reagent can be administered within 5 cm of a tumor, e.g., within 4 cm, within 3 cm, within 2 cm, or within 1 cm of a tumor in the subject. In other embodiments, a moiety coupled to a click reagent can be administered within 10 mm of tumor, e.g., within 9 mm, within 8 mm, within 7 mm, within 6 mm, within 5 mm, within 4 mm, within 3 mm, within 2 mm, or within 1 mm of a tumor in a subject. In other embodiments, a moiety coupled to a click reagent can be administered intratumorally.

In another embodiment, the site of administration is distal from the site of a tumor in a subject. For example, a moiety coupled to a click reagent can be administered more than 5 cm from the site of a tumor.

In some embodiments, the administration is by intravenous injection.

Determination of an effective amount is well within the capability of those skilled in the art. Generally, the actual effective amount can vary with the specific compound, the use or application technique, the desired effect, the duration of the effect and side effects, the subject's history, age, condition, sex, as well as the severity and type of the medical condition in the subject, and administration of other pharmaceutically active agents. Accordingly, an effective dose of compound described herein is an amount sufficient to produce at least some desired therapeutic effect in a subject.

The term “therapeutically effective amount”, as used herein, means that amount of a compound, material, or composition comprising a compound described herein which is effective for producing some desired therapeutic effect in at least a sub-population of cells in a subject at a reasonable benefit/risk ratio applicable to any medical treatment.

In one embodiment, the site of administration is at or near the site of a polymer scaffold device of the invention. For example, a moiety coupled to a click reagent can be administered within 5 cm of the device, e.g., within 4 cm, within 3 cm, within 2 cm, or within 1 cm of the device in the subject. In other embodiments, a moiety coupled to a click reagent can be administered within 10 mm the device, e.g., within 9 mm, within 8 mm, within 7 mm, within 6 mm, within 5 mm, within 4 mm, within 3 mm, within 2 mm, or within 1 mm of the device in a subject. In other embodiments, a moiety coupled to a click reagent is administered at the site of the device in a subject.

In another embodiment, the site of administration is distal from the site of the device in a subject. For example, a moiety coupled to a click reagent can be administered more than 5 cm from the site of the device.

In one embodiment, the amount is a therapeutically effective amount.

Determination of an effective amount is well within the capability of those skilled in the art. Generally, the actual effective amount can vary with the specific compound, the use or application technique, the desired effect, the duration of the effect and side effects, the subject's history, age, condition, sex, as well as the severity and type of the medical condition in the subject, and administration of other pharmaceutically active agents. Accordingly, an effective dose of compound described herein is an amount sufficient to produce at least some desired therapeutic effect in a subject. In one embodiment, the amount is a therapeutically effective amount. In another embodiment, the amount is an immunogenic amount.

The term “therapeutically effective amount”, as used herein, means that amount of a compound, material, or composition comprising a compound described herein which is effective for producing some desired therapeutic effect in at least a sub-population of cells in a subject at a reasonable benefit/risk ratio applicable to any medical treatment. Thus, “therapeutically effective amount” means that amount which, when administered to a subject for treating a disease, is sufficient to effect such treatment for the disease.

The term “immunogenic amount” of an antigen and/or adjuvant refers to an amount of antigen and/or adjuvant sufficient to stimulate a useful immune response. The amount of antigen and/or adjuvant necessary to provide an immunogenic amount is readily determined by one of ordinary skill in the art, e.g., by preparing a series of vaccines of the invention with varying concentrations of antigen and/or adjuvant, administering the vaccines to suitable laboratory animals (e.g., mice, rats, guinea pigs, etc.), and assaying the resulting immune response by measuring serum antibody titer, antigen-induced swelling in the skin, and the like.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of use or administration utilized.

In one embodiment, the dosage is a weight-based dose. In exemplary embodiments, the weight-based dose is 0.001-100 mg/kg. For example, in some embodiments, the dosage is 0.001-0.1 mg/kg. In other embodiments, the dosage is 0.01-1 mg/kg. In other embodiments, the dosage is 0.1-10 mg/kg. In other embodiments, the dosage is 1-100 mg/kg. In other embodiments, the dosage is about 1 mg/kg, 2 mg/kg, 5 mg/kg, 10 mg/kg, 20 mg/kg, 30 mg/kg, 40 mg/kg, 50 mg/kg, 60 mg/kg, 70 mg/kg, 80 mg/kg, 90 mg/kg, or 100 mg/kg.

The effective dose can be estimated initially from cell culture assays. A dose can be formulated in animal models to achieve a circulating plasma concentration range that includes the IC₅₀ (i.e., the concentration of the therapeutic which achieves a half-maximal inhibition of symptoms) as determined in cell culture. Levels in plasma can be measured, for example, by high performance liquid chromatography. The effects of any particular dosage can be monitored by a suitable bioassay.

In embodiments in which cells are labeled with a click reagent in vivo by recruitment of cells to a polymer scaffold device of the invention comprising the click reagent, a moiety for conjugation to the cell is preferably administered to the subject after a period of time sufficient for labeling of cells in the subject with the click reagent present in the device. The average time for cells in a subject to become labeled with a click reagent following administration of the device can be determined empirically, for example, by detecting the presence of click-labeled cells in a test subject using a click reagent coupled to a detectable label. In exemplary embodiments, the moiety for conjugation to click-labeled cells is administered to a subject at least 2 days, at least 3 days, at least 4 days, at least 5 days, at least 6 days, at least 7 days, at 8 days, at least 9 days, at least 10 days, at least 12 days, at least 14 days, or at least 21 days after administration of the device to the subject. For example, in some embodiments, the moiety is administered about 2-21 days after administration of the device. In some embodiments, the moiety is administered about 4-21 days after administration of the device. In some embodiments, the moiety is administered about 4-14 days after administration of the device. In some embodiments, the moiety is administered about 4-10 days after administration of the device. In some embodiments, the moiety is administered about 6-10 days after administration of the device.

### VI. Pharmaceutical Compositions

For administration to a subject, the unnatural sugars, polymers, nanoparticles, devices, scaffolds, hydrogels, agents coupled to click chemistry reagents, and cells described herein can be provided as pharmaceutically acceptable (e.g., sterile) compositions. Accordingly, in one aspect, the invention provides a pharmaceutical composition comprising an unnatural sugar, a polymer, and/or a nanoparticle comprising a click reagent. In another aspect, the invention provides a pharmaceutical composition comprising a device that comprises polymer scaffold comprising a click reagent. In one embodiment, the polymer scaffold is a hydrogel.

These pharmaceutically acceptable compositions can be formulated together with one or more pharmaceutically acceptable carriers (additives) and/or diluents. As described in detail below, the pharmaceutical compositions of the present disclosure can be specifically formulated for administration in solid or liquid form, including those adapted for the following: (1) parenteral administration, for example, by subcutaneous, intramuscular, intravenous (e.g., bolus or infusion) or epidural injection as, for example, a sterile solution or suspension, or sustained-release formulation; (2) oral administration, for example, drenches (aqueous or non-aqueous solutions or suspensions), lozenges, dragees, capsules, pills, tablets (e.g., those targeted for buccal, sublingual, and/or systemic absorption), boluses, powders, granules, pastes for application to the tongue; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) transmucosally; or (9) nasally. Additionally, compounds can be implanted into a patient or injected using a drug delivery system. See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. No. 3,773,919; and U.S. Pat. No. 35 3,270,960, content of all of which is herein incorporated by reference.

As used herein, the term “pharmaceutically acceptable” or “pharmacologically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. Moreover, for animal (e.g., human) administration, it will be understood that compositions should meet sterility, pyrogenicity, general safety and purity standards as required by FDA Office of Biological Standards.

As used herein, the term “pharmaceutically acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, disintegrating agents, binders, sweetening agents, flavoring agents, perfuming agents, protease inhibitors, plasticizers, emulsifiers, stabilizing agents, viscosity increasing agents, film forming agents, solubilizing agents, surfactants, preservative and antioxidants can also be present in the formulation. The terms such as “excipient”, “carrier”, “pharmaceutically acceptable carrier” or the like are used interchangeably herein.

The pharmaceutical compositions of the invention comprising a click reagent can be delivered to an in vivo locus in a subject. Exemplary in vivo loci include, but are not limited to site of a wound, trauma or disease. The composition can be delivered to the in vivo locus by, for example, implanting the compositions into a subject. The composition can optionally include one or more additives. Additives can include, but are not limited to, resolving (biodegradable) polymers, mannitol, starch sugar, inosite, sorbitol, glucose, lactose, saccharose, sodium chloride, calcium chloride, amino acids, magnesium chloride, citric acid, acetic acid, hydroxyl-butanedioic acid, phosphoric acid, glucuronic acid, gluconic acid, poly-sorbitol, sodium acetate, sodium citrate, sodium phosphate, zinc stearate, aluminium stearate, magnesium stearate, sodium carbonate, sodium bicarbonate, sodium hydroxide, polyvinylpyrolidones, polyethylene glycols, carboxymethyl celluloses, methyl celluloses, starch or their mixtures.

### VII. Kits

Any of the compositions described herein may be comprised in a kit. In a non-limiting example, the kit comprises a click functionalized polysaccharide polymer which is a product of radical-catalyzed polymerization. In certain embodiments, the kit includes unnatural sugars and/or nanoparticles for labeling cells with a click reagent comprising the click functionalized polysaccharide polymer. In some embodiments, the kit includes the device and/or scaffold described elsewhere herein. In a non-limiting example, the kit includes a device including a polymer scaffold, a click reagent, and a chemoattractant for immune cells. In certain embodiments, the kit comprises a click functionalized polysaccharide polymer which is a product of radical-catalyzed polymerization and a second click chemistry reagent coupled to an agent targeted to the immune cell, wherein the second click chemistry reagent can selectively react with the click reagent present in the functionalized polysaccharide polymer. In some embodiments, the kit includes unnatural sugars and/or nanoparticles for labeling cells with a click reagent comprising the click functionalized polysaccharide polymer and a second click chemistry reagent coupled to an agent targeted to the immune cell, wherein the second click chemistry reagent can selectively react with the click reagent present in the unnatural sugar and/or nanoparticle. In certain embodiments, the kits includes a device comprising polymer scaffold, a click reagent, and a chemoattractant for immune cells, and a second click chemistry reagent coupled to an agent targeted to the immune cell, wherein the second click chemistry reagent can selectively react with the click reagent present in the device.

The kit may further include reagents or instructions for in vivo labeling a cell in a subject and/or in vitro labeling a cell with a click chemistry reagent described elsewhere herein. It may also include one or more buffers. Other kits of the invention may include components for assays to detect the labeling of the cell. In certain embodiments, the kits of the invention comprise the reagents for detecting a detectable label that is targeted to a cell.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit (labeling reagent and label may be packaged together), the kit also will generally contain a second, third or other additional container into which the additional components may be separately placed. The kits may also comprise a second container means for containing a sterile, pharmaceutically acceptable buffer and/or other diluent. However, various combinations of components may be comprised in a vial. The kits of the present invention also will typically include a means for containing the compositions of the invention, e.g., the click functionalized polysaccharide polymer, and any other reagent containers in close confinement for commercial sale.

When the components of the kit are provided in one and/or more liquid solutions, the liquid solution is an aqueous solution, with a sterile aqueous solution being particularly preferred. However, the components of the kit may be provided as dried powder(s). When reagents and/or components are provided as a dry powder, the powder can be reconstituted by the addition of a suitable solvent. It is envisioned that the solvent may also be provided in another container means.

The present invention is further illustrated by the following examples, which should not be construed as limiting. The entire contents of all of the references cited throughout this application are hereby expressly incorporated herein by reference.

### EXAMPLES

### Example 1: Use of Unnatural Sugar Nanoparticles to Specifically Label T Cells

Metabolically Labeled T Cells does not Affect T Cell Functions and Phenotype

This experiment explored the ability of unnatural azido-sugars to metabolically label T cells. G400 nanoparticles (NPs), a polymer of azido sugar (n=400) derived from Ac₄ManAz previously developed in the lab, was added to T cell cultures (cite). After entering T cells, G400 NP degraded into sugar-azide via hydrolysis and enzymatic degradation, and was eventually integrated into membrane glycoproteins to present azide groups on T cell surfaces (FIG. 1A). To verify T cell labeling with G400, various concentrations of G400 were added to freshly isolated mouse T cells in vitro. After 72 hours, metabolic labeling of T cells were detected as reflected by the increased percentage of azide positive cells (FIG. 1B). Furthermore, metabolic labeling of T cells was dose dependent as reflected by cell-surface azide intensity, with saturation reached at 200 μM of G400 NPs (FIG. 1C). This experiment next looked at the persistence and durability of azide labeling on T cell surfaces as T cells grow and proliferate. Freshly isolated mouse T cells were treated with 200 μM G400 NPs for 3 days and kept in culture for another week. T cells were stained for azide at various time points during and after G400 NP treatment (FIG. 1D). T cells were successfully azido-labeled by day 2, and after G400 NP removal, T cells continue to exhibit azide signal, demonstrating that labeling by G400 NP is persistent at a considerable level for at least 4 days (FIG. 1D). Azide signal decrease over time was expected given the high proliferation rate of T cells. It was also noticed that there was detectable azide signal inside T cells as well, indicating that certain azido-labeled glycoproteins may be endocytosed.

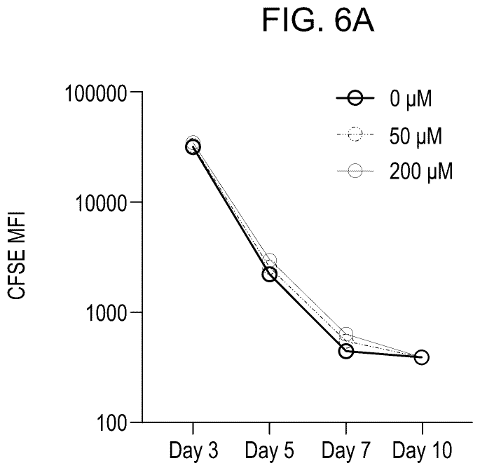

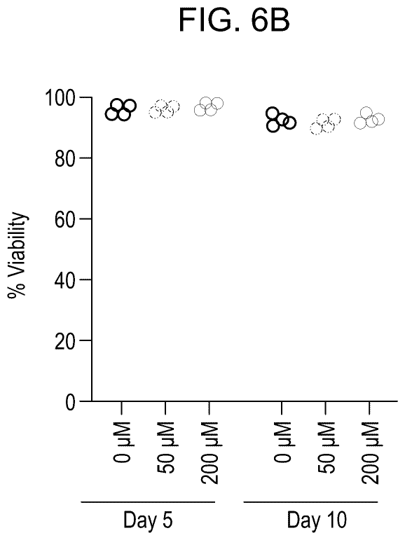

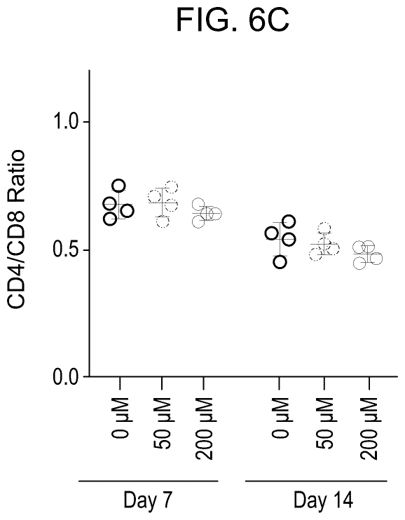

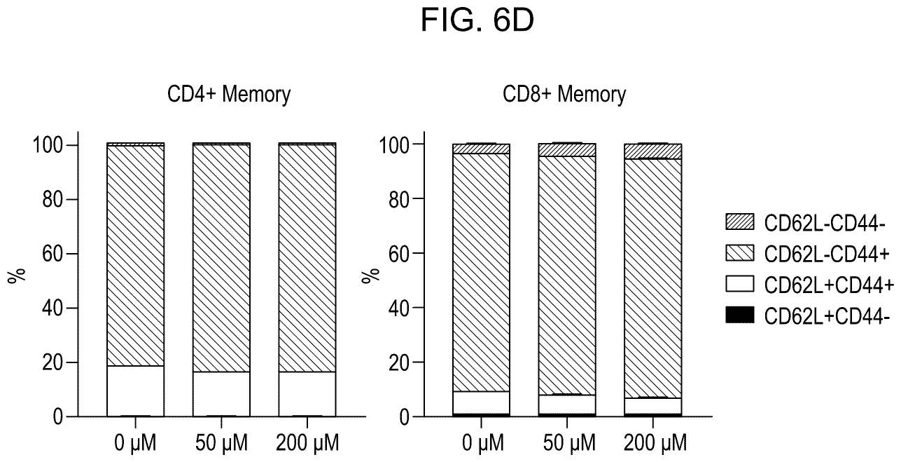

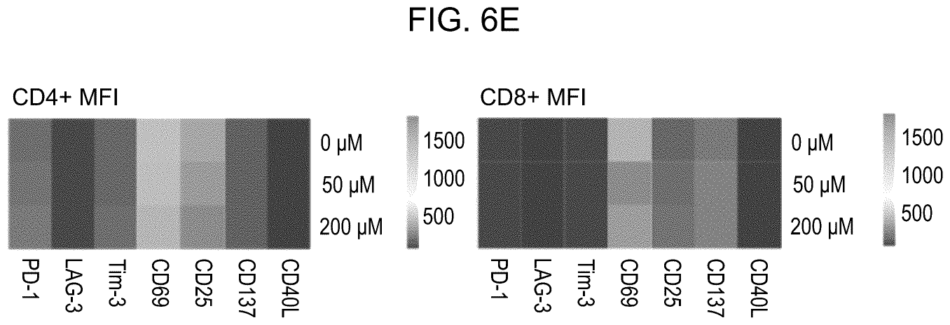

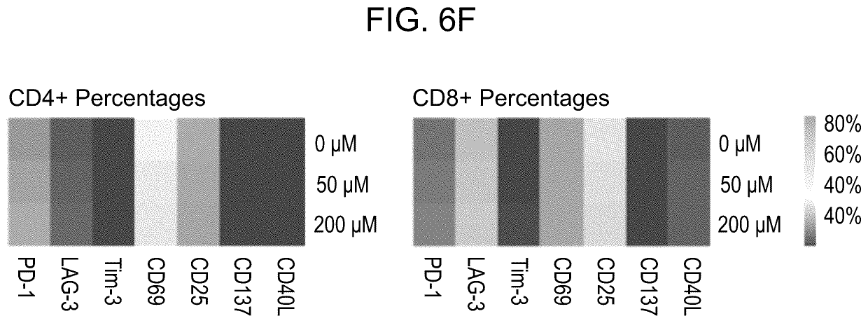

Next it was investigated whether azido-labeling with G400 NPs would significantly alter T cell functions and phenotypes. T cells were treated with various concentrations of G400 NP for two weeks, and their proliferation, viability, memory, activation, and exhaustion phenotypes were closely monitored with flow cytometry. High concentrations of G400 NP treatment didn't alter T cell proliferation and survival abilities (FIGS. 6A-6B), with only small changes in certain phenotypes observed (FIGS. 6D-6F). Overall, G400 NP treatment did not affect T cell's ability to proliferate and survive, nor did it induce phenotypical changes that may alter T cell functions. Therefore, we used 200 μM G400 NPs for subsequent studies for maximum azido-labeling saturation.

Conjugating Cytokines onto Azido-Labeled T Cells Via Click Chemistry

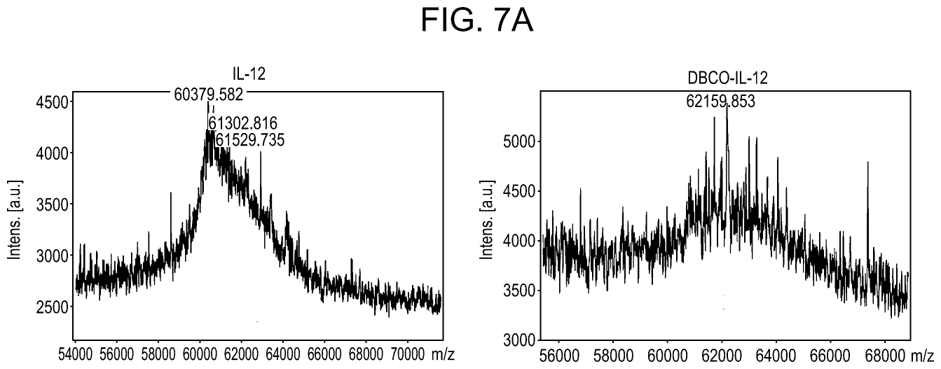

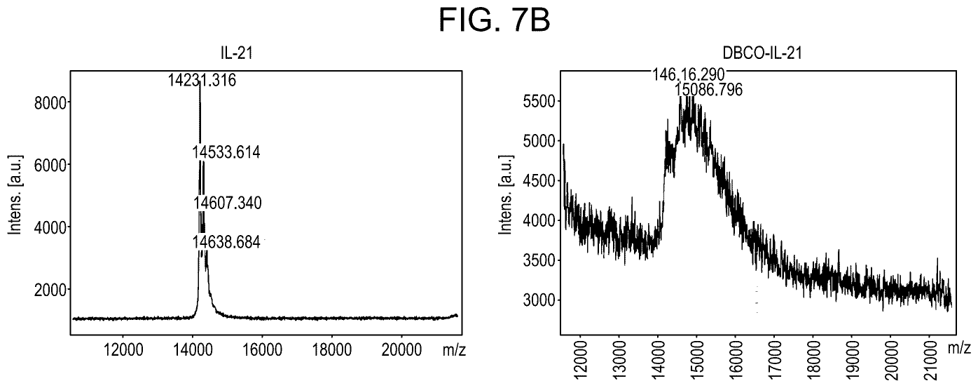

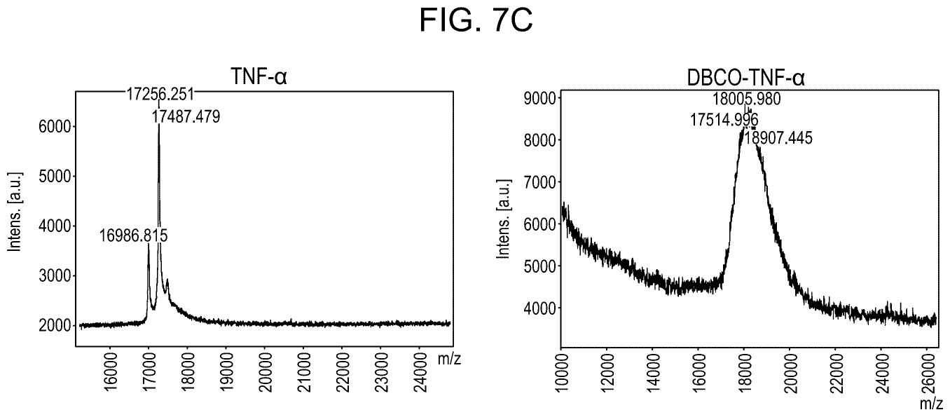

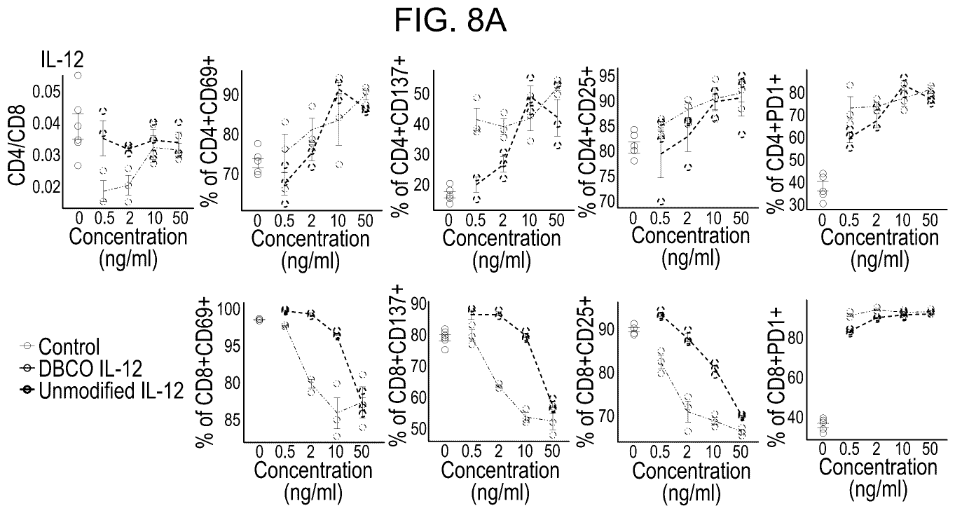

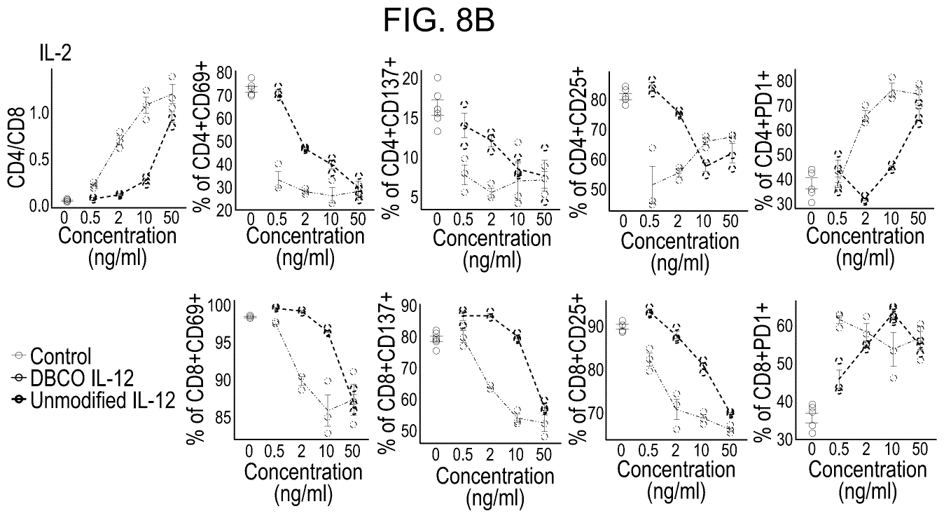

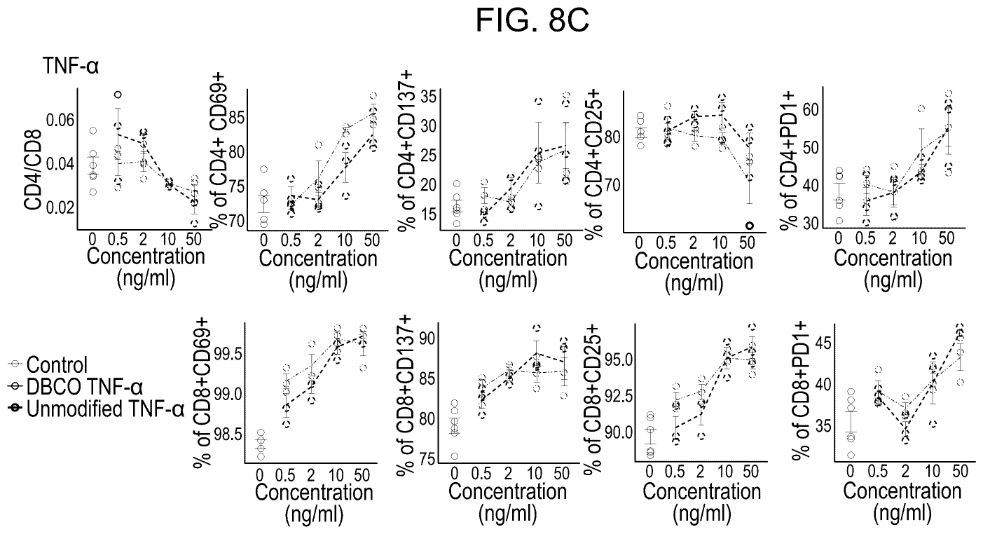

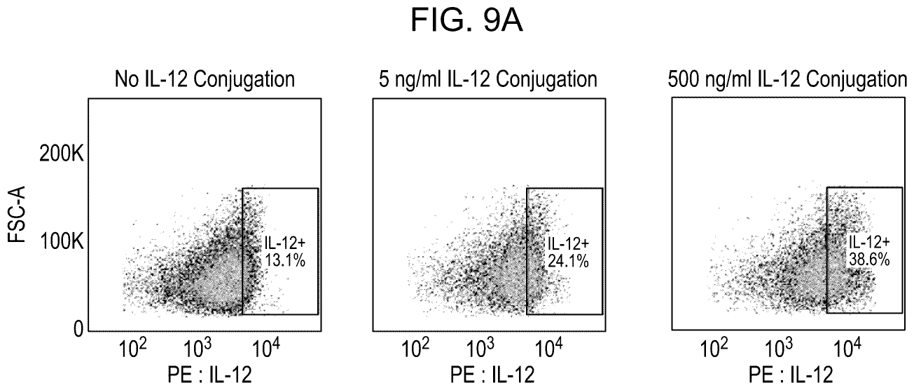

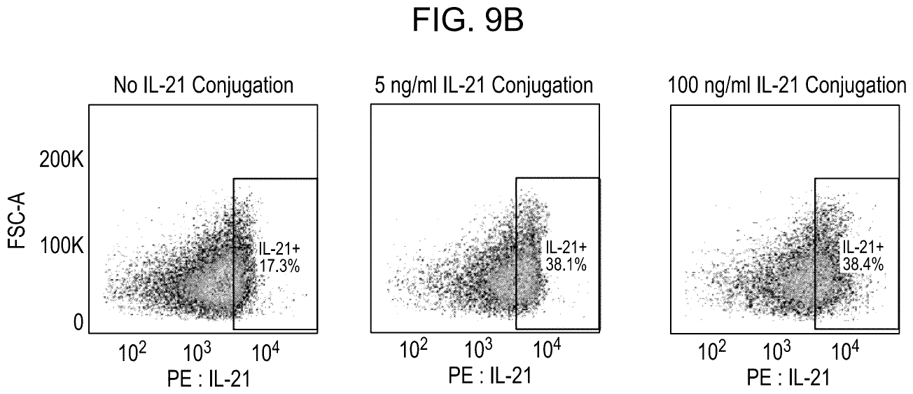

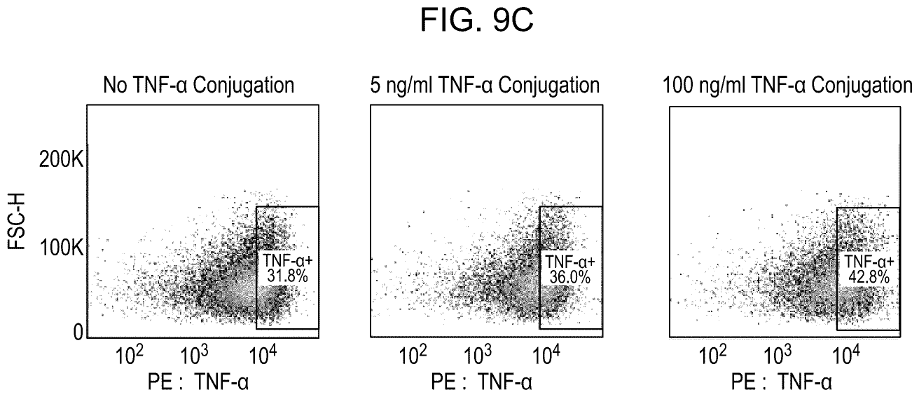

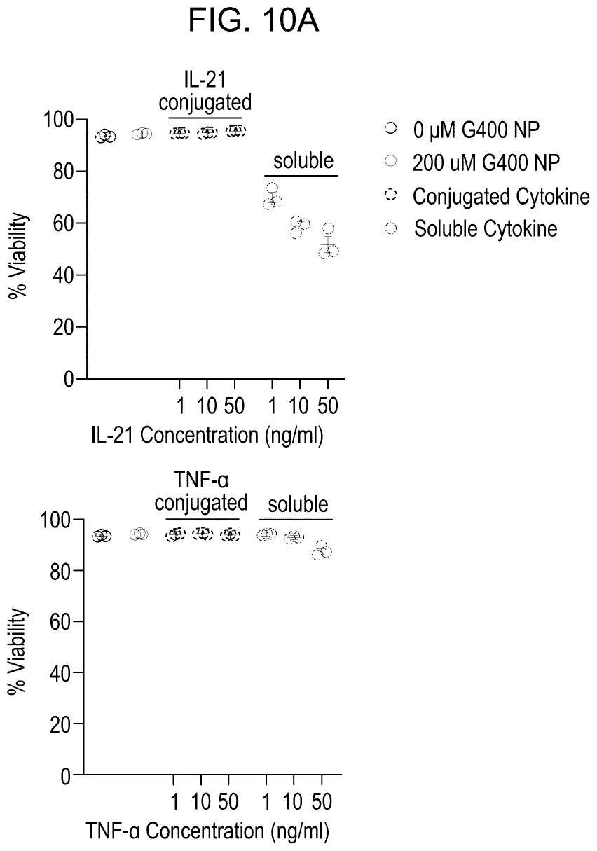

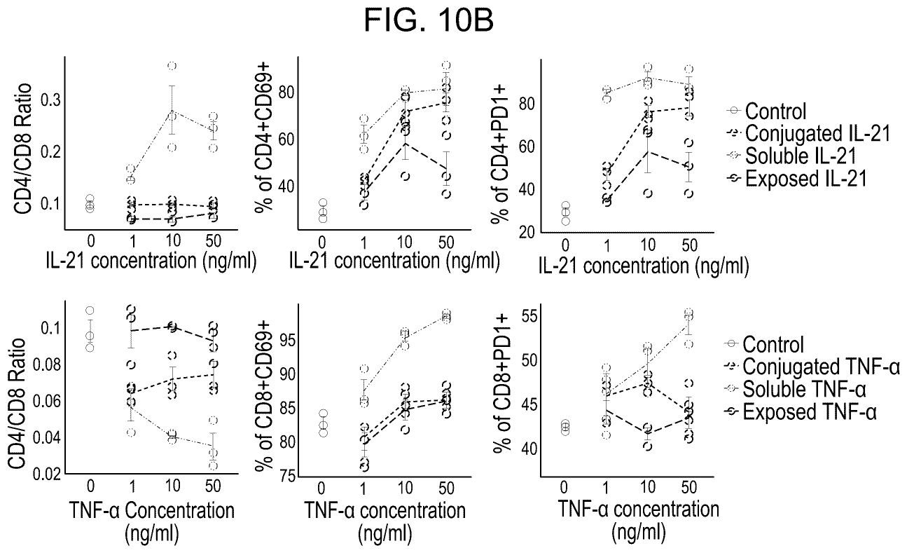

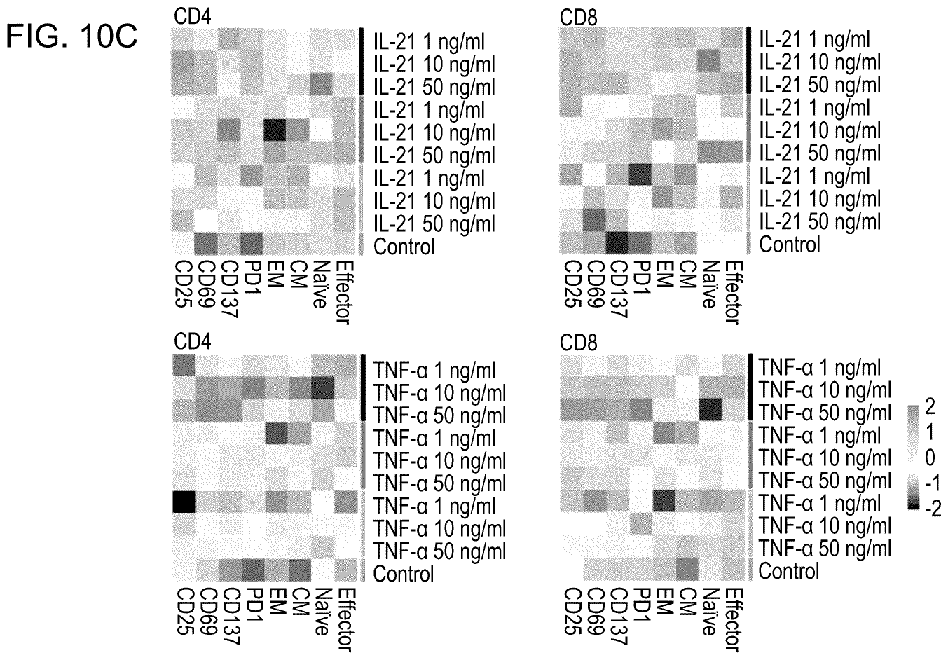

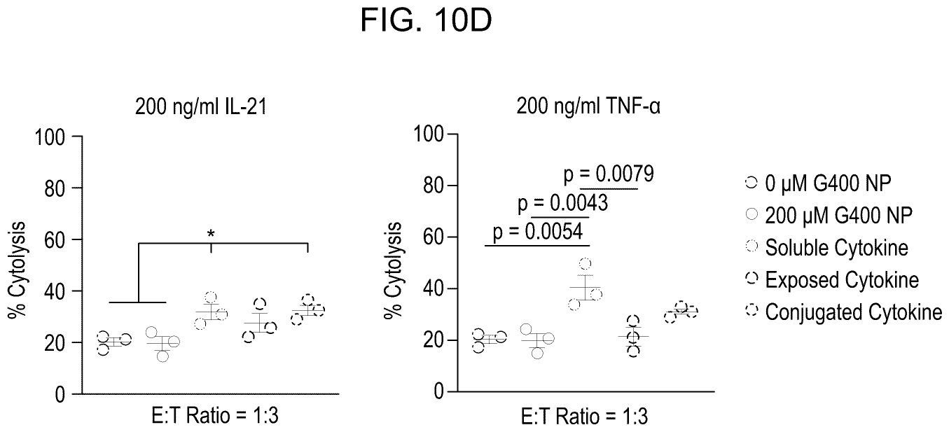

This experiment next looked at the ability of these azido-labeled T cells to mediate targeted delivery of DBCO-modified agents, and whether these agents could bias azido-labeled T cells towards the desire phenotype (FIG. 2A). We tested the ability for various anti-tumor cytokines, including IL-12, IL-21, and TNF-α, to conjugate onto T cell surfaces via click chemistry. Anti-tumor cytokines were first modified with DBCO-sulfo-NHS to link DBCO groups onto the primary amines of the protein, and the modification was confirmed via MALDI-TOF (FIGS. 7A-7C). The bioreactivity of DBCO-modified cytokines were then compared with unmodified cytokines on T cells, and were, not surprisingly, slightly less potent than their unmodified counterparts (FIGS. 8A-8C), possibly due to chemical modification happening at the interaction cite. Additionally, we found that bioreactivity is inversely correlated with the degree of modification: the more DBCO groups that were linked onto the cytokine, the lower bioreactivity of the DBCO-modified cytokine. It was found that the use of 2-3 DBCO molecules per protein for the conjugation preserved its bioreactivity. Next, this experiment tested the ability for DBCO-cytokines to be conjugated onto T cells and direct differentiation. DBCO-modified cytokines were conjugated onto T cells via click reaction for 30 minutes at 4° C., and T cells are subsequently washed to remove any excessive, non-conjugated cytokine. It was found that cytokine conjugation via click chemistry was dose-dependent, with higher concentrations of DBCO-cytokines resulting in higher percentage of cell-surface cytokine-labeled T cells (FIGS. 9A-9C). Most anti-tumor cytokines, when presented to T cells in large amounts in the native soluble form, exhibit high toxicity and prevents T cells from proliferating and surviving. However, surprisingly, when these cytokines were conjugated onto the T cells, they exhibit less cytotoxicity and inhibition on T cell proliferation (FIG. 2B, FIG. 10A). In fact, T cells conjugated with high dose of IL-12, IL-21, and TNF-α demonstrated similar viability to untreated T cells, and much better viability and proliferation compared with T cells receiving soluble cytokine in their culture. We then examined whether conjugating cytokines onto T cell surfaces could direct T cell differentiation by profiling T cell phenotype via flow cytometry 7 days after cytokine conjugation. We showed that conjugating cytokines onto T cell surfaces could direct T cell differentiation in the same direction as their soluble counterparts do, as demonstrated by CD4/CD8 ratio, memory, activation, and exhaustion markers in a dose-dependent manner (FIGS. 2C-2D, FIGS. 10B-10C). This effect is caused by cytokines being conjugated onto T cells for an extended period of time, since exposing T cells to unmodified cytokines only for the time needed for the conjugation reaction (“Exposed IL-12”) doesn't bring significant effect to T cell phenotypes. This effect on T cell phenotype is translated into T cell function, as reflected by the cytotoxicity of Pmel-1 T cells against B16-F10 melanoma tumor cells, with T cells conjugated with anti-tumor cytokines killing almost as good as T cells receiving soluble cytokines (FIG. 2E, FIG. 8D). While the conjugation works for all three cytokines we tested, we did notice that certain cytokines perform better than others at differentiating T cells after DBCO-modification and conjugation. Therefore, we picked IL-12, which performed the best in terms of its ability to direct T cell differentiation and obtain the desired phenotype, for subsequent studies. We further quantified the amount of DBCO-IL-12 loaded onto T cells with ELISA. The amount of loading was positively correlated with the concentration of cytokines during the click reaction. With 200 ng/ml of IL-12, a high concentration for soluble treatments, we were able to load ˜210 ng per 1 million cells. This is a relatively high dose compared what has been previously used in human clinical trials but much lower compared with other mouse studies that focused on cytokine delivery.

### Cytokine Conjugation Promoted T Cell Persistence and Effector Differentiation In Vivo

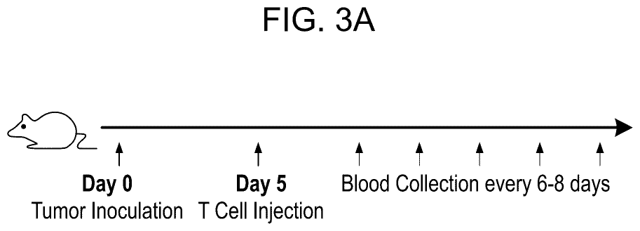

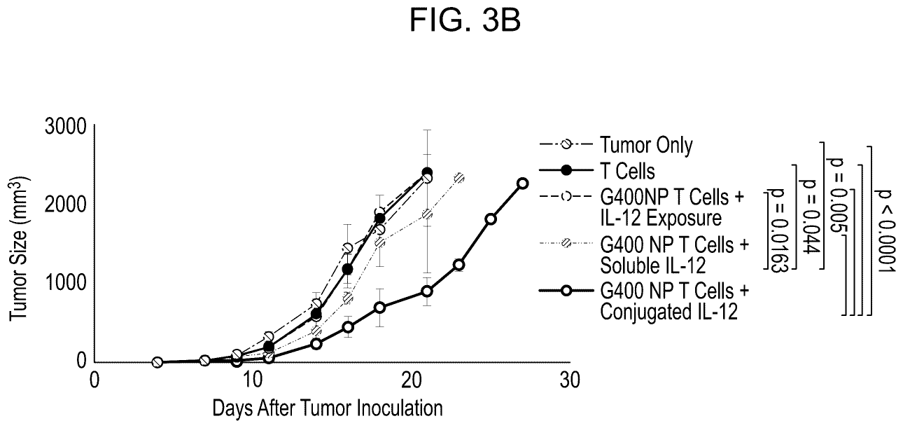

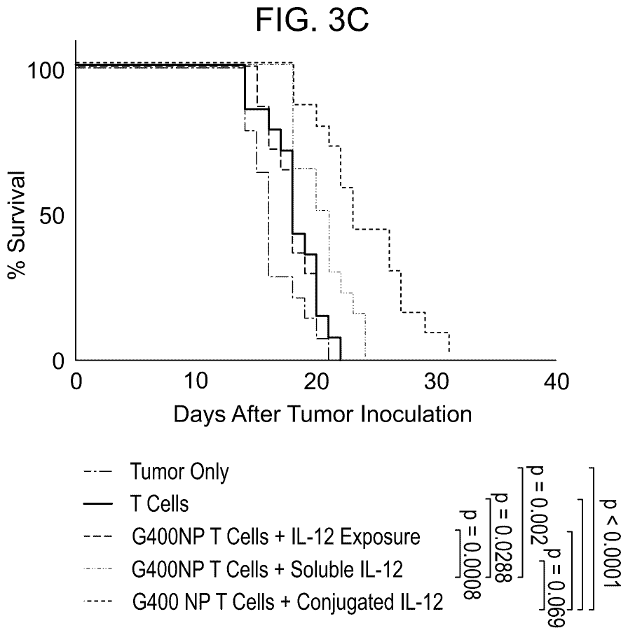

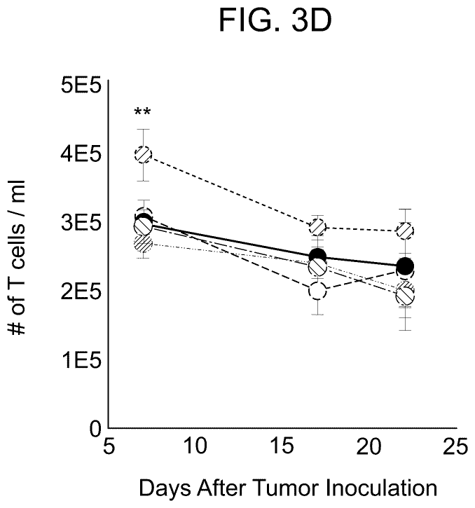

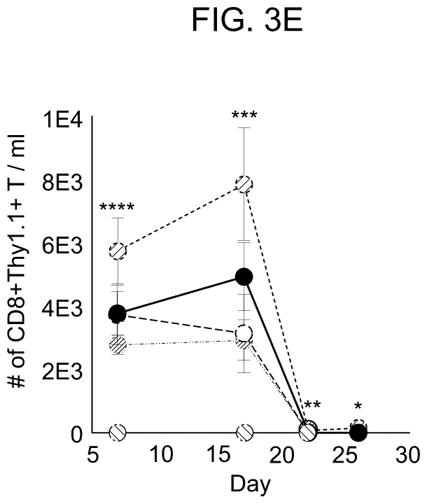

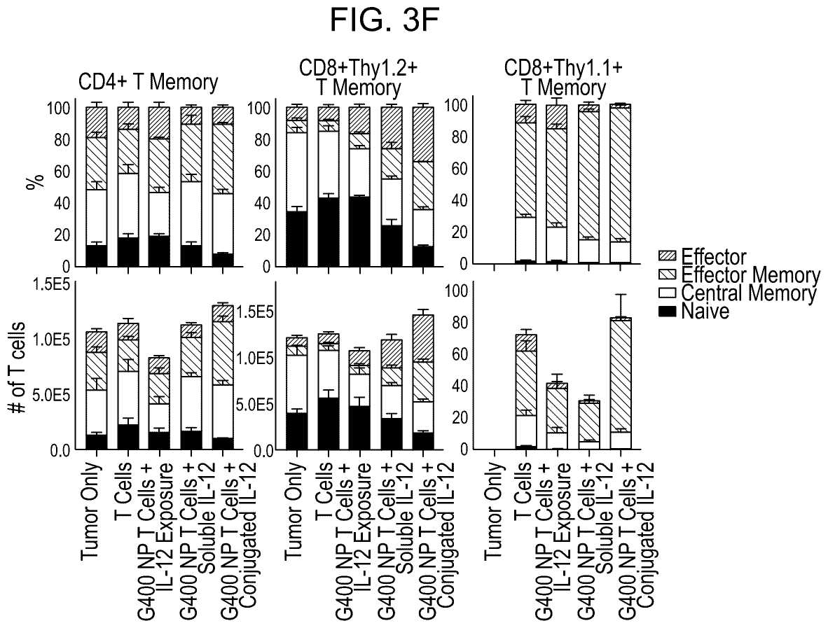

This experiment next tested whether conjugating anti-tumor cytokines onto T cells could provide benefits in controlling tumor growth in vivo, by adoptively transferring Pmel-1 TCR-transgenic gp100-specific T cells loaded with IL-12 in B16-F10 melanoma models. B16-F10 melanoma tumors were inoculated in the left flank of C57B1/6 (Thy1.2+) mice for 5 days before T cells were adoptively transferred intravenously (FIG. 3A). Injected T cells either were not modified at all (“T cells”), were treated with G400 NPs (“G400P”) and were exposed to unmodified cytokine for the reaction time (“G400P+Exposed IL-12”), were treated with G400 NPs and conjugated with DBCO-IL-12 (“G400P+Conjugated IL-12”), or were treated with G400 NPs and were injected with the same amount of IL-12 systemically (“G400P+Systematic IL-12”). Systemic injection of IL-12 at this dose offered minimal benefit in controlling tumor growth and prolonging mouse survival. However, conjugating IL-12 onto T cells significantly delayed tumor growth, and prolonged mice's life span by ˜50% (FIGS. 3B-3C). IL-12 conjugation onto T cells significantly increased the total number of T cells as well as gp-100 specific T cells at peak response (FIGS. 3D-3E). A closer look into the phenotypes of the T cells showed that mice receiving IL-12 conjugated T cells have a higher population of effector-like and effector-memory-like T cell both in terms of percentage and absolute count, and for both endogenous and adoptively transferred T cells (FIG. 3F).

### IL-12-Conjugated T Cells Promote Th1 Response, Antigen Presentation, and Antigen Spreading in the Endogenous Immune System

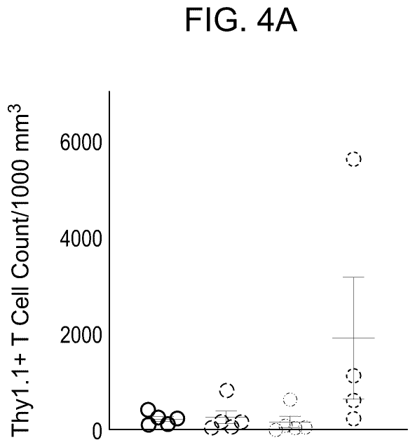

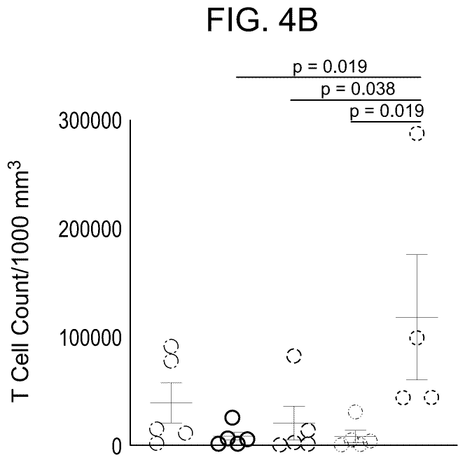

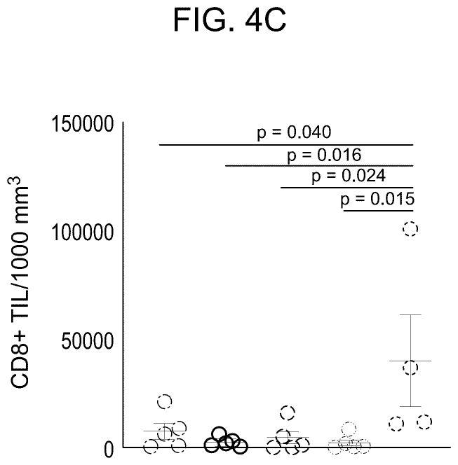

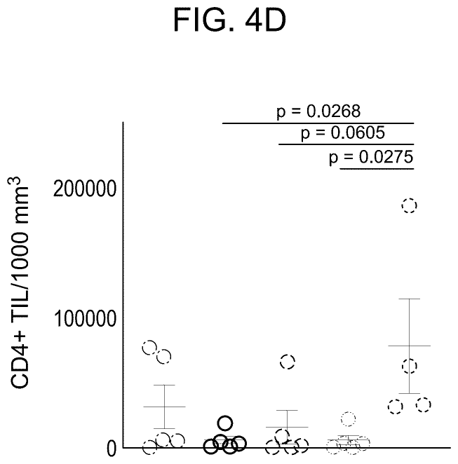

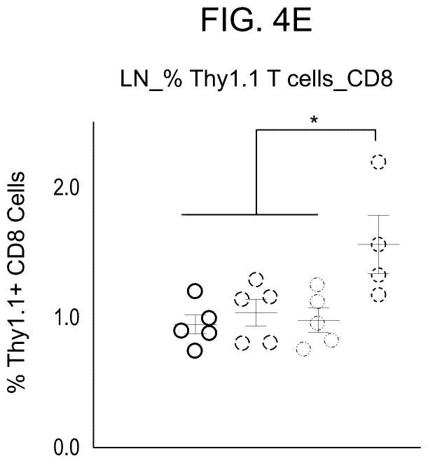

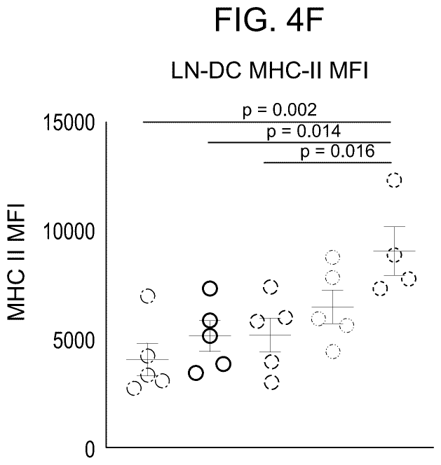

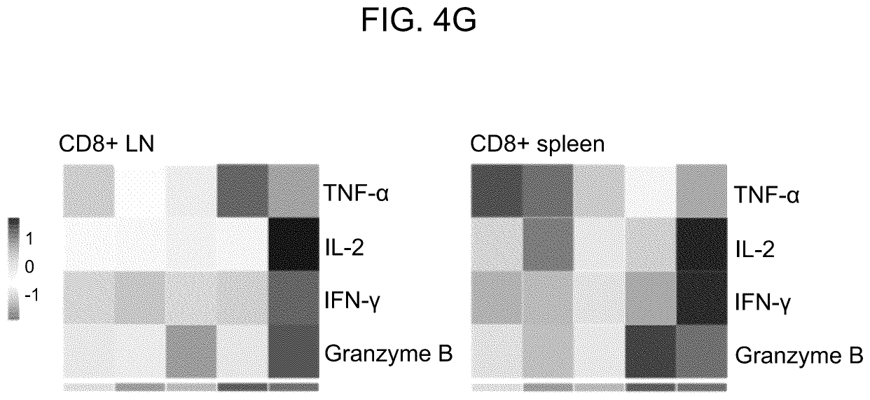

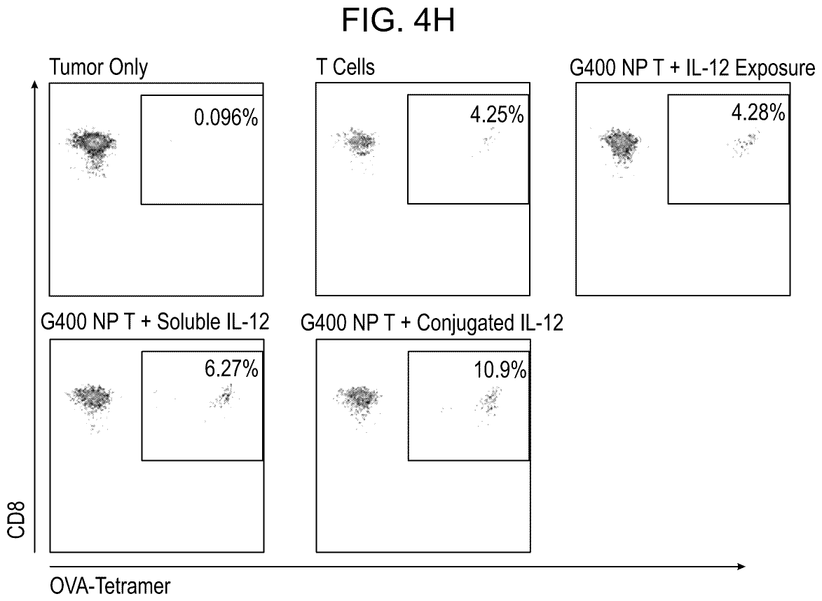

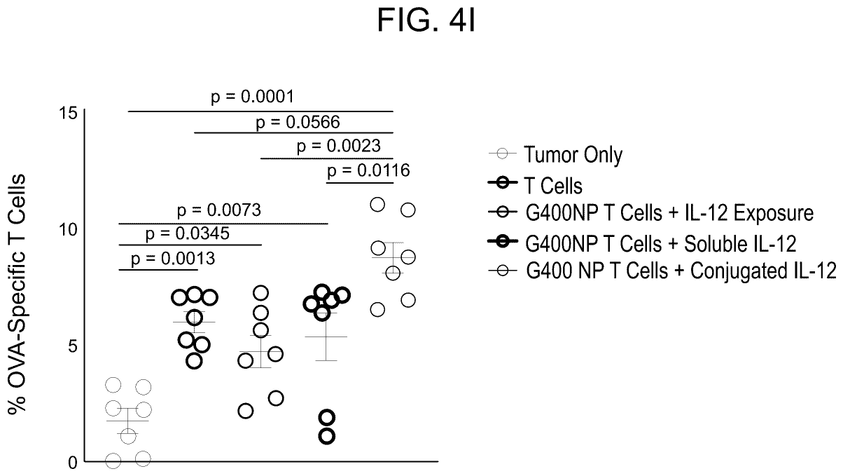

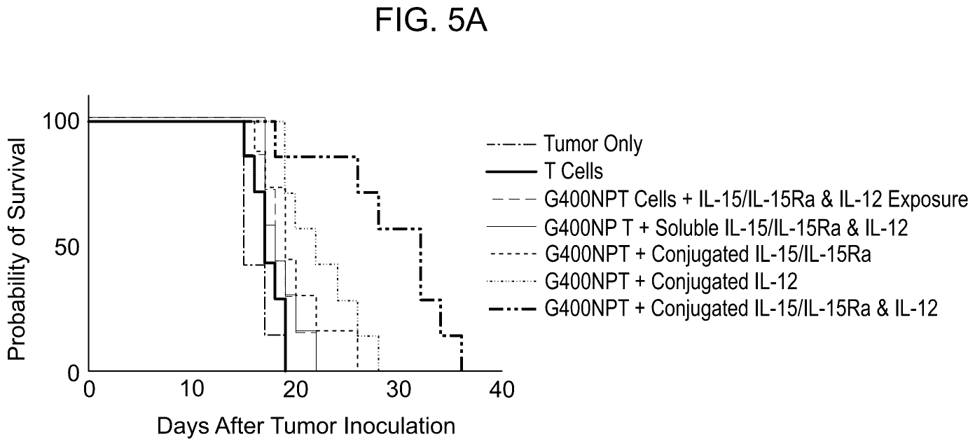

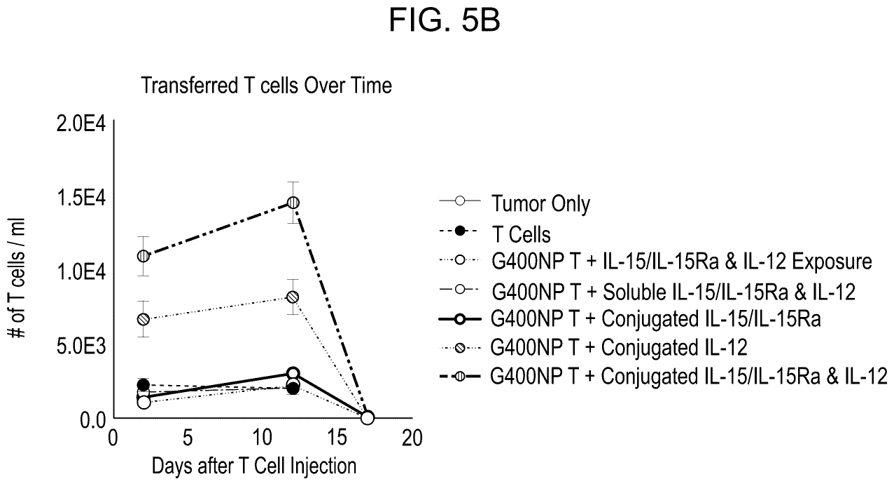

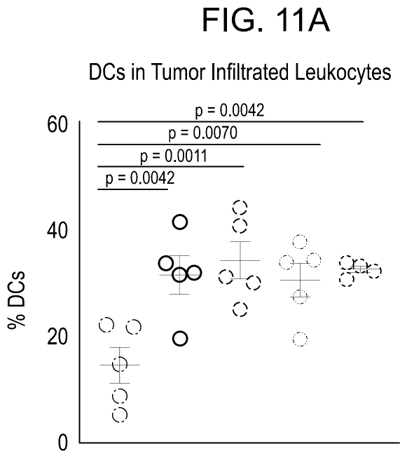

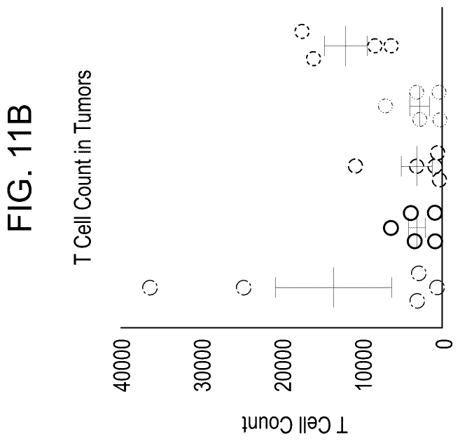

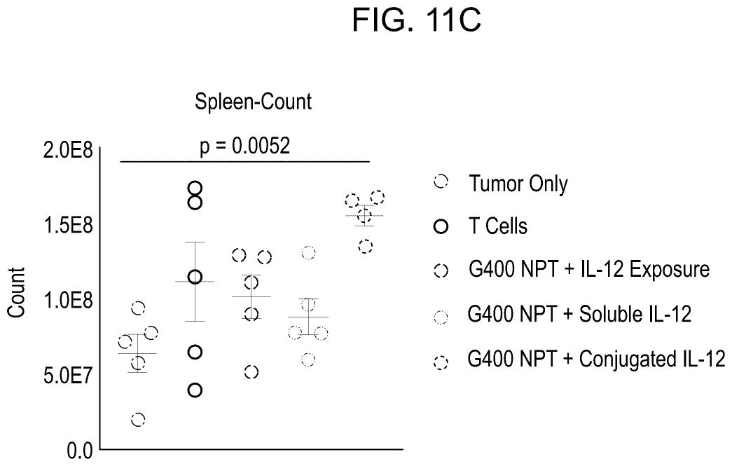

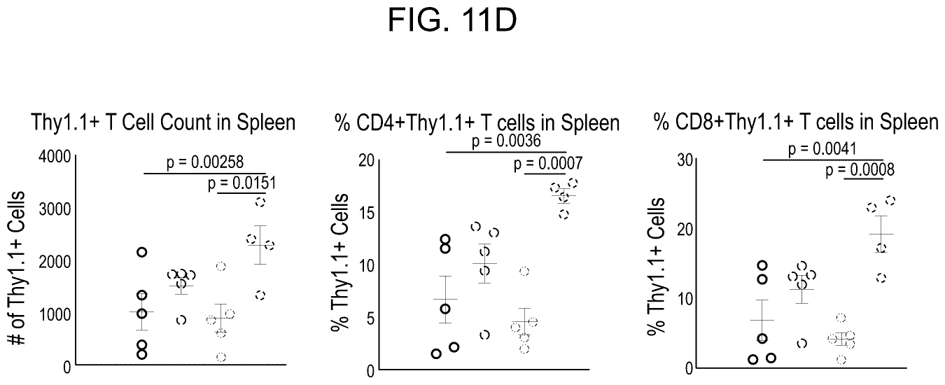

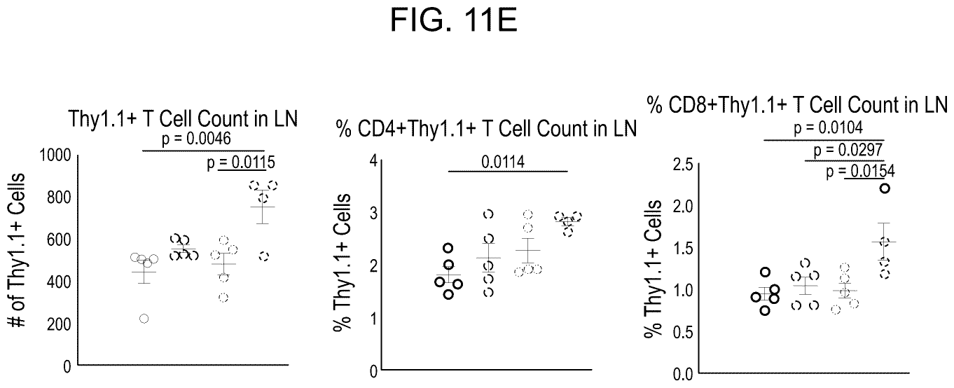

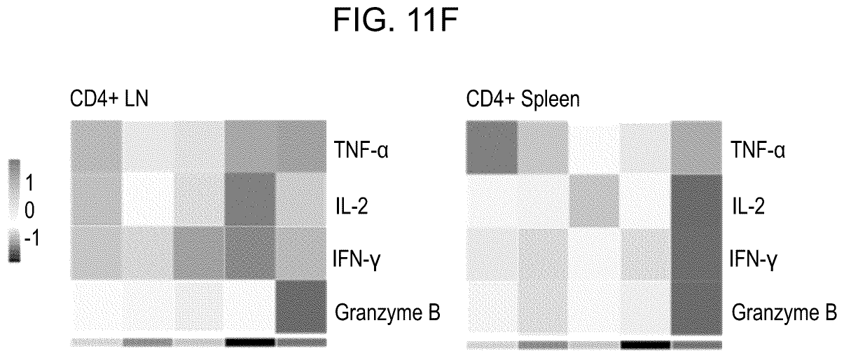

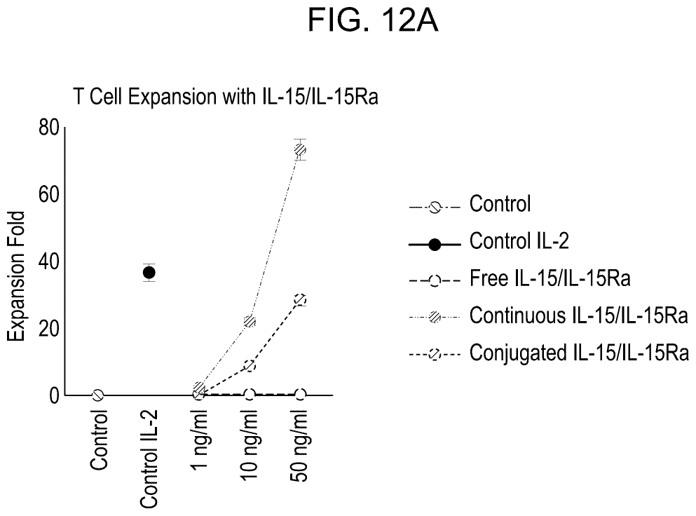

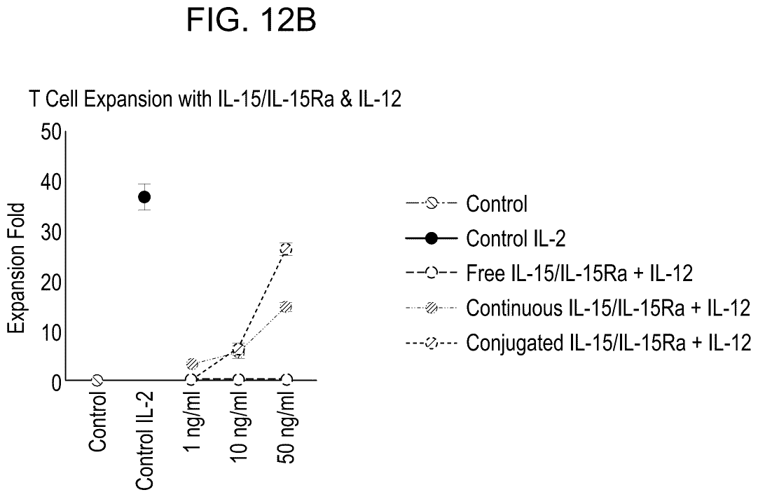

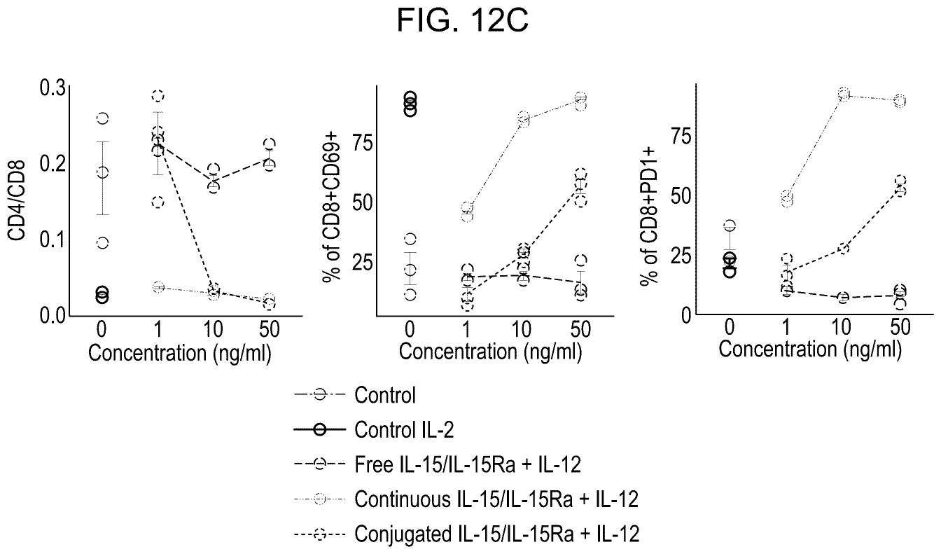

One interesting observation is that the difference in total T cell number could only be partially explained by the increase in Pmel-1 T cells, suggesting that the endogenous T cell population also benefited from adoptively transferred, IL-12 conjugated T cells (FIG. 3D). This prompted us to ask whether these adoptively transferred, IL-12 conjugated T cells play a role in activating the endogenous immune system as well. To answer this question, mice were inoculated with B16-F10 melanoma tumors for 5 days before T cells were adoptively transferred. On day 9 after T cells were transferred, mice were sacrificed, and tumors, spleens, and tumor-draining lymph nodes were collected and immune cells within were profiled. Within tumors, mice receiving adoptively transferred T cells have significantly more dendritic cells (DCs), but showed no difference between different conditions (FIG. 11A). However, we do observe a striking increase in number of adoptively transferred, Thy1.1+ T cells infiltrating the tumor when they received IL-12 conjugation, both in absolute T cell count and when T cell count was normalized to tumor volume (FIG. 4A, FIG. 11B). We also saw an increase in both CD4+ and CD8+ tumor infiltrating lymphocytes in tumors from mice that received T cells with IL-12 conjugation (FIGS. 4C-4D). Additionally, we saw larger spleens (FIG. 11C) as well as an increased percentage of Thy1.1+ T cells in lymph nodes and spleens when they were conjugated with IL-12 (FIGS. 11D-11E).

To better understand the performance of T cells isolated from spleen and tdLN in the context of tumor antigens, isolated T cells were co-cultured with B16-F10 melanoma cells for 4 hours, and stained for intracellular cytokines.

Interestingly, dendritic cells from tumor draining lymph nodes also expressed higher levels of MHC-II in mice that received IL-12 conjugated T cells, indicating that IL-12 conjugation helped with antigen presentation in the lymph nodes (FIG. 4F).

### Methods

### Animal Handling

Experiments and handling of mice were conducted under federal, state, and local guidelines and with approval from Harvard University's IACUC. Five- to six-week-old female C57B1/6 mice, TCR-transgenic Thy1.1+Pmel mice, and Nod/SCID/IL2RG^{-/-} (NSG) mice were purchased from the Jackson Laboratory.

### Preparation of G400 NP

G400 polymer was dissolved in dimethylformamide at 40 mg/ml, and added dropwise to ultrapure water on vigorous stirring. After 2 hours, G400 NP solution was dialyzed against deionized water for 48 h, concentrated with Amicon 3k centrifugal filters, and stored at 4° C. for future use.

### T Cell Isolation, Activation, Culturing, and Azido-Labeling

Mouse spleens were collected and kept on ice until and throughout processing. To obtain splenocytes, mouse spleen was mashed and passed through a 70 m strainer, washed with PBS, and centrifuged at 300 g for 5 min at 4° C. The pellet was resuspended in 1 ml ACK buffer (Lonza, #10-548E) for 1 min; 9 ml of PBS was then added and splenocytes collected by centrifuging at 300 g for 5 min at 4° C. Mouse Pan T cells and CD8+ T cells were isolated with magnetic-bead-based Pan T cell (Miltenyi #130-095-130) and CD8+ T cell (Miltenyi #130-104-075) isolation kit. Human T cell were isolated from PBMCs with magnetic-bead-based Pan T cell (Miltenyi #130-096-535) CD8+ T cell isolation kit (Miltenyi #130-096-495) following manufacturer protocol. Mouse and human CD8+ T cells were activated with Dynabeads mouse/human T-activator respectively (ThermoFisher Scientific #11452D/#11131D) and cultured in T cell media (RPMI 1640 (Lonza #BE12-702F), 10% heat-inactivate fetal bovine serum (Gibco #10-082-147), 1% pen/strep, 55 μM β-mercaptoethanol, 10 mM HEPES, 1% 100× non-essential amino acid (Lonza #13-144E), 100 mM sodium pyruvate (Lonza #13-115E), supplemented by mouse recombinant IL-2 (BioLegend #575406) or human recombinant IL-2 (BioLegend #589106) respectively (30-200 IU/ml). T cells were azido-labelled by adding G400 NP solution directly to T cell cultures at various concentrations for 72 hours.

### Flow Cytometry Analysis of Azido-Labeled T Cells

T cells that have been treated with G400 NP for 3 days were washed 3 times with PBS. For flow cytometry, T cells were stained for live/dead cells, washed with PBS, fixed, and stained with DBCO-AF594 at a concentration of 1 mM at 4° C. for 30 minutes. Stained T cells were then analyzed with flow.

Imaging and Azido-Signaling Analysis of Azido-Labeled T Cells with Confocal Imaging

T cells were treated with G400 NP for 3 days, after which T cells were washed 3 times with PBS, and kept in G400 NP-free media for culture. At each timepoint, T cells were taken, washed with PBS for 3 times, fixed and stained with DBCO-AF594 at a concentration of 1 mM at 4° C. for 30 minutes, and loaded onto coverslips with ProLong Gold Antifade mountant with DAPI for confocal imaging. Z-stacks of T cells were analyzed with IMARIS imaging analysis software. Cells were identified and added as elements for analysis based on DAPI (nuclei) and AF594 (cytosol) staining. The level of azido-labeling in each cell was determined by the median intensity of AF594 on the cell surface.

### DBCO-Modification of Cytokines and Verification

DBCO-IL-12, IL-21, TNF-α, and IL-15/IL-15Ra were obtained via reacting carrier-free cytokines with DBCO-sulfo-NHS at 1:8 molar ratio in PBS for 2 days at 4° C. The reaction mixture was then washed and concentrated with Amicon 3k centrifugal filters, and stored at −80° C. for future use.

Conjugating DBCO-Modified Cytokines onto T Cells

T cells were treated with 200 μM G400 NP for 3 days, and washed with PBS for 3 times. DBCO-cytokines were added to T cells for 30 minutes at 4° C. T cells were then washed for 3 times with PBS before downstream procedures.

### Flow Cytometry for In Vitro and In Vivo Cell Analysis

On first day of culture, CFSE staining was performed by adding T cells to 5 μM CFSE in PBS for 30 minutes at 37° C. for 20 minutes. Cells were then washed and kept in culture. Cells were stained with dead cell stain (ThermoFisher Scientific #L23105) according to manufacturer's protocol. Cells were then blocked with FcX Fc receptor blocking solution (BioLegend #101319, #422301) for 5 min and stained with surface protein antibodies for 20 min. Brilliant violet staining buffer (BD Horizon #563794) and flow cytometry staining buffer (Invitrogen #00-4222-26) were used during staining. Flow cytometry was then performed on BD Fortessa LSRII. Gating was performed based on fluorescence-minus-one controls. For blood samples from mice, 100 μl of ACK lysis buffer was added to every 50 μl of blood for 2 minutes to remove red blood cells. The cells were then washed and used for downstream staining.

### Cytolysis Assay

1 million mouse B16-F10 cells were stained with Calcein AM (Invitrogen #C₁₄₃₀) for 30 min at 37° C. in dark. B16-F10 melanoma cells were then washed 4 times with PBS, and kept in dark on ice until use. T cells from Pmel-1 mouse spleen were isolated, treated with G400 NP for 3 days and conjugated with various DBCO-cytokines. T cells and B16-F10 melanoma cells were seeded and well mixed in 96-well U-bottom plated at different E:T ratios in 200 μl media (RPMI 1640+10% FBS+1% P/S). The plate was centrifuged to collect cells to the bottom, and incubated at 37° C. for 6 hr. Supernatants were collected and their fluorescent signals measured with 485 nm excitation and 528 nm emission with a BioTek Synergy H1 plate reader.

### Measuring Cytokine Loading on T Cells

T cells were treated with G400 NP for 3 days. 300k T cells were seeded in a 96 well plate, and media containing various concentrations of DBCO-cytokine were added. T cells were placed at 4° C. for 30 min for click reaction to happen. Media before and after the reaction were collected, and ELISA was performed according to manufacturer's protocol to calculate the amount conjugated onto T cells.

### CAR-T Generation

Lentivirus construction and production: The 2nd generation CD19 CAR construct was composed of the scFv fragment from the FMC63 antibody (GenBank: ADM64594.1) fused to the human CD8a hinge and transmembrane region (Gene bank number NP_001759.3, aa 138-206) and linked to human 4-1BB (Gene bank number NP_001552.2, aa 214-255) and human CD3ζ (Gene bank number NP_000725, aa 52-163) intracellular signaling domains. To enable detection by flow cytometry, a cleavable truncated EGFR (tEGFR) was inserted to the N-terminus of the CD3ζ. Lentiviral supernatants were produced using the HEK 293T packaging line as previously described 32. 100% confluent HEK 293T cells in a well of a 6-well plate was co-transfected with 0.2 ug CAR-vector plasmid, 0.9 ug pMD2.G, 1.9 ug psPAX2 using lipofectamine 2000 (Life Technologies). The cultures were grown for 55 hr, after which the supernatants were collected, filtered to remove debris, and frozen at −80 C before use.

CAR-T transduction: T cells were isolated from healthy donors using the human pan-T cell isolation kit (Miltenyi Biotec) to obtain CD3+ T cells. Isolated T cells were activated with Dynabead (ThermoFisher Scientific 111161D) at 1:1 ratio. After 48 h, activated T cells were transduced by adding 140 μL of pre-warmed lentiviral supernatant containing the CD19 CAR construct. After 36 h, the media containing T cells and any remaining material were transferred to a 6-well G-Rex plate (Wilson Wolf) containing pre-warmed T cell media (described above) and expanded for 3 days. Transduced T cells were magnetically separated from Dynabeads and cryopreserved in 10% DMSO+90% FBS. The CAR-T transduction efficiency is consistently around 25%.

### B16-F10 Melanoma Model

Tumor inoculation and Pmel T cell treatment: 100k B16-F10 melanoma cells are injected subcutaneously on the left flank of the C57B1/6 mice (female, −6 weeks of age, Jacksons Laboratory). After 5 days, tumor bearing mice were randomized and injected with either PBS, or 7 million Thy1.1+Pmel T cells treated under different conditions via tail vein injection.

Tumor monitoring: size of B16-F10 melanoma was monitored over time by measuring the height, width, and length of the tumor. The total size of tumor is calculated as height*width*length*0.5. Mice were euthanized if the total tumor size over 2,000 mm³, if any of the tumor dimensions over 20 mm, or if significant discomfort or weight loss was observed.

Tracking of adoptively transferred T cells: Each week, animals were bled via the tail vein and ˜50 μL blood was collected in K2-EDTA-coated collection tubes (BD). The samples were treated with ACK lysis buffer (Lonza, BioLegend), washed, and processed for flow cytometry as described above.

### Xenograft Lymphoma Model

Tumor inoculation and CAR-T cell treatment: Female NSG mice, between 6-7 wks of age were inoculated with a high dose of 5×105 luciferized Raji cells (Raji-luc) intravenously on day 0. After 4 days, tumor-bearing mice were randomized into treatment groups and were treated with either mock (PBS) or 5×105 CAR+ T cells treated under different conditions Tumor tracking: Raji-luc tumor burden was monitored over time using D-Luciferin (Gold Biotechnology). Animals were anesthetized and intraperitoneally injected with D-Luciferin at 150 mg/kg. Luminescence was measured 10 minutes post injection via IVIS (Perkin Elmer). Total flux (p/s) per mouse was quantified in mouse whole-body regions-of-interest (ROI). Animals were imaged once every 4 to 14 days and their weights were simultaneously quantified. Mice were euthanized if flux from tumor is larger than 1E11, or if significant discomfort or weight loss was observed.

Tracking of adoptively transferred CAR-T cells. Each week, animals were bled via the tail vein and ˜50 μL blood was collected in K2-EDTA-coated collection tubes (BD). The samples were treated with ACK lysis buffer (Lonza, BioLegend), washed, and processed for flow cytometry as described above.

Cell Isolation from Tumor and Lymph Node

Tumor and tumor-draining lymph node was separated from mouse and mashed into small pieces. Mashed tissues were treated with 200 IU collagenase type I at 37° C. for 1 hour, and were passed through pipette tips every 20 minutes. After collagenase type IV treatment, the solutions were passed through a 70 μM filter to achieve single cell suspension. Cells from lymph node were then used for downstream analysis. Cells from tumor were spun down and treated with ACK lysis buffer to remove red blood cells, and resuspended in 1 mL RPMI media. This single cell suspension was gently added to the top of 2 mL 40% (v/v) Percoll and 2 mL 70% (v/v) Percoll to create a separation, and centrifuged at 800 g for 30 minutes at room temperature. Tumor infiltrating lymphocytes were collected from the middle layer and washed with ice-cold PBS for three times before they were used for downstream analysis.

### T Cell Activation Assay and Intracellular Cytokine Staining

Mouse T cells isolated from tumor, spleen, and lymph nodes were co-cultured with B16-F10 melanoma T cells in 100 μl media. The plate is centrifuged at 1600 rpm to collect cells to the bottom. After 1 hour, GolgiPlug (BD #555029) was added according to manufacturer's protocol to stop cytokine secretion. 3 hours later, T cell and tumor cell mixtures were washed, and stained with dead cell stain, surface proteins, and intracellular cytokines with Cyto-Fast fix-perm buffer set (BioLegend #426803) according to manufacturer's protocol.

### Tetramer Staining

Blood samples from mice were treated with ACK lysis buffer to remove red blood cells. 10 μl of SIINFEKL tetramer (TB-5001-4) were added to each sample, the final volume was adjusted to 200 μl with PBS, and the sample was incubated for 20 min at 37° C. Primary antibodies for other cell surface markers were then added and incubated for 20 min at 4° C. Cells were then washed with PBS for three times and stained with live/dead staining, and used for flow cytometry analysis.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the present invention described herein. Such equivalents are intended to be encompassed by the following claims.

1. A method of preventing or treating a cancer in a subject, of enhancing an immune response against a cancer in a subject, or of delivering a non-toxic level of cytokine to a subject, comprising administering to the subject an adoptive cell therapy, wherein the adoptive cell therapy comprises:
(i) an immune cell comprising a cell-surface glycoprotein coupled to a first click reagent; and
(ii) a cytokine coupled to a second click reagent,
wherein the cell-surface glycoprotein is covalently linked to the cytokine through a selective reaction between the first click reagent and the second click reagent,
thereby preventing or treating the cancer, enhancing an immune response against the cancer or delivering the non-toxic level of cytokine to the subject.

2. (canceled)

3. (canceled)

4. The method of claim 1, further comprising contacting the immune cell with an unnatural sugar and/or an unnatural sugar nanoparticle to produce the immune cell comprising the cell-surface glycoprotein coupled to a first click reagent.

5. A method of treating a subject in need thereof with an adoptive cell therapy, comprising:
(i) contacting an immune cell with an unnatural sugar and/or an unnatural sugar nanoparticle to produce an immune cell comprising a cell-surface glycoprotein coupled to a first click reagent; and
(ii) administering to the subject the immune cell comprising the cell-surface glycoprotein coupled to the first click reagent, and a cytokine coupled to a second click reagent,
wherein the cell-surface glycoprotein is covalently linked to the cytokine through a selective reaction between the first click reagent and the second click reagent,
thereby treating a subject in need thereof with an adoptive cell therapy.

6. The method of claim 1,
(i) wherein the first click reagent is selected from the group consisting of an azide group, a dibenzocyclooctyne (DBCO) group, a transcyclooctene group, a tetrazine group, a norbornene group, and variants thereof, optionally wherein the first click reagent comprises an azide group;
(ii) wherein the second click reagent is selected from the group consisting of an azide group, a dibenzocyclooctyne (DBCO) group, a transcyclooctene group, a tetrazine group, a norbornene group, and variants thereof;
(iii) wherein the second click reagent comprises a dibenzocyclooctyne (DBCO) group; and/or
(iv) wherein the first click reagent comprises an azide group and the second click reagent comprises a dibenzocyclooctyne (DBCO) group.

7. The method of claim 4, wherein the unnatural sugar and/or an unnatural sugar nanoparticle comprises the first click reagent, optionally wherein the unnatural sugar and/or an unnatural sugar nanoparticle is an unnatural azido-sugar and/or an unnatural azido-sugar nanoparticle.

8.-10. (canceled)

11. The method of claim 6, (i) wherein the DBCO group is coupled to a primary amine of the cytokine; and/or (ii) wherein the cytokine is coupled to between 1-10 DBCO groups, optionally wherein the cytokine is coupled to 1, 2, or 3 DBCO groups.

12. (canceled)

13. The method of claim 1, wherein:
(i) the cytokine is selected from the group consisting of interleukins, interferons, chemokines, tumor necrosis factors, and colony stimulating factors of immune cell precursors;
(ii) the cytokine is an interleukin selected from the group consisting of IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, and IL-35;
(iii) the cytokine is a chemokine selected from the group consisting of CCL family, CXCL family, CX3CL family, and XCL family chemokines.
(iv) the cytokine is an inflammatory cytokine selected from the group consisting of IFN-γ, IL-1, and TNF-α;
(v) the cytokine is selected from the group consisting of interleukin-2 (IL-2), interleukin-7 (IL-7), interleukin-10 (IL-10), interleukin-12 (IL-12), interleukin-15 (IL-15), IL-15/IL-15Rα, interleukin-18 (IL-18), interleukin-21 (IL-21), interleukin-27 (IL-27), interferon-α (IFN-α), interferon- [symbol] (IFN- [symbol] ), granulocyte macrophage-colony stimulating factor (GM-CSF), Fms-like tyrosine kinase receptor 3 ligand (Flt3-L), tumour necrosis factor α (TNF-α), and combinations thereof;
(vi) the cytokine is an anti-tumor cytokine;
(vii) the cytokine is IL-2; and/or
(viii) the cytokine is administered to the subject prior to, concurrently with, or after the administration of the immune cell.

14. (canceled)

15. (canceled)

16. The method of claim 1, wherein the selective reaction between the first click reagent and the second click reagent occurs in vitro, ex vivo, or in vivo.

17. The method of claim 1,
(i) wherein the immune cell comprises a lymphocyte, optionally a tumor-infiltrating lymphocyte;
(ii) wherein the immune cell comprises a T-cell, a B-cell, a natural killer (NK) cell, a regulatory T (Treg) cell, or a combination thereof; or
(iii) wherein the immune cell comprises an engineered T cell receptor (TCR) and/or a chimeric antigen receptor (CAR).

18. (canceled)

19. (canceled)

20. The method of claim 1, wherein the adoptive cell therapy is selected from the group consisting of
(i) a tumor-infiltrating lymphocyte (TIL) therapy;
(ii) a engineered T cell receptor (TCR) therapy;
(iii) a chimeric antigen receptor (CAR) T cell therapy;
(iv) a natural killer (NK) cell therapy; and
(v) a regulatory T (Treg) cell therapy.

21. The method of claim 1, which reduces tumor size, delays tumor growth, reduces cancer burden, increases survival time, prevents cancer from developing, depletes cancer cells, prevents or reduces cancer relapse, or prevents or reduces cancer recurrence or metastasis, increases T cells infiltration in solid tumors, increases antigen presentation, and/or increases antigen spreading in the subject; and/or
which results in the targeted delivery of non-toxic levels of one or more cytokines to the subject and/or which reduces cytokine-related toxicity and inhibition on immune cell proliferation as compared with systemic cytokine administration.

22. (canceled)

23. The method of claim 1, wherein the subject is suffering from a cancer, a viral disease, and/or an autoimmune disease.

24. The method of claim 23, wherein the cancer is selected from the group consisting of a cancer of the digestive system; a hepatic carcinoma; a liver cancer; a colon cancer; an esophageal cancer; a gastric cancer; a hepatoma; a kidney or renal cancer; an oral cavity cancer; a pancreatic cancer; a prostate cancer; a rectal cancer; a stomach cancer; a basal cell carcinoma; a biliary tract cancer; a lung cancer; a bladder cancer; a cervical cancer; an endometrial cancer; a uterine cancer; a blond cancer; a bone cancer; a skin cancer; a cancer of the urinary system; and combinations thereof, optionally wherein the cancer is selected from the group consisting of a solid tumor, a leukemia, a lymphoma, and a multiple myeloma, optionally wherein the cancer comprises a solid tumor.

25. The method of claim 1, wherein the immune cell is administered to the subject prior to, concurrently with, or after the administration of a scaffold, optionally wherein the scaffold comprises an additional agent selected from the group consisting of a growth factor, a differentiation factor, a homing factor, a cytokine, a chemokine, and combinations thereof.

26. The method of claim 25, wherein the selective reaction between the first click reagent and the second click reagent occurs in vitro, ex vivo, or in vivo within a scaffold.

27. A method of producing an adoptive cell therapy, comprising:
(i) providing an immune cell comprising a cell-surface glycoprotein;
(ii) contacting the immune cell with an unnatural azido-sugar and/or an unnatural azido-sugar nanoparticle to metabolically label the cell-surface glycoprotein with a first click reagent; and
(iii) contacting the immune cell with a cytokine coupled to a second click reagent,
wherein the cell-surface glycoprotein is covalently linked to the cytokine through a selective reaction between the first click reagent and the second click reagent,
thereby producing the adoptive cell therapy.

28. The method of claim 27, wherein the unnatural azido-sugar nanoparticle comprises a polymer of azido sugar;
optionally, wherein the polymer of azido sugar comprises a tetraacetyl-N-azidoacetylmannosamine (Ac₄ManAz) or a derivative thereof;
optionally, wherein the Ac₄ManAz is functionalized with at least one acrylate bond;
optionally, wherein the polymer of azido sugar is produced by reversible addition-fragmentation chain-transfer (RAFT) polymerization of Ac₄ManAz to yield poly(azido-sugar)ₙ, wherein n is any integer between 1 and 500 (n=1 (G1) or n=500 (G500));
optionally, wherein the unnatural azido-sugar nanoparticle comprises a G400 nanoparticle.

29-32. (canceled)

33. An immune cell comprising:
(i) a cell-surface glycoprotein coupled to a first click reagent; and
(ii) a cytokine coupled to a second click reagent;
wherein the cell-surface glycoprotein is covalently linked to the cytokine through a selective reaction between the first click reagent and the second click reagent.

34. The cell of claim 33,
(i) wherein the immune cell comprises a lymphocyte, optionally a tumor-infiltrating lymphocyte;
(ii) wherein the immune cell comprises a T-cell, a B-cell, a natural killer (NK) cell, a regulatory T (Treg) cell, or a combination thereof; or
(iii) wherein the immune cell comprises an engineered T cell receptor (TCR) and/or a chimeric antigen receptor (CAR).

35. (canceled)

36. (canceled)

37. A composition comprising the immune cell of claim 33.
